(19) United States
(12) Patent Application Publication
(10) Pub. No.: US 2014/0295513 A1
(43) Pub. Date: Oct. 2, 2014

(54) High-Performance Ketol-Acid Reductoisomerases

(71) Applicants: Peter Meinhold, Topanga, CA (US); Doug Lies, Englewood, CO (US); Stephanie Porter-Scheinman, Conifer, CO (US); Christopher Smith, Parker, CO (US); Christopher Snow, Englewood, CO (US); Sabine Bastian, Pasadena, CA (US); Sebastian Schoof, Pasadena, CA (US)

(72) Inventors: Peter Meinhold, Topanga, CA (US); Doug Lies, Englewood, CO (US); Stephanie Porter-Scheinman, Conifer, CO (US); Christopher Smith, Parker, CO (US); Christopher Snow, Englewood, CO (US); Sabine Bastian, Pasadena, CA (US); Sebastian Schoof, Pasadena, CA (US)

(73) Assignees: CALIFORNIA INSTITUTE OF TECHNOLOGY, Pasadena, CA (US); GEVO, INC., Englewood, CO (US)

(21) Appl. No.: 14/131,984

(22) PCT Filed: Jul. 11, 2012
(86) PCT No.: PCT/US12/46185

§ 371 Date: Jun. 19, 2014

Related U.S. Application Data
(63) Continuation of application No. 13/303,884, filed on Nov. 23, 2011.
(60) Provisional application No. 61/506,562, filed on Jul. 11, 2011.
(60) Provisional application No. 61/506,564, filed on Jul. 11, 2011.
(60) Provisional application No. 61/510,618, filed on Jul. 22, 2011.

(51) Int. Cl. C12P 7/16; C12N 9/04

(52) U.S. Cl. CPC C12P 7/16; C12N 9/0006
USPC 435/160; 435/190; 435/254.2; 536/23.1

(57) ABSTRACT

The present invention relates to recombinant microorganisms comprising at least one nucleic acid molecule encoding a ketol-acid reductoisomerase (KARI) or a modified NADH-dependent variant thereof, wherein said KARI is at least about 80% identical to SEQ ID NO: 2, SEQ ID NO: 4, SEQ ID NO: 6, SEQ ID NO: 8, SEQ ID NO: 10, SEQ ID NO: 28, SEQ ID NO: 40, or SEQ ID NO: 58. The present invention also relates to recombinant microorganisms comprising at least one nucleic acid molecule encoding a ketol-acid reductoisomerase (KARI) or a modified NADH-dependent variant thereof, wherein said KARI is at least about 99% identical to SEQ ID NO: 64. In various aspects of the invention, the recombinant microorganisms may comprise an isobutanol producing metabolic pathway and can be used in methods of making isobutanol.

### CROSS-REFERENCE TO RELATED APPLICATIONS

This application claims priority to U.S. Provisional Application Ser. No. 61/506,562, filed Jul. 11, 2011, U.S. Provisional Application Ser. No. 61/506,564, filed Jul. 11, 2011, U.S. Provisional Application Ser. No. 61/510,618, filed Jul. 22, 2011, and is a continuation-in-part of U.S. Non-Provisional application Ser. No. 13/303,884, filed Nov. 23, 2011, each of which is herein incorporated by reference in its entirety for all purposes.

### ACKNOWLEDGMENT OF GOVERNMENTAL SUPPORT

This invention was made with government support under Contract No. 2009-10006-05919, awarded by the United States Department of Agriculture, and under Contract No. W911NF-09-2-0022, awarded by the United States Army Research Laboratory. The government has certain rights in the invention.

### TECHNICAL FIELD

Recombinant microorganisms and methods of producing such microorganisms are provided. Also provided are methods of producing beneficial metabolites including fuels and chemicals by contacting a suitable substrate with the recombinant microorganisms of the invention and enzymatic preparations therefrom.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: GEVO_{—}064_{—}01WO_SeqList_ST25.txt, date recorded: Jul. 10, 2012, file size: 185 kilobytes).

### BACKGROUND

The ability of microorganisms to convert sugars to beneficial metabolites including fuels, chemicals, and amino acids has been widely described in the literature in recent years. See, e.g., Alper et al., 2009, Nature Microbiol. Rev. 7: 715-723 and McCourt et al., 2006, Amino Acids 31: 173-210. Recombinant engineering techniques have enabled the creation of microorganisms that express biosynthetic pathways capable of producing a number of useful products, including the commodity chemical, isobutanol.

Isobutanol, also a promising biofuel candidate, has been produced in recombinant microorganisms expressing a heterologous, five-step metabolic pathway (See, e.g., WO/2007/050671 to Donaldson et al., WO/2008/098227 to Liao et al., and WO/2009/103533 to Festel et al.). However, the microorganisms produced to date have fallen short of commercial relevance due to their low performance characteristics, including, for example low productivities, low titers, and low yields.

The second step of the isobutanol producing metabolic pathway is catalyzed by ketol-acid reductoisomerase (KARI), which converts acetolactate to 2,3-dihydroxyisovalerate. Because KARI is an essential enzyme in the isobutanol production pathway, it is desirable that recombinant microorganisms engineered to produce isobutanol exhibit optimal KARI activity. The present application addresses this need by identifying several KARI enzymes that give high performance with an isobutanol production pathway expressed in yeast. Accordingly, this application describes methods of increasing isobutanol production through the use of recombinant microorganisms comprising KARI enzymes with improved properties for the production of isobutanol.

Another important feature of a KARI enzyme is the ability to use NADH as a cofactor for the conversion of acetolactate to 2,3-dihydroxyisovalerate. The present inventors have found that when an NADH-dependent KARI is used in conjunction with an NADH-dependent alcohol dehydrogenase (ADH), isobutanol can be produced at theoretical yield and/or under anaerobic conditions. See, e.g., commonly owned and co-pending US Publication No. US 2010/0143997. Because NADH-dependence is an important feature of a KARI enzyme, the present inventors have identified several beneficial mutations which can be made to the KARI enzymes identified herein to switch the cofactor specificity of the enzymes from NADPH to NADH.

### SUMMARY OF THE INVENTION

The present inventors have discovered a group of KARI enzymes with high level activity in the isobutanol pathway. The use of one or more of these KARI enzymes, or NADH-dependent variants thereof, can improve production of the isobutanol.

In a first aspect, the application relates to a recombinant microorganism comprising at least one nucleic acid molecule encoding a ketol-acid reductoisomerase (KARI), wherein said KARI is at least about 80% identical to SEQ ID NO: 2. In one embodiment, the KARI is derived from the genus Shewanella. In a specific embodiment, the KARI is derived from Shewanella sp. strain MR-4. In another specific embodiment, the KARI is encoded by SEQ ID NO: 1.

In another aspect, the application relates to a recombinant microorganism comprising at least one nucleic acid molecule encoding a ketol-acid reductoisomerase (KARI), wherein said KARI is at least about 80% identical to SEQ ID NO: 4. In one embodiment, the KARI is derived from the genus Vibrio. In a specific embodiment, the KARI is derived from Vibrio fischeri. In another specific embodiment, the KARI is encoded by SEQ ID NO: 3.

In another aspect, the application relates to a recombinant microorganism comprising at least one nucleic acid molecule encoding a ketol-acid reductoisomerase (KARI), wherein said KARI is at least about 80% identical to SEQ ID NO: 6. In one embodiment, the KARI is derived from the genus Gramella. In a specific embodiment, the KARI is derived from Gramella forsetii. In another specific embodiment, the KARI is encoded by SEQ ID NO: 5.

In another aspect, the application relates to a recombinant microorganism comprising at least one nucleic acid molecule encoding a ketol-acid reductoisomerase (KARI), wherein said KARI is at least about 80% identical to SEQ ID NO: 8. In one embodiment, the KARI is derived from the genus Cytophaga. In a specific embodiment, the KARI is derived from Cytophaga hutchinsonii. In another specific embodiment, the KARI is encoded by SEQ ID NO: 7.

In another aspect, the application relates to a recombinant microorganism comprising at least one nucleic acid molecule encoding a ketol-acid reductoisomerase (KARI), wherein said KARI is at least about 80% identical to SEQ ID NO: 10. In one embodiment, the KARI is derived from a genus selected from Lactococcus and Streptococcus. In a specific embodiment, the KARI is derived from Lactococcus lactis, Streptococcus equinus, or Streptococcus infantarius. In another specific embodiment, the KARI is encoded by SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25.

In another aspect, the application relates to a recombinant microorganism comprising at least one nucleic acid molecule encoding a ketol-acid reductoisomerase (KARI), wherein said KARI is at least about 80% identical to SEQ ID NO: 28. In one embodiment, the KARI is derived from the genus Methanococcus. In a specific embodiment, the KARI is derived from Methanococcus maripaludis, Methanococcus vannielii, or Methanococcus voltae. In another specific embodiment, the KARI is encoded by SEQ ID NO: 27, SEQ ID NO: 29, SEQ ID NO: 31, SEQ ID NO: 33, SEQ ID NO: 35, or SEQ ID NO: 37.

In another aspect, the application relates to a recombinant microorganism comprising at least one nucleic acid molecule encoding a ketol-acid reductoisomerase (KARI), wherein said KARI is at least about 80% identical to SEQ ID NO: 40. In one embodiment, the KARI is derived from a genus selected from Zymomonas, Erythrobacter, Sphingomonas, Sphingobium, and Novosphingobium. In a specific embodiment, the KARI is derived from Zymomonas mobilis, Erythrobacter litoralis, Sphingomonas wittichii, Sphingobium japonicum, Sphingobium chlorophenolicum, or Novosphingobium nitrogenifigens. In another specific embodiment, the KARI is encoded by SEQ ID NO: 39, SEQ ID NO: 41, SEQ ID NO: 43, SEQ ID NO: 45, SEQ ID NO: 47, SEQ ID NO: 49, SEQ ID NO: 51, or SEQ ID NO: 53.

In another aspect, the application relates to a recombinant microorganism comprising at least one nucleic acid molecule encoding a ketol-acid reductoisomerase (KARI), wherein said KARI is at least about 80% identical to SEQ ID NO: 56. In one embodiment, the KARI is derived from the genus Bacteroides. In a specific embodiment, the KARI is derived from Bacteroides thetaiotaomicron. In another specific embodiment, the KARI is encoded by SEQ ID NO: 55.

In another aspect, the application relates to a recombinant microorganism comprising at least one nucleic acid molecule encoding a ketol-acid reductoisomerase (KARI), wherein said KARI is at least about 80% identical to SEQ ID NO: 58. In one embodiment, the KARI is derived from the genus Schizosaccharomyces. In a specific embodiment, the KARI is derived from Schizosaccharomyces pombe or Schizosaccharomyces japonicus. In another specific embodiment, the KARI is encoded by SEQ ID NO: 57, SEQ ID NO: 59, or SEQ ID NO: 61.

In another aspect, the application relates to a recombinant microorganism comprising at least one nucleic acid molecule encoding a ketol-acid reductoisomerase (KARI), wherein said KARI is at least about 99% identical to SEQ ID NO: 64. In one embodiment, the KARI is derived from the genus Salmonella. In a specific embodiment, the KARI is derived from Salmonella enterica. In another specific embodiment, the KARI is encoded by SEQ ID NO: 63.

In some embodiments, the KARI may be modified to be NADH-dependent. Accordingly, the present application further relates to NADH-dependent ketol-acid reductoisomerases (NKRs) derived from a KARI that is at least about 80% identical to SEQ ID NO: 2, SEQ ID NO: 4, SEQ ID NO: 6, SEQ ID NO: 8, SEQ ID NO: 10, SEQ ID NO: 28, SEQ ID NO: 40, or SEQ ID NO: 58. Thus, in one embodiment, the present application relates to a recombinant microorganism comprising a NKR derived from a KARI that is at least about 80% identical to SEQ ID NO: 2, SEQ ID NO: 4, SEQ ID NO: 6, SEQ ID NO: 8, SEQ ID NO: 10, SEQ ID NO: 28, SEQ ID NO: 40, or SEQ ID NO: 58.

In other embodiments, the present application further relates to NADH-dependent ketol-acid reductoisomerases (NKRs) derived from a KARI that is at least about 99% identical to SEQ ID NO: 64. Thus, in one embodiment, the present application relates to a recombinant microorganism comprising a NKR derived from a KARI that is at least about 99% identical to SEQ ID NO: 64.

Therefore, the present application also relates to mutated ketol-acid reductoisomerase (KARI) enzymes that utilize NADH rather than NADPH.

Examples of such KARIs include enzymes having one or more modifications or mutations at positions corresponding to amino acids selected from: (a) alanine 71 of the Shewanella sp. KARI (SEQ ID NO: 2); (b) arginine 76 of the Shewanella sp. KARI (SEQ ID NO: 2); (c) serine 78 of the Shewanella sp. KARI; and (d) glutamine 110 of the Shewanella sp. KARI (SEQ ID NO: 2).

In one embodiment, the KARI enzyme contains a modification or mutation at the amino acid corresponding to position 71 of the Shewanella sp. KARI (SEQ ID NO: 2). In another embodiment, the KARI enzyme contains a modification or mutation at the amino acid corresponding to position 76 of the Shewanella sp. KARI (SEQ ID NO: 2). In yet another embodiment, the KARI enzyme contains a modification or mutation at the amino acid corresponding to position 78 of the Shewanella sp. KARI (SEQ ID NO: 2). In yet another embodiment, the KARI enzyme contains a modification or mutation at the amino acid corresponding to position 110 of the Shewanella sp. KARI (SEQ ID NO: 2). In one embodiment, the KARI enzyme contains two or more modifications or mutations at the amino acids corresponding to the positions described above. In another embodiment, the KARI enzyme contains three or more modifications or mutations at the amino acids corresponding to the positions described above. In yet another embodiment, the KARI enzyme contains four modifications or mutations at the amino acids corresponding to the positions described above. Further included within the scope of the application are KARI enzymes, other than the Shewanella sp. KARI (SEQ ID NO: 2), which contain modifications or mutations corresponding to those set out above. In an exemplary embodiment, the modified KARI is derived from a KARI that is at least about 80% identical to SEQ ID NO: 2.

Additional mutated ketol-acid reductoisomerase (KARI) enzymes that utilize NADH rather than NADPH include enzymes having one or more modifications or mutations at positions corresponding to amino acids selected from: (a) valine 48 of the L. lactis KARI (SEQ ID NO: 10); (b) arginine 49 of the L. lactis KARI (SEQ ID NO: 10); (c) lysine 52 of the L. lactis KARI (SEQ ID NO: 10); (d) serine 53 of the L. lactis KARI (SEQ ID NO: 10); (e) glutamic acid 59 of the L. lactis KARI (SEQ ID NO: 10): (f); leucine 85 of the L. lactis KARI (SEQ ID NO: 10); (g) isoleucine 89 of the L. lactis KARI (SEQ ID NO: 10); (h) lysine 118 of the L. lactis KARI (SEQ ID NO: 10); (i) threonine 182 of the L. lactis KARI (SEQ ID NO: 10); and (j) glutamic acid 320 of the L. lactis KARI (SEQ ID NO: 10).

In one embodiment, the KARI enzyme contains a modification or mutation at the amino acid corresponding to position 48 of the L. lactis KARI (SEQ ID NO: 10). In another embodiment the KARI enzyme contains a modification or mutation at the amino acid corresponding to position 49 of the L. lactis KARI (SEQ ID NO: 10). In yet another embodiment, the KARI enzyme contains a modification or mutation at the amino acid corresponding to position 52 of the L. lactis KARI (SEQ ID NO: 10). In yet another embodiment, the KARI enzyme contains a modification or mutation at the amino acid corresponding to position 53 of the L. lactis KARI (SEQ ID NO: 10). In yet another embodiment, the KARI enzyme contains a modification or mutation at the amino acid corresponding to position 59 of the L. lactis KARI (SEQ ID NO: 10). In yet another embodiment, the KARI enzyme contains a modification or mutation at the amino acid corresponding to position 85 of the L. lactis KARI (SEQ ID NO: 10). In yet another embodiment, the KARI enzyme contains a modification or mutation at the amino acid corresponding to position 89 of the L. lactis KARI (SEQ ID NO: 10). In yet another embodiment, the KARI enzyme contains a modification or mutation at the amino acid corresponding to position 118 of the L. lactis KARI (SEQ ID NO: 10). In yet another embodiment, the KARI enzyme contains a modification or mutation at the amino acid corresponding to position 182 of the L. lactis KARI (SEQ ID NO: 10). In yet another embodiment, the KARI enzyme contains a modification or mutation at the amino acid corresponding to position 320 of the L. lactis KARI (SEQ ID NO: 10). In one embodiment, the KARI enzyme contains two or more modifications or mutations at the amino acids corresponding to the positions described above. In another embodiment, the KARI enzyme contains three or more modifications or mutations at the amino acids corresponding to the positions described above. In yet another embodiment, the KARI enzyme contains four or more modifications or mutations at the amino acids corresponding to the positions described above. In yet another embodiment, the KARI enzyme contains five or more modifications or mutations at the amino acids corresponding to the positions described above. In yet another embodiment, the KARI enzyme contains six or more modifications or mutations at the amino acids corresponding to the positions described above. In yet another embodiment, the KARI enzyme contains seven or more modifications or mutations at the amino acids corresponding to the positions described above. In yet another embodiment, the KARI enzyme contains eight or more modifications or mutations at the amino acids corresponding to the positions described above. In yet another embodiment, the KARI enzyme contains nine or more modifications or mutations at the amino acids corresponding to the positions described above. In yet another embodiment, the KARI enzyme contains ten modifications or mutations at the amino acids corresponding to the positions described above. Further included within the scope of the application are KARI enzymes, other than the L. lactis KARI (SEQ ID NO: 10), which contain modifications or mutations corresponding to those set out above. In an exemplary embodiment, the modified KARI is derived from a KARI that is at least about 80% identical to SEQ ID NO: 10.

Additional mutated ketol-acid reductoisomerase (KARI) enzymes that utilize NADH rather than NADPH include enzymes having one or more modifications or mutations at positions corresponding to amino acids selected from: (a) alanine 71 of the S. enterica KARI (SEQ ID NO: 64); (b) arginine 76 of the S. enterica KARI (SEQ ID NO: 64); (c) serine 78 of the S. enterica KARI (SEQ ID NO: 64); (d) glutamine 110 of the S. enterica KARI (SEQ ID NO: 64); (e) aspartic acid 146 of the S. enterica KARI (SEQ ID NO: 64); (f) glycine 185 of the S. enterica KARI (SEQ ID NO: 64); and (g) lysine 433 of the S. enterica KARI (SEQ ID NO: 64).

In one embodiment, the KARI enzyme contains a modification or mutation at the amino acid corresponding to position 71 of the S. enterica KARI (SEQ ID NO: 64). In another embodiment, the KARI enzyme contains a modification or mutation at the amino acid corresponding to position 76 of the S. enterica KARI (SEQ ID NO: 64). In yet another embodiment, the KARI enzyme contains a modification or mutation at the amino acid corresponding to position 78 of the S. enterica KARI (SEQ ID NO: 64). In yet another embodiment, the KARI enzyme contains a modification or mutation at the amino acid corresponding to position 110 of the S. enterica KARI (SEQ ID NO: 64). In yet another embodiment, the KARI enzyme contains a modification or mutation at the amino acid corresponding to position 146 of the S. enterica KARI (SEQ ID NO: 64). In yet another embodiment, the KARI enzyme contains a modification or mutation at the amino acid corresponding to position 185 of the S. enterica KARI (SEQ ID NO: 64). In yet another embodiment, the KARI enzyme contains a modification or mutation at the amino acid corresponding to position 433 of the S. enterica KARI (SEQ ID NO: 64). In one embodiment, the KARI enzyme contains two or more modifications or mutations at the amino acids corresponding to the positions described above. In another embodiment, the KARI enzyme contains three or more modifications or mutations at the amino acids corresponding to the positions described above. In yet another embodiment, the KARI enzyme contains four or more modifications or mutations at the amino acids corresponding to the positions described above. In yet another embodiment, the KARI enzyme contains five or more modifications or mutations at the amino acids corresponding to the positions described above. In yet another embodiment, the KARI enzyme contains six or more modifications or mutations at the amino acids corresponding to the positions described above. In yet another embodiment, the KARI enzyme contains seven modifications or mutations at the amino acids corresponding to the positions described above. Further included within the scope of the application are KARI enzymes, other than the S. enterica KARI (SEQ ID NO: 64), which contain modifications or mutations corresponding to those set out above. In an exemplary embodiment, the modified KARI is derived from a KARI that is at least about 99% identical to SEQ ID NO: 64.

In various embodiments described herein, the modified or mutated KARI may exhibit an increased catalytic efficiency with NADH as compared to the wild-type KARI. In one embodiment, the KARI has at least about a 5% increased catalytic efficiency with NADH as compared to the wild-type KARI. In another embodiment, the KARI has at least about a 25%, at least about a 50%, at least about a 75%, at least about a 100%, at least about a 500%, at least about 1000%, or at least about a 10000% increased catalytic efficiency with NADH as compared to the wild-type KARI.

In various embodiments described herein, the modified or mutated KARI may exhibit a decreased Michaelis Menten constant (K_{M}) for NADH as compared to the wild-type KARI. In one embodiment, the KARI has at least about a 5% decreased K_{M} for NADH as compared to the wild-type KARI. In another embodiment, the KARI has at least about a 25%, at least about a 50%, at least about a 75%, at least about a 90%, at least about a 95%, or at least about a 97.5% decreased K_{M} for NADH as compared to the wild-type KARI.

In various embodiments described herein, the modified or mutated KARI may exhibit an increased catalytic constant (k_{cat}) with NADH as compared to the wild-type KARI. In one embodiment, the KARI has at least about a 5% increased k_{cat} with NADH as compared to the wild-type KARI. In another embodiment, the KARI has at least about a 25%, at least about a 50%, at least about a 75%, at least about 100%, at least about 200%, or at least about a 500% increased k_{cat} with NADH as compared to the wild-type KARI.

In various embodiments described herein, the modified or mutated KARI may exhibit an increased Michaelis Menten constant (K_{M}) for NADPH as compared to the wild-type KARI. In one embodiment, the KARI has at least about a 5% increased K_{M} for NADPH as compared to the wild-type KARI. In another embodiment, the KARI has at least about a 25%, at least about a 50%, at least about a 100%, at least about a 500%, at least about a 1000%, or at least about a 5000% increased K_{M} for NADPH as compared to the wild-type KARI.

In various embodiments described herein, the modified or mutated KARI may exhibit a decreased catalytic constant (k_{cat}) with NADPH as compared to the wild-type KARI. In one embodiment, the KARI has at least about a 5% decreased k_{cat} with NADPH as compared to the wild-type KARI. In another embodiment, the KARI has at least about a 25%, at least about a 50%, or at least about a 75%, at least about 90% decreased k_{cat} with NADPH as compared to the wild-type KARI.

In some embodiments described herein, the catalytic efficiency of the modified or mutated KARI with NADH is increased with respect to the catalytic efficiency with NADPH of the wild-type KARI. In one embodiment, the catalytic efficiency of said KARI with NADH is at least about 10% of the catalytic efficiency with NADPH of the wild-type KARI. In another embodiment, the catalytic efficiency of said KARI with NADH is at least about 25%, at least about 50%, or at least about 75% of the catalytic efficiency with NADPH of the wild-type KARI. In some embodiments, the modified or mutated KARI preferentially utilizes NADH rather than NADPH.

In one embodiment, the application is directed to NADH-dependent KARI enzymes having a catalytic efficiency with NADH that is greater than the catalytic efficiency with NADPH. In one embodiment, the catalytic efficiency of the NADH-dependent KARI is at least about 2-fold greater with NADH than with NADPH. In another embodiment, the catalytic efficiency of the NADH-dependent KARI is at least about 4-fold, at least about 5-fold, at least about 6-fold, at least about 7-fold, at least about 8-fold, at least about 9-fold, at least about 10-fold, at least about 25-fold, at least about 50-fold, at least about 100-fold, or at least about 500-fold greater with NADH than with NADPH.

In one embodiment, the application is directed to modified or mutated KARI enzymes that demonstrate a switch in cofactor specificity from NADPH to NADH. In one embodiment, the modified or mutated KARI has at least about a 2:1 ratio of catalytic efficiency (k_{cat}/K_{M}) with NADH over k_{cat} with NADPH. In an exemplary embodiment, the modified or mutated KARI has at least about a 10:1 ratio of catalytic efficiency (k_{cat}/K_{M}) with NADH over catalytic efficiency (k_{cat}/K_{M}) with NADPH.

In one embodiment, the KARI exhibits at least about a 1:10 ratio of K_{M} for NADH over K_{M} for NADPH.

In additional embodiments, the application is directed to modified or mutated KARI enzymes that have been codon optimized for expression in certain desirable host organisms, such as yeast and E. coli.

In another aspect, the application relates to a recombinant microorganism comprising at least one nucleic acid molecule encoding a KARI enzyme. In one embodiment, the nucleic acid molecule encodes a KARI that is at least about 80% identical to SEQ ID NO: 2, SEQ ID NO: 4, SEQ ID NO: 6, SEQ ID NO: 8, SEQ ID NO: 10, SEQ ID NO: 28, SEQ ID NO: 40, or SEQ ID NO: 58. In another embodiment, the nucleic acid molecule encodes a KARI this is at least about 99% identical to SEQ ID NO: 64. In yet another embodiment, the nucleic acid molecule encodes an NADH-dependent ketol-acid reductoisomerase (NKR) derived from a KARI that is at least about 80% identical to SEQ ID NO: 2, SEQ ID NO: 4, SEQ ID NO: 6, SEQ ID NO: 8, SEQ ID NO: 10, SEQ ID NO: 28, SEQ ID NO: 40, or SEQ ID NO: 58. In one embodiment, the nucleic acid molecule encodes an NADH-dependent ketol-acid reductoisomerase (NKR) derived from a KARI that is at least about 80% identical to SEQ ID NO: 2, wherein said NKR has one or more modifications or mutations at positions corresponding to amino acids selected from: (a) alanine 71 of the Shewanella sp. KARI (SEQ ID NO: 2); (b) arginine 76 of the Shewanella sp. KARI (SEQ ID NO: 2): (c) serine 78 of the Shewanella sp. KARI; and (d) glutamine 110 of the Shewanella sp. KARI (SEQ ID NO: 2). In another embodiment, the nucleic acid molecule encodes an NADH-dependent ketol-acid reductoisomerase (NKR) derived from a KARI that is at least about 80% identical to SEQ ID NO: 10, wherein said NKR has one or more modifications or mutations at positions corresponding to amino acids selected from: (a) valine 48 of the L. lactis KARI (SEQ ID NO: 10); (b) arginine 49 of the L. lactis KARI (SEQ ID NO: 10); (c) lysine 52 of the L. lactis KARI (SEQ ID NO: 10); (d) serine 53 of the L. lactis KARI (SEQ ID NO: 10); (e) glutamic acid 59 of the L. lactis KARI (SEQ ID NO: 10): (f); leucine 85 of the L. lactis KARI (SEQ ID NO: 10); (g) isoleucine 89 of the L. lactis KARI (SEQ ID NO: 10); (h) lysine 118 of the L. lactis KARI (SEQ ID NO: 10); (i) threonine 182 of the L. lactis KARI (SEQ ID NO: 10); and (j) glutamic acid 320 of the L. lactis KARI (SEQ ID NO: 10). In one embodiment, the nucleic acid molecule encodes an NADH-dependent ketol-acid reductoisomerase (NKR) derived from a KARI that is at least about 99% identical to SEQ ID NO: 64. In a further embodiment, the nucleic acid molecule encodes an NADH-dependent ketol-acid reductoisomerase (NKR) derived from a KARI that is at least about 99% identical to SEQ ID NO: 64, wherein said NKR has one or more modifications or mutations at positions corresponding to amino acids selected from: (a) alanine 71 of the S. enterica KARI (SEQ ID NO: 64); (b) arginine 76 of the S. enterica KARI (SEQ ID NO: 64); (c) serine 78 of the S. enterica KARI (SEQ ID NO: 64); (d) glutamine 110 of the S. enterica KARI (SEQ ID NO: 64); (e) aspartic acid 146 of the S. enterica KARI (SEQ ID NO: 64); (f) glycine 185 of the S. enterica KARI (SEQ ID NO: 64); and (g) lysine 433 of the S. enterica KARI (SEQ ID NO: 64).

In various embodiments described in the application, the recombinant microorganism comprises an isobutanol producing metabolic pathway. In one embodiment, the isobutanol producing metabolic pathway comprises at least one exogenous gene encoding a polypeptide that catalyzes a step in the conversion of pyruvate to isobutanol. In another embodiment, the isobutanol producing metabolic pathway comprises at least two exogenous genes encoding polypeptides that catalyze steps in the conversion of pyruvate to isobutanol. In yet another embodiment, the isobutanol producing metabolic pathway comprises at least three exogenous genes encoding polypeptides that catalyze steps in the conversion of pyruvate to isobutanol. In yet another embodiment, the isobutanol producing metabolic pathway comprises at least four exogenous genes encoding polypeptides that catalyze steps in the conversion of pyruvate to isobutanol. In yet another embodiment, the isobutanol producing metabolic pathway comprises at least five exogenous genes encoding polypeptides that catalyze steps in the conversion of pyruvate to isobutanol. In yet another embodiment, all of the isobutanol producing metabolic pathway steps in the conversion of pyruvate to isobutanol are converted by exogenously encoded enzymes. In an exemplary embodiment, at least one of the exogenously encoded enzymes is a KARI that is at least about 80% identical to SEQ ID NO: 2, SEQ ID NO: 4, SEQ ID NO: 6, SEQ ID NO: 8, SEQ ID NO: 10, SEQ ID NO: 28, SEQ ID NO: 40, or SEQ ID NO: 58. In another exemplary embodiment, at least one of the exogenously encoded enzymes is a KARI that is at least about 99% identical to SEQ ID NO: 64. In yet another exemplary embodiment, at least one of the exogenously encoded enzymes is a KARI enzyme has one or more modifications or mutations at positions corresponding to amino acids selected from: (a) alanine 71 of the Shewanella sp. KARI (SEQ ID NO: 2); (b) arginine 76 of the Shewanella sp. KARI (SEQ ID NO: 2); (c) serine 78 of the Shewanella sp. KARI; and (d) glutamine 110 of the Shewanella sp. KARI (SEQ ID NO: 2). In yet another exemplary embodiment, at least one of the exogenously encoded enzymes is a KARI enzyme has one or more modifications or mutations at positions corresponding to amino acids selected from: (a) valine 48 of the L. lactis KARI (SEQ ID NO: 10); (b) arginine 49 of the L. lactis KARI (SEQ ID NO: 10); (c) lysine 52 of the L. lactis KARI (SEQ ID NO: 10); (d) serine 53 of the L. lactis KARI (SEQ ID NO: 10); (e) glutamic acid 59 of the L. lactis KARI (SEQ ID NO: 10): (f); leucine 85 of the L. lactis KARI (SEQ ID NO: 10); (g) isoleucine 89 of the L. lactis KARI (SEQ ID NO: 10); (h) lysine 118 of the L. lactis KARI (SEQ ID NO: 10); (i) threonine 182 of the L. lactis KARI (SEQ ID NO: 10); and (j) glutamic acid 320 of the L. lactis KARI (SEQ ID NO: 10). In yet another exemplary embodiment, at least one of the exogenously encoded enzymes is a KARI enzyme has one or more modifications or mutations at positions corresponding to amino acids selected from: (a) alanine 71 of the S. enterica KARI (SEQ ID NO: 64); (b) arginine 76 of the S. enterica KARI (SEQ ID NO: 64); (c) serine 78 of the S. enterica KARI (SEQ ID NO: 64): (d) glutamine 110 of the S. enterica KARI (SEQ ID NO: 64); (e) aspartic acid 146 of the S. enterica KARI (SEQ ID NO: 64); (f) glycine 185 of the S. enterica KARI (SEQ ID NO: 64); and (g) lysine 433 of the S. enterica KARI (SEQ ID NO: 64).

In one embodiment, one or more of the isobutanol pathway genes encodes an enzyme that is localized to the cytosol. In one embodiment, the recombinant microorganisms comprise an isobutanol producing metabolic pathway with at least one isobutanol pathway enzyme localized in the cytosol. In another embodiment, the recombinant microorganisms comprise an isobutanol producing metabolic pathway with at least two isobutanol pathway enzymes localized in the cytosol. In yet another embodiment, the recombinant microorganisms comprise an isobutanol producing metabolic pathway with at least three isobutanol pathway enzymes localized in the cytosol. In yet another embodiment, the recombinant microorganisms comprise an isobutanol producing metabolic pathway with at least four isobutanol pathway enzymes localized in the cytosol. In an exemplary embodiment, the recombinant microorganisms comprise an isobutanol producing metabolic pathway with five isobutanol pathway enzymes localized in the cytosol. In yet another exemplary embodiment, the recombinant microorganisms comprise an isobutanol producing metabolic pathway with all isobutanol pathway enzymes localized in the cytosol.

In various embodiments described herein, the isobutanol pathway genes may encode enzyme(s) selected from the group consisting of acetolactate synthase (ALS), ketol-acid reductoisomerase (KARI), dihydroxyacid dehydratase (DHAD), 2-keto-acid decarboxylase, e.g., keto-isovalerate decarboxylase (KIVD), and alcohol dehydrogenase (ADH). In one embodiment, the KARI is an NADH-dependent KARI (NKR). In another embodiment, the ADH is an NADH-dependent ADH. In yet another embodiment, the KARI is an NADH-dependent KARI (NKR) and the ADH is an NADH-dependent ADH. In an exemplary embodiment, the KARI is at least about 80% identical to SEQ ID NO: 2, SEQ ID NO: 4, SEQ ID NO: 6, SEQ ID NO: 8, SEQ ID NO: 10, SEQ ID NO: 28, SEQ ID NO: 40, or SEQ ID NO: 58. In another exemplary embodiment, the KARI is at least about 99% identical to SEQ ID NO: 64. In yet another exemplary embodiment, the KARI comprises one or more modifications or mutations at positions corresponding to amino acids selected from: (a) alanine 71 of the Shewanella sp. KARI (SEQ ID NO: 2); (b) arginine 76 of the Shewanella sp. KARI (SEQ ID NO: 2); (c) serine 78 of the Shewanella sp. KARI; and (d) glutamine 110 of the Shewanella sp. KARI (SEQ ID NO: 2). In yet another exemplary embodiment, the KARI comprises one or more modifications or mutations at positions corresponding to amino acids selected from: (a) valine 48 of the L. lactis KARI (SEQ ID NO: 10); (b) arginine 49 of the L. lactis KARI (SEQ ID NO: 10); (c) lysine 52 of the L. lactis KARI (SEQ ID NO: 10); (d) serine 53 of the L. lactis KARI (SEQ ID NO: 10); (e) glutamic acid 59 of the L. lactis KARI (SEQ ID NO: 10): (f); leucine 85 of the L. lactis KARI (SEQ ID NO: 10); (g) isoleucine 89 of the L. lactis KARI (SEQ ID NO: 10); (h) lysine 118 of the L. lactis KARI (SEQ ID NO: 10); (i) threonine 182 of the L. lactis KARI (SEQ ID NO: 10); and (j) glutamic acid 320 of the L. lactis KARI (SEQ ID NO: 10). In yet another exemplary embodiment, the KARI comprises one or more modifications or mutations at positions corresponding to amino acids selected from: (a) alanine 71 of the S. enterica KARI (SEQ ID NO: 64); (b) arginine 76 of the S. enterica KARI (SEQ ID NO: 64); (c) serine 78 of the S. enterica KARI (SEQ ID NO: 64): (d) glutamine 110 of the S. enterica KARI (SEQ ID NO: 64); (e) aspartic acid 146 of the S. enterica KARI (SEQ ID NO: 64); (f) glycine 185 of the S. enterica KARI (SEQ ID NO: 64); and (g) lysine 433 of the S. enterica KARI (SEQ ID NO: 64).

In various embodiments described herein, the recombinant microorganisms of the invention that comprise an isobutanol producing metabolic pathway may be further engineered to reduce or eliminate the expression or activity of one or more enzymes selected from a pyruvate decarboxylase (PDC), a glycerol-3-phosphate dehydrogenase (GPD), a 3-keto acid reductase (3-KAR), or an aldehyde dehydrogenase (ALDH).

In various embodiments described herein, the recombinant microorganisms may be recombinant yeast microorganisms. In some embodiments, the recombinant yeast microorganisms may be members of the Saccharomyces clade, Saccharomyces sensu stricto microorganisms, Crabtree-negative yeast microorganisms, Crabtree-positive yeast microorganisms, post-WGD (whole genome duplication) yeast microorganisms, pre-WGD (whole genome duplication) yeast microorganisms, and non-fermenting yeast microorganisms.

In some embodiments, the recombinant microorganisms may be yeast recombinant microorganisms of the Saccharomyces clade.

In some embodiments, the recombinant microorganisms may be Saccharomyces sensu stricto microorganisms. In one embodiment, the Saccharomyces sensu stricto is selected from the group consisting of S. cerevisiae, S. kudriavzevii, S. mikatae, S. bayanus, S. uvarum, S. carocanis and hybrids thereof.

In some embodiments, the recombinant microorganisms may be Crabtree-negative recombinant yeast microorganisms. In one embodiment, the Crabtree-negative yeast microorganism is classified into a genera selected from the group consisting of Saccharomyces, Kluyveromyces, Pichia, Issatchenkia, Hansenula, or Candida. In additional embodiments, the Crabtree-negative yeast microorganism is selected from Saccharomyces kluyveri, Kluyveromyces lactis, Kluyveromyces marxianus, Pichia anomala, Pichia stipitis, Hansenula anomala, Candida utilis and Kluyveromyces waltii.

In some embodiments, the recombinant microorganisms may be Crabtree-positive recombinant yeast microorganisms. In one embodiment, the Crabtree-positive yeast microorganism is classified into a genera selected from the group consisting of Saccharomyces, Kluyveromyces, Zygosaccharomyces, Debaryomyces, Candida, Pichia and Schizosaccharomyces. In additional embodiments, the Crabtree-positive yeast microorganism is selected from the group consisting of Saccharomyces cerevisiae, Saccharomyces uvarum, Saccharomyces bayanus, Saccharomyces paradoxus, Saccharomyces castelli, Kluyveromyces thermotolerans, Candida glabrata, Z. bailli, Z. rouxii, Debaryomyces hansenii, Pichia pastorius, Schizosaccharomyces pombe, and Saccharomyces uvarum.

In some embodiments, the recombinant microorganisms may be post-WGD (whole genome duplication) yeast recombinant microorganisms. In one embodiment, the post-WGD yeast recombinant microorganism is classified into a genera selected from the group consisting of Saccharomyces or Candida. In additional embodiments, the post-WGD yeast is selected from the group consisting of Saccharomyces cerevisiae, Saccharomyces uvarum, Saccharomyces bayanus, Saccharomyces paradoxus, Saccharomyces castelli, and Candida glabrata.

In some embodiments, the recombinant microorganisms may be pre-WGD (whole genome duplication) yeast recombinant microorganisms. In one embodiment, the pre-WGD yeast recombinant microorganism is classified into a genera selected from the group consisting of Saccharomyces, Kluyveromyces, Candida, Pichia, Issatchenkia, Debaryomyces, Hansenula, Pachysolen, Yarrowia and Schizosaccharomyces. In additional embodiments, the pre-WGD yeast is selected from the group consisting of Saccharomyces kluyveri, Kluyveromyces thermotolerans, Kluyveromyces marxianus, Kluyveromyces waltii, Kluyveromyces lactis, Candida tropicalis, Pichia pastoris, Pichia anomala, Pichia stipitis, Issatchenkia orientalis, Issatchenkia occidentalis, Debaryomyces hansenii, Hansenula anomala, Pachysolen tannophilis, Yarrowia lipolytica, and Schizosaccharomyces pombe.

In some embodiments, the recombinant microorganisms may be microorganisms that are non-fermenting yeast microorganisms, including, but not limited to those, classified into a genera selected from the group consisting of Tricosporon, Rhodotorula, Myxozyma, or Candida. In a specific embodiment, the non-fermenting yeast is C. xestobii.

In another aspect, the present invention provides methods of producing isobutanol using a recombinant microorganism as described herein. In one embodiment, the method includes cultivating the recombinant microorganism in a culture medium containing a feedstock providing the carbon source until a recoverable quantity of isobutanol is produced and optionally, recovering the isobutanol. In one embodiment, the microorganism produces isobutanol from a carbon source at a yield of at least about 5 percent theoretical. In another embodiment, the microorganism produces isobutanol at a yield of at least about 10 percent, at least about 15 percent, about least about 20 percent, at least about 25 percent, at least about 30 percent, at least about 35 percent, at least about 40 percent, at least about 45 percent, at least about 50 percent, at least about 55 percent, at least about 60 percent, at least about 65 percent, at least about 70 percent, at least about 75 percent, at least about 80 percent, at least about 85 percent, at least about 90 percent, at least about 95 percent, or at least about 97.5 percent theoretical.

In one embodiment, the recombinant microorganism converts the carbon source to isobutanol under aerobic conditions. In another embodiment, the recombinant microorganism converts the carbon source to isobutanol under microaerobic conditions. In yet another embodiment, the recombinant microorganism converts the carbon source to isobutanol under anaerobic conditions.

### BRIEF DESCRIPTION OF DRAWINGS

Illustrative embodiments of the invention are illustrated in the drawings, in which:

FIG. 1 illustrates an exemplary embodiment of an isobutanol pathway.

FIG. 2 illustrates an exemplary embodiment of an NADH-dependent isobutanol pathway.

### DETAILED DESCRIPTION

As used herein and in the appended claims, the singular forms “a,” “an,” and “the” include plural referents unless the context clearly dictates otherwise. Thus, for example, reference to “a polynucleotide” includes a plurality of such polynucleotides and reference to “the microorganism” includes reference to one or more microorganisms, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Any publications discussed above and throughout the text are provided solely for their disclosure prior to the filing date of the present application. Nothing herein is to be construed as an admission that the inventors are not entitled to antedate such disclosure by virtue of prior disclosure.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophiles (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consist mainly of hyperthermophilic sulfur-dependent prokaryotes and the Euryarchaeota contain the methanogens and extreme halophiles.

“Bacteria”, or “eubacteria”, refers to a domain of prokaryotic organisms. Bacteria include at least eleven distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

The term “genus” is defined as a taxonomic group of related species according to the Taxonomic Outline of Bacteria and Archaea (Garrity, G. M., Lilbum, T. G., Cole, J. R., Harrison, S. H., Euzeby, J., and Tindall, B. J. (2007) The Taxonomic Outline of Bacteria and Archaea. TOBA Release 7.7, March 2007. Michigan State University Board of Trustees.

The term “species” is defined as a collection of closely related organisms with greater than 97% 16S ribosomal RNA sequence homology and greater than 70% genomic hybridization and sufficiently different from all other organisms so as to be recognized as a distinct unit.

The terms “recombinant microorganism,” “modified microorganism,” and “recombinant host cell” are used interchangeably herein and refer to microorganisms that have been genetically modified to express or to overexpress endogenous polynucleotides, to express heterologous polynucleotides, such as those included in a vector, in an integration construct, or which have an alteration in expression of an endogenous gene. By “alteration” it is meant that the expression of the gene, or level of a RNA molecule or equivalent RNA molecules encoding one or more polypeptides or polypeptide subunits, or activity of one or more polypeptides or polypeptide subunits is up regulated or down regulated, such that expression, level, or activity is greater than or less than that observed in the absence of the alteration. For example, the term “alter” can mean “inhibit,” but the use of the word “alter” is not limited to this definition. It is understood that the terms “recombinant microorganism” and “recombinant host cell” refer not only to the particular recombinant microorganism but to the progeny or potential progeny of such a microorganism. Because certain modifications may occur in succeeding generations due to either mutation or environmental influences, such progeny may not, in fact, be identical to the parent cell, but are still included within the scope of the term as used herein.

The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence. The level of expression of a desired product in a host cell may be determined on the basis of either the amount of corresponding mRNA that is present in the cell, or the amount of the desired product encoded by the selected sequence. For example, mRNA transcribed from a selected sequence can be quantitated by qRT-PCR or by Northern hybridization (see Sambrook et al., Molecular Cloning: A Laboratory Manual, Cold Spring Harbor Laboratory Press (1989)). Protein encoded by a selected sequence can be quantitated by various methods, e.g., by ELISA, by assaying for the biological activity of the protein, or by employing assays that are independent of such activity, such as western blotting or radioimmunoassay, using antibodies that recognize and bind the protein. See Sambrook et al., 1989, supra.

The term “overexpression” refers to an elevated level (e.g., aberrant level) of mRNAs encoding for a protein(s), and/or to elevated levels of protein(s) in cells as compared to similar corresponding unmodified cells expressing basal levels of mRNAs or having basal levels of proteins. In particular embodiments, mRNA(s) or protein(s) may be overexpressed by at least 2-fold, 3-fold, 4-fold, 5-fold, 6-fold, 8-fold, 10-fold, 12-fold, 15-fold or more in microorganisms engineered to exhibit increased gene mRNA, protein, and/or activity.

As used herein and as would be understood by one of ordinary skill in the art, “reduced activity and/or expression” of a protein such as an enzyme can mean either a reduced specific catalytic activity of the protein (e.g. reduced activity) and/or decreased concentrations of the protein in the cell (e.g. reduced expression). As would be understood by one or ordinary skill in the art, the reduced activity of a protein in a cell may result from decreased concentrations of the protein in the cell.

The term “wild-type microorganism” describes a cell that occurs in nature, i.e. a cell that has not been genetically modified. A wild-type microorganism can be genetically modified to express or overexpress a first target enzyme. This microorganism can act as a parental microorganism in the generation of a microorganism modified to express or overexpress a second target enzyme. In turn, the microorganism modified to express or overexpress a first and a second target enzyme can be modified to express or overexpress a third target enzyme.

Accordingly, a “parental microorganism” functions as a reference cell for successive genetic modification events. Each modification event can be accomplished by introducing a nucleic acid molecule in to the reference cell. The introduction facilitates the expression or overexpression of a target enzyme. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of heterologous polynucleotides encoding a target enzyme in to a parental microorganism

The term “engineer” refers to any manipulation of a microorganism that results in a detectable change in the microorganism, wherein the manipulation includes but is not limited to inserting a polynucleotide and/or polypeptide heterologous to the microorganism and mutating a polynucleotide and/or polypeptide native to the microorganism.

The term “mutation” as used herein indicates any modification of a nucleic acid and/or polypeptide which results in an altered nucleic acid or polypeptide. Mutations include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, a nonsense mutation, an insertion, or a deletion of part or all of a gene. In addition, in some embodiments of the modified microorganism, a portion of the microorganism genome has been replaced with a heterologous polynucleotide. In some embodiments, the mutations are naturally-occurring. In other embodiments, the mutations are identified and/or enriched through artificial selection pressure. In still other embodiments, the mutations in the microorganism genome are the result of genetic engineering.

The term “biosynthetic pathway”, also referred to as “metabolic pathway”, refers to a set of anabolic or catabolic biochemical reactions for converting one chemical species into another. Gene products belong to the same “metabolic pathway” if they, in parallel or in series, act on the same substrate, produce the same product, or act on or produce a metabolic intermediate (i.e., metabolite) between the same substrate and metabolite end product.

As used herein, the term “isobutanol producing metabolic pathway” refers to an enzyme pathway which produces isobutanol from pyruvate.

The term “NADH-dependent” as used herein with reference to an enzyme, e.g., KARI and/or ADH, refers to an enzyme that catalyzes the reduction of a substrate coupled to the oxidation of NADH with a catalytic efficiency that is greater than the reduction of the same substrate coupled to the oxidation of NADPH at equal substrate and cofactor concentrations.

The term “exogenous” as used herein with reference to various molecules, e.g., polynucleotides, polypeptides, enzymes, etc., refers to molecules that are not normally or naturally found in and/or produced by a given yeast, bacterium, organism, microorganism, or cell in nature.

On the other hand, the term “endogenous” or “native” as used herein with reference to various molecules, e.g., polynucleotides, polypeptides, enzymes, etc., refers to molecules that are normally or naturally found in and/or produced by a given yeast, bacterium, organism, microorganism, or cell in nature.

The term “heterologous” as used herein in the context of a modified host cell refers to various molecules, e.g., polynucleotides, polypeptides, enzymes, etc., wherein at least one of the following is true: (a) the molecule(s) is/are foreign (“exogenous”) to (i.e., not naturally found in) the host cell; (b) the molecule(s) is/are naturally found in (e.g., is “endogenous to”) a given host microorganism or host cell but is either produced in an unnatural location or in an unnatural amount in the cell; and/or (c) the molecule(s) differ(s) in nucleotide or amino acid sequence from the endogenous nucleotide or amino acid sequence(s) such that the molecule differing in nucleotide or amino acid sequence from the endogenous nucleotide or amino acid as found endogenously is produced in an unnatural (e.g., greater than naturally found) amount in the cell.

The term “feedstock” is defined as a raw material or mixture of raw materials supplied to a microorganism or fermentation process from which other products can be made. For example, a carbon source, such as biomass or the carbon compounds derived from biomass are a feedstock for a microorganism that produces a biofuel in a fermentation process. However, a feedstock may contain nutrients other than a carbon source.

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, such as any biomass derived sugar, but also intermediate and end product metabolites used in a pathway associated with a recombinant microorganism as described herein.

The term “fermentation” or “fermentation process” is defined as a process in which a microorganism is cultivated in a culture medium containing raw materials, such as feedstock and nutrients, wherein the microorganism converts raw materials, such as a feedstock, into products.

The term “volumetric productivity” or “production rate” is defined as the amount of product formed per volume of medium per unit of time. Volumetric productivity is reported in gram per liter per hour (g/L/h).

The term “specific productivity” or “specific production rate” is defined as the amount of product formed per volume of medium per unit of time per amount of cells. Specific productivity is reported in gram (or milligram) per gram cell dry weight per hour (g/g h).

The term “yield” is defined as the amount of product obtained per unit weight of raw material and may be expressed as g product per g substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product. For example, the theoretical yield for one typical conversion of glucose to isobutanol is 0.41 g/g. As such, a yield of isobutanol from glucose of 0.39 gig would be expressed as 95% of theoretical or 95% theoretical yield.

The term “titer” is defined as the strength of a solution or the concentration of a substance in solution. For example, the titer of a biofuel in a fermentation broth is described as g of biofuel in solution per liter of fermentation broth (g/L).

“Aerobic conditions” are defined as conditions under which the oxygen concentration in the fermentation medium is sufficiently high for an aerobic or facultative anaerobic microorganism to use as a terminal electron acceptor.

In contrast, “anaerobic conditions” are defined as conditions under which the oxygen concentration in the fermentation medium is too low for the microorganism to use as a terminal electron acceptor. Anaerobic conditions may be achieved by sparging a fermentation medium with an inert gas such as nitrogen until oxygen is no longer available to the microorganism as a terminal electron acceptor. Alternatively, anaerobic conditions may be achieved by the microorganism consuming the available oxygen of the fermentation until oxygen is unavailable to the microorganism as a terminal electron acceptor. Methods for the production of isobutanol under anaerobic conditions are described in commonly owned and co-pending publication, US 2010/0143997, the disclosures of which are herein incorporated by reference in its entirety for all purposes.

“Aerobic metabolism” refers to a biochemical process in which oxygen is used as a terminal electron acceptor to make energy, typically in the form of ATP, from carbohydrates. Aerobic metabolism occurs, e.g., via glycolysis and the TCA cycle, wherein a single glucose molecule is metabolized completely into carbon dioxide in the presence of oxygen.

In contrast, “anaerobic metabolism” refers to a biochemical process in which oxygen is not the final acceptor of electrons contained in NADH. Anaerobic metabolism can be divided into anaerobic respiration, in which compounds other than oxygen serve as the terminal electron acceptor, and substrate level phosphorylation, in which the electrons from NADH are utilized to generate a reduced product via a “fermentative pathway.”

In “fermentative pathways”, NAD(P)H donates its electrons to a molecule produced by the same metabolic pathway that produced the electrons carried in NAD(P)H. For example, in one of the fermentative pathways of certain yeast strains, NAD(P)H generated through glycolysis transfers its electrons to pyruvate, yielding ethanol. Fermentative pathways are usually active under anaerobic conditions but may also occur under aerobic conditions, under conditions where NADH is not fully oxidized via the respiratory chain. For example, above certain glucose concentrations, Crabtree positive yeasts produce large amounts of ethanol under aerobic conditions.

The term “byproduct” or “by-product” means an undesired product related to the production of an amino acid, amino acid precursor, chemical, chemical precursor, biofuel, biofuel precursor, higher alcohol, or higher alcohol precursor.

The term “substantially free” when used in reference to the presence or absence of a protein activity (3-KAR enzymatic activity, ALDH enzymatic activity, PDC enzymatic activity. GPD enzymatic activity, etc.) means the level of the protein is substantially less than that of the same protein in the wild-type host, wherein less than about 50% of the wild-type level is preferred and less than about 30% is more preferred. The activity may be less than about 20%, less than about 10%, less than about 5%, or less than about 1% of wild-type activity. Microorganisms which are “substantially free” of a particular protein activity (3-KAR enzymatic activity, ALDH enzymatic activity, PDC enzymatic activity, GPD enzymatic activity, etc.) may be created through recombinant means or identified in nature.

The term “non-fermenting yeast” is a yeast species that fails to demonstrate an anaerobic metabolism in which the electrons from NADH are utilized to generate a reduced product via a fermentative pathway such as the production of ethanol and CO₂ from glucose. Non-fermentative yeast can be identified by the “Durham Tube Test” (J. A. Bamett, R. W. Payne, and D. Yarrow. 2000. Yeasts Characteristics and Identification. 3^{rd} edition. p. 28-29. Cambridge University Press, Cambridge, UK) or by monitoring the production of fermentation productions such as ethanol and CO₂.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, DNA, RNA, analogs and fragments thereof. A polynucleotide of three or more nucleotides is also called nucleotidic oligomer or oligonucleotide.

It is understood that the polynucleotides described herein include “genes” and that the nucleic acid molecules described herein include “vectors” or “plasmids.” Accordingly, the term “gene”, also called a “structural gene” refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence.

The term “operon” refers to two or more genes which are transcribed as a single transcriptional unit from a common promoter. In some embodiments, the genes comprising the operon are contiguous genes. It is understood that transcription of an entire operon can be modified (i.e., increased, decreased, or eliminated) by modifying the common promoter. Alternatively, any gene or combination of genes in an operon can be modified to alter the function or activity of the encoded polypeptide. The modification can result in an increase in the activity of the encoded polypeptide. Further, the modification can impart new activities on the encoded polypeptide. Exemplary new activities include the use of alternative substrates and/or the ability to function in alternative environmental conditions.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

“Transformation” refers to the process by which a vector is introduced into a host cell. Transformation (or transduction, or transfection), can be achieved by any one of a number of means including chemical transformation (e.g. lithium acetate transformation), electroporation, microinjection, biolistics (or particle bombardment-mediated delivery), or agrobacterium mediated transformation.

The term “enzyme” as used herein refers to any substance that catalyzes or promotes one or more chemical or biochemical reactions, which usually includes enzymes totally or partially composed of a polypeptide or polypeptides, but can include enzymes composed of a different molecule including polynucleotides.

The term “protein,” “peptide,” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. The term “amino acid analog” refers to an amino acid in which one or more individual atoms have been replaced, either with a different atom, or with a different functional group. Accordingly, the term polypeptide includes amino acidic polymer of any length including full length proteins, and peptides as well as analogs and fragments thereof. A polypeptide of three or more amino acids is also called a protein oligomer or oligopeptide

The term “homolog,” used with respect to an original polynucleotide or polypeptide of a first family or species, refers to distinct polynucleotides or polypeptides of a second family or species which are determined by functional, structural or genomic analyses to be a polynucleotide or polypeptide of the second family or species which corresponds to the original polynucleotide or polypeptide of the first family or species. Most often, homologs will have functional, structural or genomic similarities. Techniques are known by which homologs of a polynucleotide or polypeptide can readily be cloned using genetic probes and PCR. Identity of cloned sequences as homolog can be confirmed using functional assays and/or by genomic mapping of the genes.

A polypeptide has “homology” or is “homologous” to a second polypeptide if the amino acid sequence encoded by a gene has a similar amino acid sequence to that of the second gene. Alternatively, a polypeptide has homology to a second polypeptide if the two polypeptides have “similar” amino acid sequences. (Thus, the terms “homologous polypeptides” or “homologous proteins” are defined to mean that the two polypeptides have similar amino acid sequences).

The term “analog” or “analogous” refers to polynucleotide or polypeptide sequences that are related to one another in function only and are not from common descent or do not share a common ancestral sequence. Analogs may differ in sequence but may share a similar structure, due to convergent evolution. For example, two enzymes are analogs or analogous if the enzymes catalyze the same reaction of conversion of a substrate to a product, are unrelated in sequence, and irrespective of whether the two enzymes are related in structure.

### Isobutanol Producing Recombinant Microorganisms

A variety of microorganisms convert sugars to produce pyruvate, which is then utilized in a number of pathways of cellular metabolism. In recent years, microorganisms, including yeast, have been engineered to produce a number of desirable products via pyruvate-driven biosynthetic pathways, including isobutanol, an important commodity chemical and biofuel candidate (See, e.g., commonly owned and co-pending patent publications, US 2009/0226991, US 2010/0143997, US 2011/0020889, US 2011/0076733, and WO 2010/075504).

As described herein, the present invention relates to recombinant microorganisms for producing isobutanol, wherein said recombinant microorganisms comprise an isobutanol producing metabolic pathway. In one embodiment, the isobutanol producing metabolic pathway to convert pyruvate to isobutanol can be comprised of the following reactions:

1. 2 pyruvate→acetolactate+CO₂

2. acetolactate+NAD(P)H→2,3-dihydroxyisovalerate+NAD(P)⁺

3. 2,3-dihydroxyisovalerate→alpha-ketoisovalerate

4. alpha-ketoisovalerate→isobutyraldehyde+CO₂

5. isobutyraldehyde+NAD(P)H→isobutanol+NADP

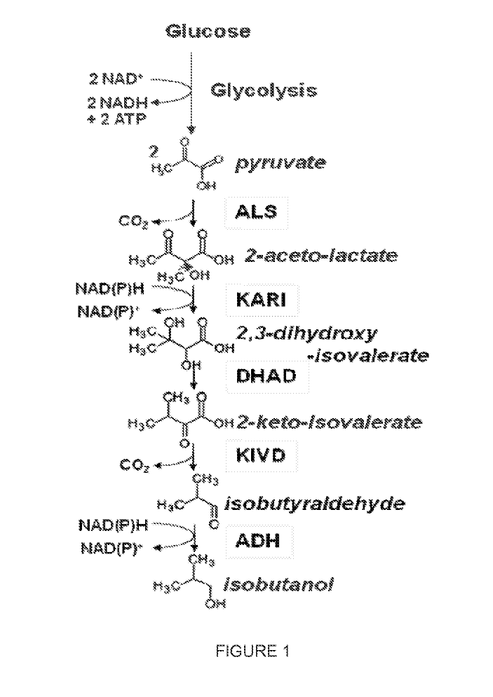

In one embodiment, these reactions are carried out by the enzymes 1) Acetolactate synthase (ALS), 2) Ketol-acid reductoisomerase (KARI), 3) Dihydroxy-acid dehydratase (DHAD), 4) 2-keto-acid decarboxylase, e.g., Keto-isovalerate decarboxylase (KIVD), and 5) an Alcohol dehydrogenase (ADH) (FIG. 1). In some embodiments, the recombinant microorganism may be engineered to overexpress one or more of these enzymes. In an exemplary embodiment, the recombinant microorganism is engineered to overexpress all of these enzymes.

Alternative pathways for the production of isobutanol in yeast have been described in WO/2007/050671 and in Dickinson et al., 1998, J Biol Chem 273:25751-6. These and other isobutanol producing metabolic pathways are within the scope of the present application. In one embodiment, the isobutanol producing metabolic pathway comprises five substrate to product reactions. In another embodiment, the isobutanol producing metabolic pathway comprises six substrate to product reactions. In yet another embodiment, the isobutanol producing metabolic pathway comprises seven substrate to product reactions.

In various embodiments described herein, the recombinant microorganism comprises an isobutanol producing metabolic pathway. In one embodiment, the isobutanol producing metabolic pathway comprises at least one exogenous gene encoding a polypeptide that catalyzes a step in the conversion of pyruvate to isobutanol. In another embodiment, the isobutanol producing metabolic pathway comprises at least two exogenous genes encoding polypeptides that catalyze steps in the conversion of pyruvate to isobutanol. In yet another embodiment, the isobutanol producing metabolic pathway comprises at least three exogenous genes encoding polypeptides that catalyze steps in the conversion of pyruvate to isobutanol. In yet another embodiment, the isobutanol producing metabolic pathway comprises at least four exogenous genes encoding polypeptides that catalyze steps in the conversion of pyruvate to isobutanol. In yet another embodiment, the isobutanol producing metabolic pathway comprises at least five exogenous genes encoding polypeptides that catalyze steps in the conversion of pyruvate to isobutanol. In yet another embodiment, all of the isobutanol producing metabolic pathway steps in the conversion of pyruvate to isobutanol are converted by exogenously encoded enzymes.

In one embodiment, one or more of the isobutanol pathway genes encodes an enzyme that is localized to the cytosol. In one embodiment, the recombinant microorganisms comprise an isobutanol producing metabolic pathway with at least one isobutanol pathway enzyme localized in the cytosol. In another embodiment, the recombinant microorganisms comprise an isobutanol producing metabolic pathway with at least two isobutanol pathway enzymes localized in the cytosol. In yet another embodiment, the recombinant microorganisms comprise an isobutanol producing metabolic pathway with at least three isobutanol pathway enzymes localized in the cytosol. In yet another embodiment, the recombinant microorganisms comprise an isobutanol producing metabolic pathway with at least four isobutanol pathway enzymes localized in the cytosol. In an exemplary embodiment, the recombinant microorganisms comprise an isobutanol producing metabolic pathway with five isobutanol pathway enzymes localized in the cytosol. In yet another exemplary embodiment, the recombinant microorganisms comprise an isobutanol producing metabolic pathway with all isobutanol pathway enzymes localized in the cytosol. Isobutanol producing metabolic pathways in which one or more genes are localized to the cytosol are described in commonly owned and co-pending U.S. application Ser. No. 12/855,276, which is herein incorporated by reference in its entirety for all purposes.

As is understood in the art, a variety of organisms can serve as sources for the isobutanol pathway enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorpha, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but not limited to, Piromyces spp., Orpinomyces spp., or Neocallimastix spp. Sources of prokaryotic enzymes that are useful include, but not limited to, Escherichia spp., Zymomonas spp., Staphylococcus spp., Bacillus spp., Clostridium spp., Corynebacterium spp., Pseudomonas spp., Lactococcus spp., Enterobacter spp., Streptococcus spp., Salmonella spp., Slackia spp., Cryptobacterium spp., and Eggerthella spp.

In some embodiments, one or more of these enzymes can be encoded by native genes. Alternatively, one or more of these enzymes can be encoded by heterologous genes.

For example, acetolactate synthases capable of converting pyruvate to acetolactate may be derived from a variety of sources (e.g., bacterial, yeast, Archaea, etc.), including B. subtilis (GenBank Accession No. Q04789.3), L. lactis (GenBank Accession No. NP_{—}267340.1), S. mutans (GenBank Accession No. NP_{—}721805.1), K. pneumoniae (GenBank Accession No. ZP_{—}06014957.1), C. glutamicum (GenBank Accession No. P42463.1), E. cloacae (GenBank Accession No. YP_{—}003613611.1), M. maripaludis (GenBank Accession No. ABX01060.1), M. grisea (GenBank Accession No. AAB81248.1), T. stipitatus (GenBank Accession No. XP_{—}002485976.1), or S. cerevisiae ILV2 (GenBank Accession No. NP_{—}013826.1). Additional acetolactate synthases capable of converting pyruvate to acetolactate are described in commonly owned and co-pending US Publication No. 2011/0076733, which is herein incorporated by reference in its entirety. A review article characterizing the biosynthesis of acetolactate from pyruvate via the activity of acetolactate synthases is provided by Chipman et al., 1998, Biochimica et Biophysica Acta 1385: 401-19, which is herein incorporated by reference in its entirety. Chipman et al. provide an alignment and consensus for the sequences of a representative number of acetolactate synthases. Motifs shared in common between the majority of acetolactate synthases include:

(SEQ ID NO: 65)
SGPG(A/C/V)(T/S)N,
(SEQ ID NO: 66)
GX(P/A)GX(V/A/T),
(SEQ ID NO: 67)
GX(Q/G)(T/A)(L/M)G(Y/F/W)(A/G)X(P/G)
(W/A)AX(G/T)(A/V),
and
(SEQ ID NO: 68)
GD(G/A)(G/S/C)F

motifs at amino acid positions corresponding to the 163-169, 240-245, 521-535, and 549-553 residues, respectively, of the S. cerevisiae ILV2. Thus, a protein harboring one or more of these amino acid motifs can generally be expected to exhibit acetolactate synthase activity.

Dihydroxy acid dehydratases capable of converting 2,3-dihydroxyisovalerate to α-ketoisovalerate may be derived from a variety of sources (e.g., bacterial, yeast, Archaea, etc.), including E. coli (GenBank Accession No. YP_{—}026248.1), L. lactis (GenBank Accession No. NP_{—}267379.1), S. mutans (GenBank Accession No. NP_{—}722414.1), M. stadtmanae (GenBank Accession No. YP_{—}448586.1), M. tractuosa (GenBank Accession No. YP_{—}004053736.1), Eubacterium SCB49 (GenBank Accession No. ZP_{—}01890126.1), G. forsetti (GenBank Accession No. YP_{—}862145.1), Y. lipolytica (GenBank Accession No. XP_{—}502180.2), N. crassa (GenBank Accession No. XP_{—}963045.1), or S. cerevisiae ILV3 (GenBank Accession No. NP_{—}012550.1). Additional dihydroxy acid dehydratases capable of 2,3-dihydroxyisovalerate to α-ketoisovalerate are described in commonly owned and co-pending US Publication No. 2011/0076733. Motifs shared in common between the majority of dihydroxy acid dehydratases include:

(SEQ ID NO: 69)
SLXSRXXIA,
(SEQ ID NO: 70)
CDKXXPG,
(SEQ ID NO: 71)
GXCXGXXTAN,
(SEQ ID NO: 72)
GGSTN,
(SEQ ID NO: 73)
GPXGXPGMRXE,
(SEQ ID NO: 74)
ALXTDGRXSG,
and
(SEQ ID NO: 75)
GHXXPEA

motifs at amino acid positions corresponding to the 93-101, 122-128, 193-202, 276-280, 482-491, 509-518, and 526-532 residues, respectively, of the E. coli dihydroxy acid dehydratase encoded by ilvD. Thus, a protein harboring one or more of these amino acid motifs can generally be expected to exhibit dihydroxy acid dehydratase activity.

2-keto-acid decarboxylases capable of converting α-ketoisovalerate to isobutyraldehyde may be derived from a variety of sources (e.g., bacterial, yeast, Archaea, etc.), including L. lactis kivD (GenBank Accession No. YP_{—}003353820.1), E. cloacae (GenBank Accession No. P23234.1), M. smegmatis (GenBank Accession No. A0R480.1), M. tuberculosis (GenBank Accession No. 053865.1), M. avium (GenBank Accession No. Q742Q2.1, A. brasilense (GenBank Accession No. P51852.1), L. lactis kdcA (GenBank Accession No. AAS49166.1), S. epidermidis (GenBank Accession No. NP_{—}765765.1), M. caseolyticus (GenBank Accession No. YP_{—}002560734.1), B. megaterium (GenBank Accession No. YP_{—}003561644.1), S. cerevisiae ARO10 (GenBank Accession No. NP_{—}010668.1), or S. cerevisiae THI3 (GenBank Accession No. CAA98646.1). Additional 2-keto-acid decarboxylases capable of converting α-ketoisovalerate to isobutyraldehyde are described in commonly owned and co-pending US Publication No. 2011/0076733. Motifs shared in common between the majority of 2-keto-acid decarboxylases include:

(SEQ ID NO: 76)
FG(V/I)(P/S)G(D/E)(Y/F),
(SEQ ID NO: 77)
(T/V)T(F/Y)G(V/A)G(E/A)(L/F)(S/N),
(SEQ ID NO: 78)
N(G/A)(L/I/V)AG(S/A)(Y/F)AE,
(SEQ ID NO: 79)
(V/I)(L/I/V)XI(V/T/S)G,
and
(SEQ ID NO: 80)
GDG(S/A)(L/F/A)Q(L/M)T

motifs at amino acid positions corresponding to the 21-27, 70-78, 81-89, 93-98, and 428-435 residues, respectively, of the L. lactis 2-keto-acid decarboxylase encoded by kivD. Thus, a protein harboring one or more of these amino acid motifs can generally be expected to exhibit 2-keto-acid decarboxylase activity.

Alcohol dehydrogenases capable of converting isobutyraldehyde to isobutanol may be derived from a variety of sources (e.g., bacterial, yeast, Archaea, etc.), including L. lactis (GenBank Accession No. YP_{—}003354381), B. cereus (GenBank Accession No. YP_{—}001374103.1), N. meningitidis (GenBank Accession No. CBA03965.1), S. sanguinis (GenBank Accession No. YP_{—}001035842.1), L. brevis (GenBank Accession No. YP_{—}794451.1), B. thuringiensis (GenBank Accession No. ZP_{—}04101989.1), P. acidilactici (GenBank Accession No. ZP_{—}06197454.1), B. subtilis (GenBank Accession No. EHA31115.1), N. crassa (GenBank Accession No. CAB91241.1) or S. cerevisiae ADH6 (GenBank Accession No. NP_{—}014051.1). Additional alcohol dehydrogenases capable of converting isobutyraldehyde to isobutanol are described in commonly owned and co-pending US Publication Nos. 2011/0076733 and 2011/0201072. Motifs shared in common between the majority of alcohol dehydrogenases include:

(SEQ ID NO: 81)
C(H/G)(T/S)D(L/I)H,
(SEQ ID NO: 82)
GHEXXGXV,
(SEQ ID NO: 83)
(L/V)(Q/K/E)(V/I/K)G(D/Q)(R/H)(V/A),
(SEQ ID NO: 84)
CXXCXXC,
(SEQ ID NO: 85)
(C/A)(A/G/D)(G/A)XT(T/V),
and
(SEQ ID NO: 86)
G(L/A/C)G(G/P)(L/I/V)G

motifs at amino acid positions corresponding to the 39-44, 59-66, 76-82, 91-97, 147-152, and 171-176 residues, respectively, of the L. lactis alcohol dehydrogenase encoded by adhA. Thus, a protein harboring one or more of these amino acid motifs can generally be expected to exhibit alcohol dehydrogenase activity.

In another embodiment, the yeast microorganism may be engineered to have increased ability to convert pyruvate to isobutanol. In one embodiment, the yeast microorganism may be engineered to have increased ability to convert pyruvate to isobutyraldehyde. In another embodiment, the yeast microorganism may be engineered to have increased ability to convert pyruvate to keto-isovalerate. In another embodiment, the yeast microorganism may be engineered to have increased ability to convert pyruvate to 2,3-dihydroxyisovalerate. In another embodiment, the yeast microorganism may be engineered to have increased ability to convert pyruvate to acetolactate.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in yeast.

Isobutanol-Producing Metabolic Pathways with Improved KARI Properties

As described herein, the present application provides several KARI enzymes that give high performance when expressed in yeast in the context of isobutanol production. Accordingly, this application describes methods of increasing isobutanol production through the use of recombinant microorganisms comprising KARI enzymes with improved properties for the production of isobutanol.

One aspect of the application is directed to an isolated nucleic acid molecule encoding a ketol-acid reductoisomerase (KARI), wherein said KARI is at least about 80% identical to SEQ ID NO: 2, SEQ ID NO: 4, SEQ ID NO: 6, SEQ ID NO: 8, SEQ ID NO: 10, SEQ ID NO; 28, SEQ ID NO: 40, or SEQ ID NO: 56. Further within the scope of present application are KARIs which are at least about 80%, 85%, 90%, 91%, 92%, 93%, 94%, 95%, 96%, 97%, 98%, or 99% identical to SEQ ID NO: 2, SEQ ID NO: 4, SEQ ID NO: 6. SEQ ID NO: 8, SEQ ID NO: 10, SEQ ID NO; 28, SEQ ID NO: 40, or SEQ ID NO: 56.

In one embodiment, the KARI is derived from the genus Shewanella. In a specific embodiment, the KARI is derived from Shewanella sp. strain MR-4. In another specific embodiment, the isolated nucleic acid molecule is comprised of SEQ ID NO: 1. In another embodiment, the KARI is derived from the genus Vibrio. In a specific embodiment, the KARI is derived from Vibrio fischeri. In another specific embodiment, the isolated nucleic acid molecule is comprised of SEQ ID NO: 3. In yet another embodiment, the KARI is derived from the genus Gramella. In a specific embodiment, the KARI is derived from Gramella forsetii. In another specific embodiment, the isolated nucleic acid molecule is comprised of SEQ ID NO: 5. In yet another embodiment, the KARI is derived from the genus Cytophaga. In a specific embodiment, the KARI is derived from Cytophaga hutchinsonii. In another specific embodiment, the isolated nucleic acid molecule is comprised of SEQ ID NO: 7. In yet another embodiment, the KARI is derived from a genus selected from Lactococcus and Streptococcus. In a specific embodiment, the KARI is derived from Lactococcus lactis, Streptococcus equinus, or Streptococcus infantarius. In another specific embodiment, the KARI is encoded by SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25. In yet another embodiment, the KARI is derived from the genus Methanococcus. In a specific embodiment, the KARI is derived from Methanococcus maripaludis, Methanococcus vannielii, or Methanococcus voltae. In another specific embodiment, the KARI is encoded by SEQ ID NO: 27, SEQ ID NO: 29, SEQ ID NO: 31, SEQ ID NO: 33, SEQ ID NO: 35, or SEQ ID NO: 37. In yet another embodiment, the KARI is derived from a genus selected from Zymomonas, Erythrobacter, Sphingomonas, Sphingobium, and Novosphingobium. In a specific embodiment, the KARI is derived from Zymomonas mobilis, Erythrobacter litoralis, Sphingomonas wittichii, Sphingobium japonicum, Sphingobium chlorophenolicum, or Novosphingobium nitrogenifigens. In another specific embodiment, the KARI is encoded by SEQ ID NO: 39, SEQ ID NO: 41, SEQ ID NO: 43, SEQ ID NO: 45, SEQ ID NO: 47, SEQ ID NO: 49, SEQ ID NO: 51, or SEQ ID NO: 53. In yet another embodiment, the KARI is derived from the genus Bacteroides. In a specific embodiment, the KARI is derived from Bacteroides thetaiotaomicron. In another specific embodiment, the KARI is encoded by SEQ ID NO: 55. In yet another embodiment, the KARI is derived from the genus Schizosaccharomyces. In a specific embodiment, the KARI is derived from Schizosaccharomyces pombe or Schizosaccharomyces japonicus. In another specific embodiment, the KARI is encoded by SEQ ID NO: 57, SEQ ID NO: 59, or SEQ ID NO: 61.

Also included within the scope of this application are isolated KARI enzymes that have been modified to be NADH-dependent. Accordingly, the present application further relates to NADH-dependent ketol-acid reductoisomerases (NKRs) derived from a KARI that is at least about 80% identical to SEQ ID NO: 2, SEQ ID NO: 4, SEQ ID NO: 6, SEQ ID NO: 8, SEQ ID NO: 10, SEQ ID NO: 28, SEQ ID NO: 40, or SEQ ID NO: 58.

Another aspect of the application is directed to an isolated nucleic acid molecule encoding a ketol-acid reductoisomerase (KARI), wherein said KARI is at least about 99% identical to SEQ ID NO: 64. In one embodiment, the KARI is derived from the genus Salmonella. In a specific embodiment, the KARI is derived from Salmonella enterica. In another specific embodiment, the KARI is encoded by SEQ ID NO: 63. The present application further relates to NADH-dependent ketol-acid reductoisomerases (NKRs) derived from a KARI that is at least about 99% identical to SEQ ID NO: 64.

The invention also includes fragments of the disclosed KARI enzymes which comprise at least 50, 100, 150, 200, 250, 300, 350, 400, 450, 500, 550, or 600 amino acid residues and retain one or more activities associated with KARI enzymes. Such fragments may be obtained by deletion mutation, by recombinant techniques that are routine and well-known in the art, or by enzymatic digestion of the KARI enzyme(s) of interest using any of a number of well-known proteolytic enzymes. The invention further includes nucleic acid molecules which encode the above described KARI enzymes and KARI enzyme fragments.

Another aspect of the application relates to a recombinant microorganism comprising at least one nucleic acid molecule encoding a ketol-acid reductoisomerase (KARI), wherein said KARI is at least about 80% identical to SEQ ID NO: 2, SEQ ID NO: 4, SEQ ID NO: 6, SEQ ID NO: 8, SEQ ID NO: 10, SEQ ID NO; 28, SEQ ID NO: 40, or SEQ ID NO: 56. Further within the scope of present application are recombinant microorganism comprising at least one nucleic acid molecule encoding a ketol-acid reductoisomerase (KARI), wherein said KARI is at least about 80%, 85%, 90%, 91%, 92%, 93%, 94%, 95%, 96%, 97%, 98%, or 99% identical to SEQ ID NO: 2, SEQ ID NO: 4, SEQ ID NO: 6, SEQ ID NO: 8, SEQ ID NO: 10, SEQ ID NO; 28, SEQ ID NO: 40, or SEQ ID NO: 56.

In one embodiment, the KARI is derived from the genus Shewanella. In a specific embodiment, the KARI is derived from Shewanella sp. strain MR-4. In another specific embodiment, the isolated nucleic acid molecule is comprised of SEQ ID NO: 1. In another embodiment, the KARI is derived from the genus Vibrio. In a specific embodiment, the KARI is derived from Vibrio fischeri. In another specific embodiment, the isolated nucleic acid molecule is comprised of SEQ ID NO: 3. In yet another embodiment, the KARI is derived from the genus Gramella. In a specific embodiment, the KARI is derived from Gramella forsetii. In another specific embodiment, the isolated nucleic acid molecule is comprised of SEQ ID NO: 5. In yet another embodiment, the KARI is derived from the genus Cytophaga. In a specific embodiment, the KARI is derived from Cytophaga hutchinsonii. In another specific embodiment, the isolated nucleic acid molecule is comprised of SEQ ID NO: 7. In yet another embodiment, the KARI is derived from a genus selected from Lactococcus and Streptococcus. In a specific embodiment, the KARI is derived from Lactococcus lactis, Streptococcus equinus, or Streptococcus infantarius. In another specific embodiment, the KARI is encoded by SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25. In yet another embodiment, the KARI is derived from the genus Methanococcus. In a specific embodiment, the KARI is derived from Methanococcus maripaludis, Methanococcus vannielii, or Methanococcus voltae. In another specific embodiment, the KARI is encoded by SEQ ID NO: 27, SEQ ID NO: 29, SEQ ID NO: 31, SEQ ID NO: 33, SEQ ID NO: 35, or SEQ ID NO: 37. In yet another embodiment, the KARI is derived from a genus selected from Zymomonas, Erythrobacter, Sphingomonas, Sphingobium, and Novosphingobium. In a specific embodiment, the KARI is derived from Zymomonas mobilis, Erythrobacter litoralis, Sphingomonas wittichii, Sphingobium japonicum, Sphingobium chlorophenolicum, or Novosphingobium nitrogenifigens. In another specific embodiment, the KARI is encoded by SEQ ID NO: 39, SEQ ID NO: 41, SEQ ID NO: 43, SEQ ID NO: 45, SEQ ID NO: 47, SEQ ID NO: 49, SEQ ID NO: 51, or SEQ ID NO: 53. In yet another embodiment, the KARI is derived from the genus Bacteroides. In a specific embodiment, the KARI is derived from Bacteroides thetaiotaomicron. In another specific embodiment, the KARI is encoded by SEQ ID NO: 55. In yet another embodiment, the KARI is derived from the genus Schizosaccharomyces. In a specific embodiment, the KARI is derived from Schizosaccharomyces pombe or Schizosaccharomyces japonicus. In another specific embodiment, the KARI is encoded by SEQ ID NO: 57, SEQ ID NO: 59, or SEQ ID NO: 61.

Another aspect of the application relates to a recombinant microorganism comprising at least one nucleic acid molecule encoding a ketol-acid reductoisomerase (KARI), wherein said KARI is at least about 99% identical to SEQ ID NO: 64. In one embodiment, the KARI is derived from the genus Salmonella. In a specific embodiment, the KARI is derived from Salmonella enterica. In another specific embodiment, the KARI is encoded by SEQ ID NO: 63.

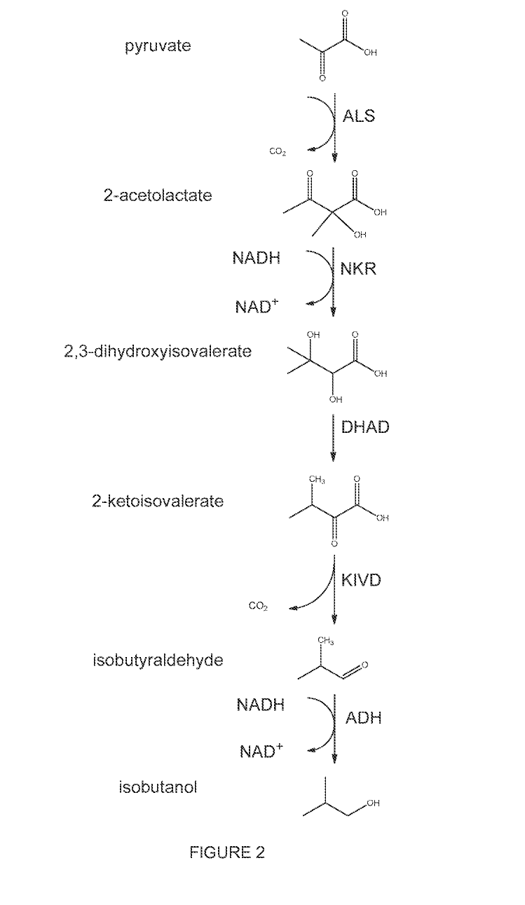

In an exemplary embodiment, pathway steps 2 and 5 of the isobutanol pathway may be carried out by KARI and ADH enzymes that utilize NADH (rather than NADPH) as a cofactor. It has been found previously that utilization of NADH-dependent KARI (NKR) and ADH enzymes to catalyze pathway steps 2 and 5, respectively, surprisingly enables production of isobutanol at theoretical yield and/or under anaerobic conditions. See, e.g., commonly owned and co-pending patent publication US 2010/0143997. An example of an NADH-dependent isobutanol pathway is illustrated in FIG. 2. Thus, in one embodiment, the recombinant microorganisms of the present invention may use an NKR to catalyze the conversion of acetolactate to produce 2,3-dihydroxyisovalerate. In another embodiment, the recombinant microorganisms of the present invention may use an NADH-dependent ADH to catalyze the conversion of isobutyraldehyde to produce isobutanol. In yet another embodiment, the recombinant microorganisms of the present invention may use both an NKR to catalyze the conversion of acetolactate to produce 2,3-dihydroxyisovalerate, and an NADH-dependent ADH to catalyze the conversion of isobutyraldehyde to produce isobutanol.

In an exemplary embodiment, the NKR is derived from a KARI that is at least about 80% identical to SEQ ID NO: 2. In another exemplary embodiment, the NKR is a KARI enzyme that has one or more modifications or mutations at positions corresponding to amino acids selected from: (a) alanine 71 of the Shewanella sp. KARI (SEQ ID NO: 2); (b) arginine 76 of the Shewanella sp. KARI (SEQ ID NO: 2); (c) serine 78 of the Shewanella sp. KARI; and (d) glutamine 110 of the Shewanella sp. KARI (SEQ ID NO: 2).

In one specific embodiment, the application is directed to KARI enzymes wherein the alanine corresponding to position 71 of the Shewanella sp. KARI (SEQ ID NO: 2) is replaced with an amino acid selected from serine, threonine, asparagine, or glutamine. In another specific embodiment, the application is directed to KARI enzymes wherein the arginine corresponding to position 76 of the Shewanella sp. KARI (SEQ ID NO: 2) is replaced with aspartic acid or glutamic acid. In yet another specific embodiment, the application is directed to KARI enzymes wherein the serine corresponding to position 78 of the Shewanella sp. KARI (SEQ ID NO: 2) is replaced with aspartic acid or glutamic acid. In yet another specific embodiment, the application is directed to KARI enzymes wherein the glutamine corresponding to position 110 of the Shewanella sp. KARI (SEQ ID NO: 2) is replaced with valine, alanine, isoleucine, leucine, methionine, phenylalanine, tryptophan, or tyrosine.

In another specific embodiment, the application relates to a KARI enzyme having four modifications or mutations at positions corresponding to amino acids selected from: (a) alanine 71 of the Shewanella sp. KARI (SEQ ID NO: 2); (b) arginine 76 of the Shewanella sp. KARI (SEQ ID NO: 2); (c) serine 78 of the Shewanella sp. KARI; and (d) glutamine 110 of the Shewanella sp. KARI (SEQ ID NO: 2).

In another exemplary embodiment, the NKR is derived from a KARI that is at least about 80% identical to SEQ ID NO: 10. In another exemplary embodiment, the NKR is a KARI enzyme that has one or more modifications or mutations at positions corresponding to amino acids selected from: (a) valine 48 of the L. lactis KARI (SEQ ID NO: 10); (b) arginine 49 of the L. lactis KARI (SEQ ID NO: 10); (c) lysine 52 of the L. lactis KARI (SEQ ID NO: 10); (d) serine 53 of the L. lactis KARI (SEQ ID NO: 10); (e) glutamic acid 59 of the L. lactis KARI (SEQ ID NO: 10): (f); leucine 85 of the L. lactis KARI (SEQ ID NO: 10); (g) isoleucine 89 of the L. lactis KARI (SEQ ID NO: 10); (h) lysine 118 of the L. lactis KARI (SEQ ID NO: 10); (i) threonine 182 of the L. lactis KARI (SEQ ID NO: 10); and (j) glutamic acid 320 of the L. lactis KARI (SEQ ID NO: 10).

In one specific embodiment, the application is directed to KARI enzymes wherein the valine corresponding to position 48 of the L. lactis KARI (SEQ ID NO: 2) is replaced with leucine or proline. In another specific embodiment, the application is directed to KARI enzymes wherein the arginine corresponding to position 49 of the L. lactis KARI (SEQ ID NO: 2) is replaced with valine, leucine, serine, or proline. In yet another specific embodiment, the application is directed to KARI enzymes wherein the lysine corresponding to position 52 of the L. lactis KARI (SEQ ID NO: 2) is replaced with leucine, alanine, isoleucine, methionine, phenylalanine, tryptophan, tyrosine, valine, aspartic acid, or glutamic acid. In yet another specific embodiment, the application is directed to KARI enzymes wherein the serine corresponding to position 53 of the L. lactis KARI (SEQ ID NO: 2) is replaced with aspartic acid or glutamic acid. In yet another specific embodiment, the application is directed to KARI enzymes wherein the glutamic acid corresponding to position 59 of the L. lactis KARI (SEQ ID NO: 2) is replaced with lysine, arginine, or histidine. In yet another specific embodiment, the application is directed to KARI enzymes wherein the leucine corresponding to position 85 of the L. lactis KARI (SEQ ID NO: 2) is replaced with threonine or alanine. In yet another specific embodiment, the application is directed to KARI enzymes wherein the isoleucine corresponding to position 89 of the L. lactis KARI (SEQ ID NO: 2) is replaced with alanine. In yet another specific embodiment, the application is directed to KARI enzymes wherein the lysine corresponding to position 118 of the L. lactis KARI (SEQ ID NO: 2) is replaced with glutamic acid or aspartic acid. In yet another specific embodiment, the application is directed to KARI enzymes wherein the threonine corresponding to position 182 of the L. lactis KARI (SEQ ID NO: 2) is replaced with serine, asparagine, or glutamine. In yet another specific embodiment, the application is directed to KARI enzymes wherein the glutamic acid corresponding to position 320 of the L. lactis KARI (SEQ ID NO: 2) is replaced with lysine, arginine, or histidine.

In another specific embodiment, the application relates to a KARI enzyme having seven modifications or mutations at positions corresponding to amino acids selected from: (a) valine 48 of the L. lactis KARI (SEQ ID NO: 10); (b) arginine 49 of the L. lactis KARI (SEQ ID NO: 10); (c) lysine 52 of the L. lactis KARI (SEQ ID NO: 10); (d) serine 53 of the L. lactis KARI (SEQ ID NO: 10): (e) glutamic acid 59 of the L. lactis KARI (SEQ ID NO: 10): (f) threonine 182 of the L. lactis KARI (SEQ ID NO: 10); and (g) glutamic acid 320 of the L. lactis KARI (SEQ ID NO: 10).

In yet another exemplary embodiment, the NKR is derived from a KARI that is at least about 99% identical to SEQ ID NO: 64. In another exemplary embodiment, the NKR is a KARI enzyme that has one or more modifications or mutations at positions corresponding to amino acids selected from: (a) alanine 71 of the S. enterica KARI (SEQ ID NO: 64); (b) arginine 76 of the S. enterica KARI (SEQ ID NO: 64); (c) serine 78 of the S. enterica KARI (SEQ ID NO: 64); (d) glutamine 110 of the S. enterica KARI (SEQ ID NO: 64); (e) aspartic acid 146 of the S. enterica KARI (SEQ ID NO: 64): (f) glycine 185 of the S. enterica KARI (SEQ ID NO: 64); and (g) lysine 433 of the S. enterica KARI (SEQ ID NO: 64).

In one specific embodiment, the application is directed to KARI enzymes wherein the alanine corresponding to position 71 of the S. enterica KARI (SEQ ID NO: 64) is replaced with an amino acid selected from serine, threonine, asparagine, or glutamine. In another specific embodiment, the application is directed to KARI enzymes wherein the arginine corresponding to position 76 of the S. enterica KARI (SEQ ID NO: 64) is replaced with aspartic acid or glutamic acid. In yet another specific embodiment, the application is directed to KARI enzymes wherein the serine corresponding to position 78 of the S. enterica KARI (SEQ ID NO: 64) is replaced with aspartic acid or glutamic acid. In yet another specific embodiment, the application is directed to KARI enzymes wherein the glutamine corresponding to position 110 of the S. enterica KARI (SEQ ID NO: 64) is replaced with valine, alanine, isoleucine, leucine, methionine, phenylalanine, tryptophan, or tyrosine. In yet another specific embodiment, the application is directed to KARI enzymes wherein the aspartic acid corresponding to position 146 of the S. enterica KARI (SEQ ID NO: 64) is replaced with glycine, cysteine, or proline. In yet another specific embodiment, the application is directed to KARI enzymes wherein the glycine corresponding to position 185 of the S. enterica KARI (SEQ ID NO: 64) is replaced with arginine, histidine, or lysine. In yet another specific embodiment, the application is directed to KARI enzymes wherein the lysine corresponding to position 433 of the S. enterica KARI (SEQ ID NO: 64) is replaced with glutamic acid or aspartic acid.

In another specific embodiment, the application relates to a KARI enzyme having seven modifications or mutations at positions corresponding to amino acids selected from: (a) alanine 71 of the S. enterica KARI (SEQ ID NO: 64); (b) arginine 76 of the S. enterica KARI (SEQ ID NO: 64); (c) serine 78 of the S. enterica KARI (SEQ ID NO: 64); (d) glutamine 110 of the S. enterica KARI (SEQ ID NO: 64); (e) aspartic acid 146 of the S. enterica KARI (SEQ ID NO: 64); (f) glycine 185 of the S. enterica KARI (SEQ ID NO: 64); and (g) lysine 433 of the S. enterica KARI (SEQ ID NO: 64).

Further included within the scope of the application are KARI enzymes, other than the Shewanella sp. KARI (SEQ ID NO: 2), the L. lactis KARI (SEQ ID NO: 10), or the S. enterica KARI (SEQ ID NO: 64) which contain modifications or mutations corresponding to those set out above. The nucleotide sequences for several KARI enzymes are known. A representative listing of KARI enzymes capable of being modified are disclosed in commonly owned and co-pending US Publication No. US 2010/0143997.

The corresponding positions of the KARI enzyme identified herein (e.g., the Shewanella sp. KARI, the L. lactis KARI, or the S. enterica KARI) may be readily identified for other KARI enzymes by one of skill in the art. Thus, given the defined region and the assays described in the present application, one with skill in the art can make one or a number of modifications which would result in an increased ability to utilize NADH, particularly for the conversion of acetolactate to 2,3-dihydroxyisovalerate, in any KARI enzyme of interest. Residues to be modified in accordance with the present application may include those described in Examples 4-5.

The application also includes fragments of the modified KARI enzymes which comprise at least 50, 100, 150, 200, 250, 300, 350, 400, 450, 500, 550, or 600 amino acid residues and retain one or more activities associated with KARI enzymes. Such fragments may be obtained by deletion mutation, by recombinant techniques that are routine and well-known in the art, or by enzymatic digestion of the KARI enzyme(s) of interest using any of a number of well-known proteolytic enzymes. The invention further includes nucleic acid molecules which encode the above described mutant KARI enzymes and KARI enzyme fragments.

Another aspect of the application relates to a recombinant microorganism comprising at least one nucleic acid molecule encoding a ketol-acid reductoisomerase (KARI), wherein said KARI has one or more modifications or mutations at positions corresponding to amino acids selected from: (a) alanine 71 of the Shewanella sp. KARI (SEQ ID NO: 2); (b) arginine 76 of the Shewanella sp. KARI (SEQ ID NO: 2); (c) serine 78 of the Shewanella sp. KARI; and (d) glutamine 110 of the Shewanella sp. KARI (SEQ ID NO: 2). Further included within the scope of the application are recombinant microorganisms comprising a KARI enzyme, other than the Shewanella sp. KARI (SEQ ID NO: 2), which contains modifications or mutations at positions corresponding to those set out above.

Yet another aspect of the application relates to a recombinant microorganism comprising at least one nucleic acid molecule encoding a ketol-acid reductoisomerase (KARI), wherein said KARI has one or more modifications or mutations at positions corresponding to amino acids selected from: (a) valine 48 of the L. lactis KARI (SEQ ID NO: 10); (b) arginine 49 of the L. lactis KARI (SEQ ID NO: 10); (c) lysine 52 of the L. lactis KARI (SEQ ID NO: 10); (d) serine 53 of the L. lactis KARI (SEQ ID NO: 10); (e) glutamic acid 59 of the L. lactis KARI (SEQ ID NO: 10): (f); leucine 85 of the L. lactis KARI (SEQ ID NO: 10); (g) isoleucine 89 of the L. lactis KARI (SEQ ID NO: 10); (h) lysine 118 of the L. lactis KARI (SEQ ID NO: 10); (i) threonine 182 of the L. lactis KARI (SEQ ID NO: 10); and (j) glutamic acid 320 of the L. lactis KARI (SEQ ID NO: 10). Further included within the scope of the application are recombinant microorganisms comprising a KARI enzyme, other than the L. lactis KARI (SEQ ID NO: 10), which contains modifications or mutations at positions corresponding to those set out above.

Another aspect of the application relates to a recombinant microorganism comprising at least one nucleic acid molecule encoding a ketol-acid reductoisomerase (KARI), wherein said KARI has one or more modifications or mutations at positions corresponding to amino acids selected from: (a) alanine 71 of the S. enterica KARI (SEQ ID NO: 64); (b) arginine 76 of the S. enterica KARI (SEQ ID NO: 64); (c) serine 78 of the S. enterica KARI (SEQ ID NO: 64); (d) glutamine 110 of the S. enterica KARI (SEQ ID NO: 64); (e) aspartic acid 146 of the S. enterica KARI (SEQ ID NO: 64); (f) glycine 185 of the S. enterica KARI (SEQ ID NO: 64); and (g) lysine 433 of the S. enterica KARI (SEQ ID NO: 64). Further included within the scope of the application are recombinant microorganisms comprising a KARI enzyme, other than the S. enterica KARI (SEQ ID NO: 64), which contains modifications or mutations at positions corresponding to those set out above.

Further within the scope of present application are recombinant microorganisms comprising at least one nucleic acid molecule encoding a ketol-acid reductoisomerase (KARI), wherein said KARI is at least about 80%, 85%, 90%, 91%, 92%, 93%, 94%, 95%, 96%, 97%, 98%, or 99% identical to SEQ ID NO: 2, SEQ ID NO: 4, SEQ ID NO: 6, SEQ ID NO: 8, SEQ ID NO: 10, SEQ ID NO; 28, SEQ ID NO: 40, or SEQ ID NO: 56. Also within the scope of present application are recombinant microorganisms comprising at least one nucleic acid molecule encoding a ketol-acid reductoisomerase (KARI), wherein said KARI is at least about 80%, 85%, 90%, 91%, 92%, 93%, 94%, 95%, 96%, 97%, 98%, or 99% identical to a KARI having one or more modifications or mutations at positions corresponding to amino acids selected from: (a) alanine 71 of the Shewanella sp. KARI (SEQ ID NO: 2); (b) arginine 76 of the Shewanella sp. KARI (SEQ ID NO: 2); (c) serine 78 of the Shewanella sp. KARI; and (d) glutamine 110 of the Shewanella sp. KARI (SEQ ID NO: 2). Also within the scope of present application are recombinant microorganisms comprising at least one nucleic acid molecule encoding a ketol-acid reductoisomerase (KARI), wherein said KARI is at least about 80%, 85%, 90%, 91%, 92%, 93%, 94%, 95%, 96%, 97%, 98%, or 99% identical to a KARI having one or more modifications or mutations at positions corresponding to amino acids selected from: (a) valine 48 of the L. lactis KARI (SEQ ID NO: 10); (b) arginine 49 of the L. lactis KARI (SEQ ID NO: 10); (c) lysine 52 of the L. lactis KARI (SEQ ID NO: 10); (d) serine 53 of the L. lactis KARI (SEQ ID NO: 10); (e) glutamic acid 59 of the L. lactis KARI (SEQ ID NO: 10): (f); leucine 85 of the L. lactis KARI (SEQ ID NO: 10); (g) isoleucine 89 of the L. lactis KARI (SEQ ID NO: 10); (h) lysine 118 of the L. lactis KARI (SEQ ID NO: 10); (i) threonine 182 of the L. lactis KARI (SEQ ID NO: 10); and (j) glutamic acid 320 of the L. lactis KARI (SEQ ID NO: 10).

Further within the scope of present application are recombinant microorganisms comprising at least one nucleic acid molecule encoding a ketol-acid reductoisomerase (KARI), wherein said KARI is at least about 99% identical to SEQ ID NO: 64. Also within the scope of present application are recombinant microorganisms comprising at least one nucleic acid molecule encoding a ketol-acid reductoisomerase (KARI), wherein said KARI is at least about 99% identical to a KARI having one or more modifications or mutations at positions corresponding to amino acids selected from: (a) alanine 71 of the S. enterica KARI (SEQ ID NO: 64); (b) arginine 76 of the S. enterica KARI (SEQ ID NO: 64); (c) serine 78 of the S. enterica KARI (SEQ ID NO: 64); (d) glutamine 110 of the S. enterica KARI (SEQ ID NO: 64); (e) aspartic acid 146 of the S. enterica KARI (SEQ ID NO: 64); (f) glycine 185 of the S. enterica KARI (SEQ ID NO: 64); and (g) lysine 433 of the S. enterica KARI (SEQ ID NO: 64).

In accordance with the invention, any number of mutations can be made to the KARI enzymes, and in a preferred aspect, multiple mutations can be made to result in an increased ability to utilize NADH for the conversion of acetolactate to 2,3-dihydroxyisovalerate. Such mutations include point mutations, frame shift mutations, deletions, and insertions, with one or more (e.g., one, two, three, four, five or more, etc.) point mutations preferred.

Mutations may be introduced into the KARI enzymes of the present application to create NKRs using any methodology known to those skilled in the art. Mutations may be introduced randomly by, for example, conducting a PCR reaction in the presence of manganese as a divalent metal ion cofactor. Alternatively, oligonucleotide directed mutagenesis may be used to create the NKRs which allows for all possible classes of base pair changes at any determined site along the encoding DNA molecule. In general, this technique involves annealing an oligonucleotide complementary (except for one or more mismatches) to a single stranded nucleotide sequence coding for the KARI enzyme of interest. The mismatched oligonucleotide is then extended by DNA polymerase, generating a double-stranded DNA molecule which contains the desired change in sequence in one strand. The changes in sequence can, for example, result in the deletion, substitution, or insertion of an amino acid. The double-stranded polynucleotide can then be inserted into an appropriate expression vector, and a mutant or modified polypeptide can thus be produced. The above-described oligonucleotide directed mutagenesis can, for example, be carried out via PCR.

In one aspect, the NADH-dependent activity of the modified or mutated KARI enzyme is increased.

In an exemplary embodiment, the catalytic efficiency of the modified or mutated KARI enzyme is improved for the cofactor NADH. Preferably, the catalytic efficiency of the modified or mutated KARI enzyme is improved by at least about 5% as compared to the wild-type or parental KARI for NADH. More preferably the catalytic efficiency of the modified or mutated KARI enzyme is improved by at least about 15% as compared to the wild-type or parental KARI for NADH. More preferably, the catalytic efficiency of the modified or mutated KARI enzyme is improved by at least about 25% as compared to the wild-type or parental KARI for NADH. More preferably, the catalytic efficiency of the modified or mutated KARI enzyme is improved by at least about 50% as compared to the wild-type or parental KARI for NADH. More preferably, the catalytic efficiency of the modified or mutated KARI enzyme is improved by at least about 75% as compared to the wild-type or parental KARI for NADH. More preferably, the catalytic efficiency of the modified or mutated KARI enzyme is improved by at least about 100% as compared to the wild-type or parental KARI for NADH. More preferably, the catalytic efficiency of the modified or mutated KARI enzyme is improved by at least about 300% as compared to the wild-type or parental KARI for NADH. More preferably, the catalytic efficiency of the modified or mutated KARI enzyme is improved by at least about 500% as compared to the wild-type or parental KARI for NADH. More preferably, the catalytic efficiency of the modified or mutated KARI enzyme is improved by at least about 1000% as compared to the wild-type or parental KARI for NADH. More preferably, the catalytic efficiency of the modified or mutated KARI enzyme is improved by at least about 5000% as compared to the wild-type or parental KARI for NADH.

In another exemplary embodiment, the catalytic efficiency of the modified or mutated KARI enzyme with NADH is increased with respect to the catalytic efficiency of the wild-type or parental enzyme with NADPH. Preferably, the catalytic efficiency of the modified or mutated KARI enzyme is at least about 10% of the catalytic efficiency of the wild-type or parental KARI enzyme for NADPH. More preferably, the catalytic efficiency of the modified or mutated KARI enzyme is at least about 25% of the catalytic efficiency of the wild-type or parental KARI enzyme for NADPH. More preferably, the catalytic efficiency of the modified or mutated KARI enzyme is at least about 50% of the catalytic efficiency of the wild-type or parental KARI enzyme for NADPH. More preferably, the catalytic efficiency of the modified or mutated KARI enzyme is at least about 75%, 85%, 95% of the catalytic efficiency of the wild-type or parental KARI enzyme for NADPH.

In another exemplary embodiment, the K_{M} of the KARI enzyme for NADH is decreased relative to the wild-type or parental enzyme. A change in K_{M} is evidenced by at least a 5% or greater increase or decrease in K_{M} compared to the wild-type KARI enzyme. In certain embodiments, modified or mutated KARI enzymes of the present invention may show greater than 10 times decreased K_{M} for NADH compared to the wild-type or parental KARI enzyme. In certain embodiments, modified or mutated KARI enzymes of the present invention may show greater than 30 times decreased K_{M} for NADH compared to the wild-type or parental KARI enzyme.

In another exemplary embodiment, the k_{cat} of the KARI enzyme with NADH is increased relative to the wild-type or parental enzyme. A change in k_{cat} is evidenced by at least a 5% or greater increase or decrease in K_{M} compared to the wild-type KARI enzyme. In certain embodiments, modified or mutated KARI enzymes of the present invention may show greater than 50% increased k_{cat} for NADH compared to the wild-type or parental KARI enzyme. In certain embodiments, modified or mutated KARI enzymes of the present invention may show greater than 100% increased k_{cat} for NADH compared to the wild-type or parental KARI enzyme. In certain embodiments, modified or mutated KARI enzymes of the present invention may show greater than 200% increased k_{cat} for NADH compared to the wild-type or parental KARI enzyme.

Recombinant Microorganisms Comprising KARI with Improved Properties

In addition to isobutanol producing metabolic pathways, a number of biosynthetic pathways use KARI enzymes to catalyze a reaction step, including pathways for the production of isoleucine, leucine, valine, pantothenate, coenzyme A, 1-butanol, 2-methyl-1-butanol, 3-methyl-1-butanol, 3-methyl-1-pentanol, 4-methyl-1-pentanol, 4-methyl-1-hexanol, and 5-methyl-1-heptanol. A representative list of the engineered biosynthetic pathways utilizing KARI enzymes is provided in Table 1.

TABLE 1
Biosynthetic Pathways Utilizing
KARI to Catalyze a Reaction Step.

| Biosynthetic | |
|---|---|
| Pathway | Reference^{a} |
| Isobutanol | US 2009/0226991 (Feldman et al.), US 2011/0020889 |
| | (Feldman et al.), and US 2010/0143997 (Buelter et al.) |
| Leucine | WO/2001/021772 (Yocum et al.) and McCourt et al., |
| | 2006, Amino Acids 31: 173-210 |
| Valine | WO/2001/021772 (Yocum et al.) and McCourt et al., |
| | 2006, Amino Acids 31: 173-210 |
| Pantothenic Acid | WO/2001/021772 (Yocum et al.) |
| Coenzyme A | WO/2001/021772 (Yocum et al.) |
| 1-Butanol | WO/2010/017230 (Lynch), WO/2010/031772 |
| | (Wu at al.), and KR2011002130 (Lee et al.) |
| 2-Methyl-1- | WO/2008/098227 (Liao et al.), WO/2009/076480 |
| Butanol | (Picataggio et al.), and Atsumi et al., 2008, |
| | Nature 451: 86-89 |
| 3-Methyl-1- | WO/2008/098227 (Liao et al.), Atsumi et al., 2008, |
| Butanol | Nature 451: 86-89, and Connor et al., 2008, |
| | Appl. Environ. Microbiol. 74: 5769-5775 |
| 3-Methyl-1- | WO/2010/045629 (Liao et al.), Zhang at al., 2008, |
| Pentanol | Proc Natl Acad Sci USA 105: 20653-20658 |
| 4-Methyl-1- | WO/2010/045629 (Liao et al.), Zhang et al., 2008, |
| Pentanol | Proc Natl Acad Sci USA 105: 20653-20658 |
| 4-Methyl-1- | WO/2010/045629 (Liao et al.), Zhang et al., 2008, |
| Hexanol | Proc Natl Acad Sci USA 105:20653-20658 |
| 5-Methyl-1- | WO/2010/045629 (Liao et al.), Zhang et al., 2008, |
| Heptanol | Proc Natl Acad Sci USA 105: 20653-20658 |

^{a}The contents of each of the references in this table are herein incorporated by reference in their entireties for all purposes.

As described above, each of these biosynthetic pathways uses a KARI enzyme to catalyze a reaction step. Therefore, the product yield from these biosynthetic pathways will in part depend upon the activity of KARI.

As will be understood by one skilled in the art equipped with the present disclosure, the KARI enzymes described herein would have utility in any of the above-described pathways. Thus, in an additional aspect, the present application relates to a recombinant microorganism comprising a KARI-requiring biosynthetic pathway, wherein said recombinant microorganism comprises at least one nucleic acid molecule encoding a KARI that is at least about 80% identical to SEQ ID NO: 2, SEQ ID NO: 4, SEQ ID NO: 6, SEQ ID NO: 8, SEQ ID NO: 10, SEQ ID NO: 28, SEQ ID NO: 40, or SEQ ID NO: 58. In one embodiment, the KARI is derived from the genus Shewanella. In a specific embodiment, the KARI is derived from Shewanella sp. strain MR-4. In another specific embodiment, the isolated nucleic acid molecule is comprised of SEQ ID NO: 1. In another embodiment, the KARI is derived from the genus Vibrio. In a specific embodiment, the KARI is derived from Vibrio fischei. In another specific embodiment, the isolated nucleic acid molecule is comprised of SEQ ID NO: 3. In yet another embodiment, the KARI is derived from the genus Gramella. In a specific embodiment, the KARI is derived from Gramella forsetii. In another specific embodiment, the isolated nucleic acid molecule is comprised of SEQ ID NO: 5. In yet another embodiment, the KARI is derived from the genus Cytophaga. In a specific embodiment, the KARI is derived from Cytophaga hutchinsonii. In another specific embodiment, the isolated nucleic acid molecule is comprised of SEQ ID NO: 7. In yet another embodiment, the KARI is derived from a genus selected from Lactococcus and Streptococcus. In a specific embodiment, the KARI is derived from Lactococcus lactis, Streptococcus equinus, or Streptococcus infantarius. In another specific embodiment, the KARI is encoded by SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25. In yet another embodiment, the KARI is derived from the genus Methanococcus. In a specific embodiment, the KARI is derived from Methanococcus maripaludis, Methanococcus vannielii, or Methanococcus voltae. In another specific embodiment, the KARI is encoded by SEQ ID NO: 27, SEQ ID NO: 29, SEQ ID NO: 31, SEQ ID NO: 33, SEQ ID NO: 35, or SEQ ID NO: 37. In yet another embodiment, the KARI is derived from a genus selected from Zymomonas, Erythrobacter, Sphingomonas, Sphingobium, and Novosphingobium. In a specific embodiment, the KARI is derived from Zymomonas mobilis, Erythrobacter litoralis, Sphingomonas wittichii, Sphingobium japonicum, Sphingobium chlorophenolicum, or Novosphingobium nitrogenifigens. In another specific embodiment, the KARI is encoded by SEQ ID NO: 39, SEQ ID NO: 41, SEQ ID NO: 43, SEQ ID NO: 45, SEQ ID NO: 47, SEQ ID NO: 49, SEQ ID NO: 51, or SEQ ID NO: 53. In yet another embodiment, the KARI is derived from the genus Bacteroides. In a specific embodiment, the KARI is derived from Bacteroides thetaiotaomicron. In another specific embodiment, the KARI is encoded by SEQ ID NO: 55. In yet another embodiment, the KARI is derived from the genus Schizosaccharomyces. In a specific embodiment, the KARI is derived from Schizosaccharomyces pombe or Schizosaccharomyces japonicus. In another specific embodiment, the KARI is encoded by SEQ ID NO: 57, SEQ ID NO: 59, or SEQ ID NO: 61. In yet another embodiment, the KARI has one or more modifications or mutations at positions corresponding to amino acids selected from: (a) alanine 71 of the Shewanella sp. KARI (SEQ ID NO: 2); (b) arginine 76 of the Shewanella sp. KARI (SEQ ID NO: 2); (c) serine 78 of the Shewanella sp. KARI; and (d) glutamine 110 of the Shewanella sp. KARI (SEQ ID NO: 2). In yet another embodiment, the KARI has one or more modifications or mutations at positions corresponding to amino acids selected from: (a) valine 48 of the L. lactis KARI (SEQ ID NO: 10): (b) arginine 49 of the L. lactis KARI (SEQ ID NO: 10); (c) lysine 52 of the L. lactis KARI (SEQ ID NO: 10); (d) serine 53 of the L. lactis KARI (SEQ ID NO: 10); (e) glutamic acid 59 of the L. lactis KARI (SEQ ID NO: 10): (f); leucine 85 of the L. lactis KARI (SEQ ID NO: 10); (g) isoleucine 89 of the L. lactis KARI (SEQ ID NO: 10); (h) lysine 118 of the L. lactis KARI (SEQ ID NO: 10); (i) threonine 182 of the L. lactis KARI (SEQ ID NO: 10); and (j) glutamic acid 320 of the L. lactis KARI (SEQ ID NO: 10).

In an additional aspect, the present application relates to a recombinant microorganism comprising a KARI-requiring biosynthetic pathway, wherein said recombinant microorganism comprises at least one nucleic acid molecule encoding a KARI that is at least about 99% identical to SEQ ID NO: 64. In one embodiment, the KARI is derived from the genus Salmonella. In a specific embodiment, the KARI is derived from Salmonella enterica. In another specific embodiment, the KARI is encoded by SEQ ID NO: 63. In yet another embodiment, the KARI has one or more modifications or mutations at positions corresponding to amino acids selected from: (a) alanine 71 of the S. enterica KARI (SEQ ID NO: 64); (b) arginine 76 of the S. enterica KARI (SEQ ID NO: 64); (c) serine 78 of the S. enterica KARI (SEQ ID NO: 64); (d) glutamine 110 of the S. enterica KARI (SEQ ID NO: 64); (e) aspartic acid 146 of the S. enterica KARI (SEQ ID NO: 64); (f) glycine 185 of the S. enterica KARI (SEQ ID NO: 64); and (g) lysine 433 of the S. enterica KARI (SEQ ID NO: 64).

As used herein, a “KARI-requiring biosynthetic pathway” refers to any metabolic pathway which utilizes KARI to convert acetolactate to 2,3-dihydroxyisovalerate or 2-aceto-2-hydroxy-butanoate to 2,3-dihydroxy-3-methylvalerate. Examples of KARI-requiring biosynthetic pathways include, but are not limited to, isobutanol, isoleucine, leucine, valine, pantothenate, coenzyme A, 1-butanol, 2-methyl-1-butanol, 3-methyl-1-butanol, 3-methyl-1-pentanol, 4-methyl-1-pentanol, 4-methyl-1-hexanol, and 5-methyl-1-heptanol metabolic pathways. The metabolic pathway may naturally occur in a microorganism (e.g., a natural pathway for the production of valine) or arise from the introduction of one or more heterologous polynucleotides through genetic engineering. In an exemplary embodiment, the recombinant microorganisms expressing the KARI-requiring biosynthetic pathway are yeast cells.

### The Microorganism in General

As described herein, the recombinant microorganisms of the present invention can express a plurality of heterologous and/or native enzymes involved in pathways for the production of a beneficial metabolite such as isobutanol.

As described herein, “engineered” or “modified” microorganisms are produced via the introduction of genetic material into a host or parental microorganism of choice and/or by modification of the expression of native genes, thereby modifying or altering the cellular physiology and biochemistry of the microorganism. Through the introduction of genetic material and/or the modification of the expression of native genes the parental microorganism acquires new properties, e.g., the ability to produce a new, or greater quantities of, an intracellular and/or extracellular metabolite. As described herein, the introduction of genetic material into and/or the modification of the expression of native genes in a parental microorganism results in a new or modified ability to produce beneficial metabolites such as isobutanol from a suitable carbon source. The genetic material introduced into and/or the genes modified for expression in the parental microorganism contains gene(s), or parts of genes, coding for one or more of the enzymes involved in a biosynthetic pathway for the production of isobutanol and may also include additional elements for the expression and/or regulation of expression of these genes, e.g., promoter sequences.

In addition to the introduction of a genetic material into a host or parental microorganism, an engineered or modified microorganism can also include the alteration, disruption, deletion or knocking-out of a gene or polynucleotide to alter the cellular physiology and biochemistry of the microorganism. Through the alteration, disruption, deletion or knocking-out of a gene or polynucleotide, the microorganism acquires new or improved properties (e.g., the ability to produce a new metabolite or greater quantities of an intracellular metabolite, to improve the flux of a metabolite down a desired pathway, and/or to reduce the production of by-products).

Recombinant microorganisms provided herein may also produce metabolites in quantities not available in the parental microorganism. A “metabolite” refers to any substance produced by metabolism or a substance necessary for or taking part in a particular metabolic process. A metabolite can be an organic compound that is a starting material (e.g., glucose or pyruvate), an intermediate (e.g., 2-ketoisovalerate), or an end product (e.g., isobutanol) of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

The disclosure identifies specific genes useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutations and silent mutations. Such modified or mutated polynucleotides and polypeptides can be screened for expression of a functional enzyme using methods known in the art.

Due to the inherent degeneracy of the genetic code, other polynucleotides which encode substantially the same or functionally equivalent polypeptides can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17: 477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24: 216-8). Methodology for optimizing a nucleotide sequence for expression in a plant is provided, for example, in U.S. Pat. No. 6,015,891, and the references cited therein.

Those of skill in the art will recognize that, due to the degenerate nature of the genetic code, a variety of DNA compounds differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for generating metabolites are encompassed by the microorganisms and methods provided herein.

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., 1994, Methods in Mol Biol 25: 365-89).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See commonly owned and co-pending application US 2009/0226991. A typical algorithm used comparing a molecule sequence to a database containing a large number of sequences from different organisms is the computer program BLAST. When searching a database containing sequences from a large number of different organisms, it is typical to compare amino acid sequences. Database searching using amino acid sequences can be measured by algorithms described in commonly owned U.S. Pat. No. 8,017,375.

It is understood that a range of microorganisms can be modified to include an isobutanol producing metabolic pathway suitable for the production of isobutanol. In various embodiments, the microorganisms may be selected from yeast microorganisms. Yeast microorganisms for the production of isobutanol may be selected based on certain characteristics:

One characteristic may include the property that the microorganism is selected to convert various carbon sources into isobutanol. The term “carbon source” generally refers to a substance suitable to be used as a source of carbon for prokaryotic or eukaryotic cell growth. Examples of suitable carbon sources are described in commonly owned U.S. Pat. No. 8,017,375. Accordingly, in one embodiment, the recombinant microorganism herein disclosed can convert a variety of carbon sources to products, including but not limited to glucose, galactose, mannose, xylose, arabinose, lactose, sucrose, CO₂, and mixtures thereof.

The recombinant microorganism may thus further include a pathway for the production of isobutanol from five-carbon (pentose) sugars including xylose. Most yeast species metabolize xylose via a complex route, in which xylose is first reduced to xylitol via a xylose reductase (XR) enzyme. The xylitol is then oxidized to xylulose via a xylitol dehydrogenase (XDH) enzyme. The xylulose is then phosphorylated via a xylulokinase (XK) enzyme. This pathway operates inefficiently in yeast species because it introduces a redox imbalance in the cell. The xylose-to-xylitol step uses primarily NADPH as a cofactor (generating NADP+), whereas the xylitol-to-xylulose step uses NAD+ as a cofactor (generating NADH). Other processes must operate to restore the redox imbalance within the cell. This often means that the organism cannot grow anaerobically on xylose or other pentose sugars. Accordingly, a yeast species that can efficiently ferment xylose and other pentose sugars into a desired fermentation product is therefore very desirable.

Thus, in one aspect, the recombinant microorganism is engineered to express a functional exogenous xylose isomerase. Exogenous xylose isomerases (XI) functional in yeast are known in the art. See, e.g., Rajgarhia et al., U.S. Pat. No. 7,943,366, which is herein incorporated by reference in its entirety. In an embodiment according to this aspect, the exogenous XI gene is operatively linked to promoter and terminator sequences that are functional in the yeast cell. In a preferred embodiment, the recombinant microorganism further has a deletion or disruption of a native gene that encodes for an enzyme (e.g., XR and/or XDH) that catalyzes the conversion of xylose to xylitol. In a further preferred embodiment, the recombinant microorganism also contains a functional, exogenous xylulokinase (XK) gene operatively linked to promoter and terminator sequences that are functional in the yeast cell. In one embodiment, the xylulokinase (XK) gene is overexpressed.

In one embodiment, the yeast microorganism has reduced or no pyruvate decarboxylase (PDC) activity. PDC catalyzes the decarboxylation of pyruvate to acetaldehyde, which is then reduced to ethanol by ADH via an oxidation of NADH to NAD+. Ethanol production is the main pathway to oxidize the NADH from glycolysis. Deletion, disruption, or mutation of this pathway increases the pyruvate and the reducing equivalents (NADH) available for a biosynthetic pathway which uses pyruvate as the starting material and/or as an intermediate. Accordingly, deletion, disruption, or mutation of one or more genes encoding for pyruvate decarboxylase and/or a positive transcriptional regulator thereof can further increase the yield of the desired pyruvate-derived metabolite (e.g., isobutanol). In one embodiment, said pyruvate decarboxylase gene targeted for disruption, deletion, or mutation is selected from the group consisting of PDC1, PDC5, and PDC6, or homologs or variants thereof. In another embodiment, all three of PDC1, PDC5, and PDC6 are targeted for disruption, deletion, or mutation. In yet another embodiment, a positive transcriptional regulator of the PDC1, PDC5, and/or PDC6 is targeted for disruption, deletion or mutation. In one embodiment, said positive transcriptional regulator is PDC2, or homologs or variants thereof.

As is understood by those skilled in the art, there are several additional mechanisms available for reducing or disrupting the activity of a protein encoded by PDC1, PDC5, PDC6, and/or PDC2, including, but not limited to, the use of a regulated promoter, use of a weak constitutive promoter, disruption of one of the two copies of the gene in a diploid yeast, disruption of both copies of the gene in a diploid yeast, expression of an anti-sense nucleic acid, expression of an siRNA, over expression of a negative regulator of the endogenous promoter, alteration of the activity of an endogenous or heterologous gene, use of a heterologous gene with lower specific activity, the like or combinations thereof. Yeast strains with reduced PDC activity are described in commonly owned U.S. Pat. No. 8,017,375, as well as commonly owned and co-pending US Patent Publication No. 2011/0183392.

In another embodiment, the microorganism has reduced glycerol-3-phosphate dehydrogenase (GPD) activity. GPD catalyzes the reduction of dihydroxyacetone phosphate (DHAP) to glycerol-3-phosphate (G3P) via the oxidation of NADH to NAD+. Glycerol is then produced from G3P by Glycerol-3-phosphatase (GPP). Glycerol production is a secondary pathway to oxidize excess NADH from glycolysis. Reduction or elimination of this pathway would increase the pyruvate and reducing equivalents (NADH) available for the production of a pyruvate-derived metabolite (e.g., isobutanol). Thus, disruption, deletion, or mutation of the genes encoding for glycerol-3-phosphate dehydrogenases can further increase the yield of the desired metabolite (e.g., isobutanol). Yeast strains with reduced GPD activity are described in commonly owned and co-pending US Patent Publication Nos. 2011/0020889 and 2011/0183392.

In yet another embodiment, the microorganism has reduced 3-keto acid reductase (3-KAR) activity. 3-KARs catalyze the conversion of 3-keto acids (e.g., acetolactate) to 3-hydroxyacids (e.g., DH2 MB). Yeast strains with reduced 3-KAR activity are described in commonly owned U.S. Pat. Nos. 8,133,715, 8,153,415, and 8,158,404, which are herein incorporated by reference in their entireties.

In yet another embodiment, the microorganism has reduced aldehyde dehydrogenase (ALDH) activity. Aldehyde dehydrogenases catalyze the conversion of aldehydes (e.g., isobutyraldehyde) to acid by-products (e.g., isobutyrate). Yeast strains with reduced ALDH activity are described in commonly owned U.S. Pat. Nos. 8,133,715, 8,153,415, and 8,158,404, which are herein incorporated by reference in their entireties.

In one embodiment, the yeast microorganisms may be selected from the “Saccharomyces Yeast Clade”, as described in commonly owned U.S. Pat. No. 8,017,375.

The term “Saccharomyces sensu stricto” taxonomy group is a cluster of yeast species that are highly related to S. cerevisiae (Rainieri et al., 2003, J. Biosci Bioengin 96: 1-9). Saccharomyces sensu stricto yeast species include but are not limited to S. cerevisiae, S. kudriavzevii, S. mikatae, S. bayanus, S. uvarum, S. carocanis and hybrids derived from these species (Masneuf et al., 1998, Yeast 7: 61-72).

An ancient whole genome duplication (WGD) event occurred during the evolution of the hemiascomycete yeast and was discovered using comparative genomic tools (Kellis et al., 2004, Nature 428: 617-24; Dujon et al., 2004, Nature 430:35-44; Langkjaer et al., 2003, Nature 428: 848-52; Wolfe et al., 1997, Nature 387: 708-13). Using this major evolutionary event, yeast can be divided into species that diverged from a common ancestor following the WGD event (termed “post-WGD yeast” herein) and species that diverged from the yeast lineage prior to the WGD event (termed “pre-WGD yeast” herein).

Accordingly, in one embodiment, the yeast microorganism may be selected from a post-WGD yeast genus, including but not limited to Saccharomyces and Candida. The favored post-WGD yeast species include: S. cerevisiae, S. uvarum, S. bayanus, S. paradoxus, S. castelli, and C. glabrata.

In another embodiment, the yeast microorganism may be selected from a pre-whole genome duplication (pre-WGD) yeast genus including but not limited to Saccharomyces, Kluyveromyces, Candida, Pichia, Issatchenkia, Debaryomyces, Hansenula, Yarrowia and, Schizosaccharomyces. Representative pre-WGD yeast species include: S. kluyveri, K. thermotolerans, K. marxianus, K. waltii, K. lactis, C. tropicalis, P. pastoris, P. anomala, P. stipitis, I. orientalis, I. occidentalis, I. scutulata, D. hansenii, H. anomala, Y. lipolytica, and S. pombe.

A yeast microorganism may be either Crabtree-negative or Crabtree-positive as described in described in commonly owned U.S. Pat. No. 8,017,375. In one embodiment the yeast microorganism may be selected from yeast with a Crabtree-negative phenotype including but not limited to the following genera: Saccharomyces, Kluyveromyces, Pichia, Issatchenkia, Hansenula, and Candida. Crabtree-negative species include but are not limited to: S. kluyvenri, K. lactis, K. marxianus, P. anomala, P. stipitis, I. orientalis, I. occidentalis, I. scutulata, H. anomala, and C. utilis. In another embodiment, the yeast microorganism may be selected from yeast with a Crabtree-positive phenotype, including but not limited to Saccharomyces, Kluyveromyces, Zygosaccharomyces, Debaryomyces, Pichia and Schizosaccharomyces. Crabtree-positive yeast species include but are not limited to: S. cerevisiae, S. uvarum, S. bayanus, S. paradoxus, S. castelli, K. thermotolerans, C. glabrata, Z. bailli, Z. rouxii, D. hansenii, P. pastorius, and S. pombe.

Another characteristic may include the property that the microorganism is that it is non-fermenting. In other words, it cannot metabolize a carbon source anaerobically while the yeast is able to metabolize a carbon source in the presence of oxygen. Nonfermenting yeast refers to both naturally occurring yeasts as well as genetically modified yeast. During anaerobic fermentation with fermentative yeast, the main pathway to oxidize the NADH from glycolysis is through the production of ethanol. Ethanol is produced by alcohol dehydrogenase (ADH) via the reduction of acetaldehyde, which is generated from pyruvate by pyruvate decarboxylase (PDC). In one embodiment, a fermentative yeast can be engineered to be non-fermentative by the reduction or elimination of the native PDC activity. Thus, most of the pyruvate produced by glycolysis is not consumed by PDC and is available for the isobutanol pathway. Deletion of this pathway increases the pyruvate and the reducing equivalents available for the biosynthetic pathway. Fermentative pathways contribute to low yield and low productivity of pyruvate-derived metabolites such as isobutanol. Accordingly, deletion of one or more PDC genes may increase yield and productivity of a desired metabolite (e.g., isobutanol).

In some embodiments, the recombinant microorganisms may be microorganisms that are non-fermenting yeast microorganisms, including, but not limited to those, classified into a genera selected from the group consisting of Tricosporon, Rhodotorula, Myxozyma, or Candida. In a specific embodiment, the non-fermenting yeast is C. xestobii.

### Methods in General

### Identification of KARI Homologs

Any method can be used to identify genes that encode for enzymes that are homologous to the genes described herein (e.g., KARI homologs). Generally, genes that are homologous or similar to the KARIs described herein may be identified by functional, structural, and/or genetic analysis. In most cases, homologous or similar genes and/or homologous or similar enzymes will have functional, structural, or genetic similarities.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous genes, proteins, or enzymes, techniques may include, but not limited to, cloning a gene by PCR using primers based on a published sequence of a gene/enzyme or by degenerate PCR using degenerate primers designed to amplify a conserved region among ketol-acid reductoisomerase genes. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K. Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, or MetaCYC. The candidate gene or enzyme may be identified within the above mentioned databases in accordance with the teachings herein.

### Genetic Insertions and Deletions

Any method can be used to introduce a nucleic acid molecule into yeast and many such methods are well known. For example, transformation and electroporation are common methods for introducing nucleic acid into yeast cells. See, e.g., Gietz et al., 1992, Nuc Acids Res. 27: 69-74; Ito et al., 1983, J. Bacteriol. 153: 163-8; and Becker et al., 1991, Methods in Enzymology 194: 182-7.

In an embodiment, the integration of a gene of interest into a DNA fragment or target gene of a yeast microorganism occurs according to the principle of homologous recombination. According to this embodiment, an integration cassette containing a module comprising at least one yeast marker gene and/or the gene to be integrated (internal module) is flanked on either side by DNA fragments homologous to those of the ends of the targeted integration site (recombinogenic sequences). After transforming the yeast with the cassette by appropriate methods, a homologous recombination between the recombinogenic sequences may result in the internal module replacing the chromosomal region in between the two sites of the genome corresponding to the recombinogenic sequences of the integration cassette. (Orr-Weaver et al., 1981, PNAS USA 78: 6354-58).

In an embodiment, the integration cassette for integration of a gene of interest into a yeast microorganism includes the heterologous gene under the control of an appropriate promoter and terminator together with the selectable marker flanked by recombinogenic sequences for integration of a heterologous gene into the yeast chromosome. In an embodiment, the heterologous gene includes an appropriate native gene desired to increase the copy number of a native gene(s). The selectable marker gene can be any marker gene used in yeast, including but not limited to, HIS3, TRP1, LEU2, URA3, bar, ble, hph, and kan. The recombinogenic sequences can be chosen at will, depending on the desired integration site suitable for the desired application.

In another embodiment, integration of a gene into the chromosome of the yeast microorganism may occur via random integration (Kooistra et al., 2004, Yeast 21: 781-792).

Additionally, in an embodiment, certain introduced marker genes are removed from the genome using techniques well known to those skilled in the art. For example, URA3 marker loss can be obtained by plating URA3 containing cells in FOA (5-fluoro-orotic acid) containing medium and selecting for FOA resistant colonies (Boeke et al., 1984, Mol. Gen. Genet 197: 345-47).

The exogenous nucleic acid molecule contained within a yeast cell of the disclosure can be maintained within that cell in any form. For example, exogenous nucleic acid molecules can be integrated into the genome of the cell or maintained in an episomal state that can stably be passed on (“inherited”) to daughter cells. Such extra-chromosomal genetic elements (such as plasmids, mitochondrial genome, etc.) can additionally contain selection markers that ensure the presence of such genetic elements in daughter cells. Moreover, the yeast cells can be stably or transiently transformed. In addition, the yeast cells described herein can contain a single copy, or multiple copies of a particular exogenous nucleic acid molecule as described above.

### Reduction of Enzymatic Activity

Yeast microorganisms within the scope of the invention may have reduced enzymatic activity such as reduced PDC, GPD, ALDH, or 3-KAR activity. The term “reduced” as used herein with respect to a particular polypeptide activity refers to a lower level of polypeptide activity than that measured in a comparable yeast cell of the same species. The term reduced also refers to the elimination of polypeptide activity as compared to a comparable yeast cell of the same species. Thus, yeast cells lacking activity for an endogenous PDC, GPD, ALDH, or 3-KAR are considered to have reduced activity for PDC, GPD. ALDH, or 3-KAR since most, if not all, comparable yeast strains have at least some activity for PDC, GPD, ALDH, or 3-KAR. Such reduced PDC, GPD, ALDH, or 3-KAR activities can be the result of lower PDC, GPD, ALDH, or 3-KAR concentration (e.g., via reduced expression), lower specific activity of the PDC. GPD, ALDH, or 3-KAR, or a combination thereof. Many different methods can be used to make yeast having reduced PDC, GPD, ALDH, or 3-KAR activity. For example, a yeast cell can be engineered to have a disrupted PDC-, GPD-, ALDH-, or 3-KAR-encoding locus using common mutagenesis or knock-out technology. See, e.g., Methods in Yeast Genetics (1997 edition), Adams, Gottschling, Kaiser, and Stems, Cold Spring Harbor Press (1998). In addition, a yeast cell can be engineered to partially or completely remove the coding sequence for a particular PDC, GPD, ALDH, or 3-KAR. Furthermore, the promoter sequence and/or associated regulatory elements can be mutated, disrupted, or deleted to reduce the expression of a PDC, GPD, ALDH, or 3-KAR. Moreover, certain point-mutation(s) can be introduced which results in a PDC, GPD, ALDH, or 3-KAR with reduced activity. Also included within the scope of this invention are yeast strains which when found in nature, are substantially free of one or more PDC, GPD, ALDH, or 3-KAR activities.

Alternatively, antisense technology can be used to reduce PDC, GPD, ALDH, or 3-KAR activity. For example, yeasts can be engineered to contain a cDNA that encodes an antisense molecule that prevents a PDC, GPD, ALDH, or 3-KAR from being made. The term “antisense molecule” as used herein encompasses any nucleic acid molecule that contains sequences that correspond to the coding strand of an endogenous polypeptide. An antisense molecule also can have flanking sequences (e.g., regulatory sequences). Thus antisense molecules can be ribozymes or antisense oligonucleotides. A ribozyme can have any general structure including, without limitation, hairpin, hammerhead, or axhead structures, provided the molecule cleaves RNA.

### Overexpression of Heterologous Genes

Methods for overexpressing a polypeptide from a native or heterologous nucleic acid molecule are well known. Such methods include, without limitation, constructing a nucleic acid sequence such that a regulatory element promotes the expression of a nucleic acid sequence that encodes the desired polypeptide. Typically, regulatory elements are DNA sequences that regulate the expression of other DNA sequences at the level of transcription. Thus, regulatory elements include, without limitation, promoters, enhancers, and the like. For example, the exogenous genes can be under the control of an inducible promoter or a constitutive promoter. Moreover, methods for expressing a polypeptide from an exogenous nucleic acid molecule in yeast are well known. For example, nucleic acid constructs that are used for the expression of exogenous polypeptides within Kluyveromyces and Saccharomyces are well known (see, e.g., U.S. Pat. Nos. 4,859,596 and 4,943,529, for Kluyveromyces and, e.g., Gellissen et al., Gene 190(1):87-97 (1997) for Saccharomyces). Yeast plasmids have a selectable marker and an origin of replication. In addition certain plasmids may also contain a centromeric sequence. These centromeric plasmids are generally a single or low copy plasmid. Plasmids without a centromeric sequence and utilizing either a 2 micron (S. cerevisiae) or 1.6 micron (K. lactis) replication origin are high copy plasmids. The selectable marker can be either prototrophic, such as HIS3. TRP1, LEU2, URA3 or ADE2, or antibiotic resistance, such as, bar, ble, hph, or kan.

In another embodiment, heterologous control elements can be used to activate or repress expression of endogenous genes. Additionally, when expression is to be repressed or eliminated, the gene for the relevant enzyme, protein or RNA can be eliminated by known deletion techniques.

As described herein, any yeast within the scope of the disclosure can be identified by selection techniques specific to the particular polypeptide (e.g. an isobutanol pathway enzyme) being expressed, over-expressed or repressed. Methods of identifying the strains with the desired phenotype are well known to those skilled in the art. Such methods include, without limitation, PCR, RT-PCR, and nucleic acid hybridization techniques such as Northern and Southern analysis, altered growth capabilities on a particular substrate or in the presence of a particular substrate, a chemical compound, a selection agent and the like. In some cases, immunohistochemistry and biochemical techniques can be used to determine if a cell contains a particular nucleic acid by detecting the expression of the encoded polypeptide. For example, an antibody having specificity for an encoded enzyme can be used to determine whether or not a particular yeast cell contains that encoded enzyme. Further, biochemical techniques can be used to determine if a cell contains a particular nucleic acid molecule encoding an enzymatic polypeptide by detecting a product produced as a result of the expression of the enzymatic polypeptide. For example, transforming a cell with a vector encoding acetolactate synthase and detecting increased acetolactate concentrations compared to a cell without the vector indicates that the vector is both present and that the gene product is active. Methods for detecting specific enzymatic activities or the presence of particular products are well known to those skilled in the art. For example, the presence of acetolactate can be determined as described by Hugenholtz and Starrenburg, 1992, Appl. Micro. Biot. 38:17-22.

### Increase of Enzymatic Activity

Yeast microorganisms of the invention may be further engineered to have increased activity of enzymes (e.g., increased activity of enzymes involved in an isobutanol producing metabolic pathway). The term “increased” as used herein with respect to a particular enzymatic activity refers to a higher level of enzymatic activity than that measured in a comparable yeast cell of the same species. For example, overexpression of a specific enzyme can lead to an increased level of activity in the cells for that enzyme. Increased activities for enzymes involved in glycolysis or the isobutanol pathway would result in increased productivity and yield of isobutanol.

Methods to increase enzymatic activity are known to those skilled in the art. Such techniques may include increasing the expression of the enzyme by increased copy number and/or use of a strong promoter, introduction of mutations to relieve negative regulation of the enzyme, introduction of specific mutations to increase specific activity and/or decrease the K_{M} for the substrate, or by directed evolution. See, e.g., Methods in Molecular Biology (vol. 231), ed. Arnold and Georgiou, Humana Press (2003).

### Methods of Using Recombinant Microorganisms for Metabolite Production

For a biocatalyst to produce a beneficial metabolite most economically, it is desirable to produce said metabolite at a high yield. Preferably, the only product produced is the desired metabolite, as extra products (i.e. by-products) lead to a reduction in the yield of the desired metabolite and an increase in capital and operating costs, particularly if the extra products have little or no value. These extra products also require additional capital and operating costs to separate these products from the desired metabolite.

In one aspect, the present application provides methods of producing a desired metabolite using a recombinant described herein. In one embodiment, the recombinant microorganism comprises a KARI-requiring biosynthetic pathway, wherein said recombinant microorganism comprises at least one nucleic acid molecule encoding a KARI that is at least about 80% identical to SEQ ID NO: 2, SEQ ID NO: 4, SEQ ID NO: 6, SEQ ID NO: 8, SEQ ID NO: 10, SEQ ID NO: 28, SEQ ID NO: 40, or SEQ ID NO: 58. In one embodiment, the KARI is derived from the genus Shewanella. In a specific embodiment, the KARI is derived from Shewanella sp. strain MR-4. In another specific embodiment, the isolated nucleic acid molecule is comprised of SEQ ID NO: 1. In another embodiment, the KARI is derived from the genus Vibrio. In a specific embodiment, the KARI is derived from Vibrio fischeri. In another specific embodiment, the isolated nucleic acid molecule is comprised of SEQ ID NO: 3. In yet another embodiment, the KARI is derived from the genus Gramella. In a specific embodiment, the KARI is derived from Gramella forsetii. In another specific embodiment, the isolated nucleic acid molecule is comprised of SEQ ID NO: 5. In yet another embodiment, the KARI is derived from the genus Cytophaga. In a specific embodiment, the KARI is derived from Cytophaga hutchinsonii. In another specific embodiment, the isolated nucleic acid molecule is comprised of SEQ ID NO: 7. In yet another embodiment, the KARI is derived from a genus selected from Lactococcus and Streptococcus. In a specific embodiment, the KARI is derived from Lactococcus lactis, Streptococcus equinus, or Streptococcus infantarius. In another specific embodiment, the KARI is encoded by SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25. In yet another embodiment, the KARI is derived from the genus Methanococcus. In a specific embodiment, the KARI is derived from Methanococcus maripaludis, Methanococcus vannielii, or Methanococcus voltae. In another specific embodiment, the KARI is encoded by SEQ ID NO: 27, SEQ ID NO: 29, SEQ ID NO: 31, SEQ ID NO: 33, SEQ ID NO: 35, or SEQ ID NO: 37. In yet another embodiment, the KARI is derived from a genus selected from Zymomonas, Erythrobacter, Sphingomonas, Sphingobium, and Novosphingobium. In a specific embodiment, the KARI is derived from Zymomonas mobilis, Erythrobacter litoralis, Sphingomonas wittichii, Sphingobium japonicum, Sphingobium chlorophenolicum, or Novosphingobium nitrogenifigens. In another specific embodiment, the KARI is encoded by SEQ ID NO: 39, SEQ ID NO: 41, SEQ ID NO: 43, SEQ ID NO: 45, SEQ ID NO: 47, SEQ ID NO: 49, SEQ ID NO: 51, or SEQ ID NO: 53. In yet another embodiment, the KARI is derived from the genus Bacteroides. In a specific embodiment, the KARI is derived from Bacteroides thetaiotaomicron. In another specific embodiment, the KARI is encoded by SEQ ID NO: 55. In yet another embodiment, the KARI is derived from the genus Schizosaccharomyces. In a specific embodiment, the KARI is derived from Schizosaccharomyces pombe or Schizosaccharomyces japonicus. In another specific embodiment, the KARI is encoded by SEQ ID NO: 57, SEQ ID NO: 59, or SEQ ID NO: 61. In yet another embodiment, the KARI has one or more modifications or mutations at positions corresponding to amino acids selected from: (a) alanine 71 of the Shewanella sp. KARI (SEQ ID NO: 2); (b) arginine 76 of the Shewanella sp. KARI (SEQ ID NO: 2): (c) serine 78 of the Shewanella sp. KARI; and (d) glutamine 110 of the Shewanella sp. KARI (SEQ ID NO: 2). In yet another embodiment, the KARI has one or more modifications or mutations at positions corresponding to amino acids selected from: (a) valine 48 of the L. lactis KARI (SEQ ID NO: 10); (b) arginine 49 of the L. lactis KARI (SEQ ID NO: 10); (c) lysine 52 of the L. lactis KARI (SEQ ID NO: 10); (d) serine 53 of the L. lactis KARI (SEQ ID NO: 10); (e) glutamic acid 59 of the L. lactis KARI (SEQ ID NO: 10): (f); leucine 85 of the L. lactis KARI (SEQ ID NO: 10); (g) isoleucine 89 of the L. lactis KARI (SEQ ID NO: 10); (h) lysine 118 of the L. lactis KARI (SEQ ID NO: 10); (i) threonine 182 of the L. lactis KARI (SEQ ID NO: 10); and (j) glutamic acid 320 of the L. lactis KARI (SEQ ID NO: 10).

In another embodiment, the recombinant microorganism comprises a KARI-requiring biosynthetic pathway, wherein said recombinant microorganism comprises at least one nucleic acid molecule encoding a KARI that is at least about 99% identical to SEQ ID NO: 64. In one embodiment, the KARI is derived from the genus Salmonella. In a specific embodiment, the KARI is derived from Salmonella enterica. In another specific embodiment, the KARI is encoded by SEQ ID NO: 63. In yet another embodiment, the KARI has one or more modifications or mutations at positions corresponding to amino acids selected from: (a) alanine 71 of the S. enterica KARI (SEQ ID NO: 64); (b) arginine 76 of the S. enterica KARI (SEQ ID NO: 64); (c) serine 78 of the S. enterica KARI (SEQ ID NO: 64); (d) glutamine 110 of the S. enterica KARI (SEQ ID NO: 64); (e) aspartic acid 146 of the S. enterica KARI (SEQ ID NO: 64); (f) glycine 185 of the S. enterica KARI (SEQ ID NO: 64); and (g) lysine 433 of the S. enterica KARI (SEQ ID NO: 64).

In an exemplary embodiment, the KARI-requiring biosynthetic pathway is a pathway for the production of a metabolite selected from isobutanol, isoleucine, leucine, valine, pantothenate, coenzyme A, 1-butanol, 2-methyl-1-butanol, 3-methyl-1-butanol, 3-methyl-1-pentanol, 4-methyl-1-pentanol, 4-methyl-1-hexanol, and 5-methyl-1-heptanol. In a further exemplary embodiment, the beneficial metabolite is isobutanol.

In a method to produce a beneficial metabolite (e.g., isobutanol) from a carbon source, the recombinant microorganism is cultured in an appropriate culture medium containing a carbon source. In certain embodiments, the method further includes isolating the beneficial metabolite (e.g., isobutanol) from the culture medium. For example, a beneficial metabolite (e.g., isobutanol) may be isolated from the culture medium by any method known to those skilled in the art, such as distillation, pervaporation, or liquid-liquid extraction. In certain exemplary embodiments, the beneficial metabolite is selected from isobutanol, isoleucine, leucine, valine, pantothenate, coenzyme A, 1-butanol, 2-methyl-1-butanol, 3-methyl-1-butanol, 3-methyl-1-pentanol, 4-methyl-1-pentanol, 4-methyl-1-hexanol, and 5-methyl-1-heptanol. In a further exemplary embodiment, the beneficial metabolite is isobutanol.

In one embodiment, the recombinant microorganism may produce the beneficial metabolite (e.g., isobutanol) from a carbon source at a yield of at least 5 percent theoretical. In another embodiment, the microorganism may produce the beneficial metabolite (e.g., isobutanol) from a carbon source at a yield of at least about 10 percent, at least about 15 percent, about least about 20 percent, at least about 25 percent, at least about 30 percent, at least about 35 percent, at least about 40 percent, at least about 45 percent, at least about 50 percent, at least about 55 percent, at least about 60 percent, at least about 65 percent, at least about 70 percent, at least about 75 percent, at least about 80 percent, at least about 85 percent, at least about 90 percent, at least about 95 percent, or at least about 97.5% theoretical. In a specific embodiment, the beneficial metabolite is isobutanol.

### Distillers Dried Grains Comprising Spent Yeast Biocatalysts

In an economic fermentation process, as many of the products of the fermentation as possible, including the co-products that contain biocatalyst cell material, should have value. Insoluble material produced during fermentations using grain feedstocks, like corn, is frequently sold as protein and vitamin rich animal feed called distillers dried grains (DDG). See, e.g., commonly owned and co-pending U.S. Publication No. 2009/0215137, which is herein incorporated by reference in its entirety for all purposes. As used herein, the term “DDG” generally refers to the solids remaining after a fermentation, usually consisting of unconsumed feedstock solids, remaining nutrients, protein, fiber, and oil, as well as spent yeast biocatalysts or cell debris therefrom that are recovered by further processing from the fermentation, usually by a solids separation step such as centrifugation.

Distillers dried grains may also include soluble residual material from the fermentation, or syrup, and are then referred to as “distillers dried grains and solubles” (DDGS). Use of DDG or DDGS as animal feed is an economical use of the spent biocatalyst following an industrial scale fermentation process.

Accordingly, in one aspect, the present invention provides an animal feed product comprised of DDG derived from a fermentation process for the production of a beneficial metabolite (e.g., isobutanol), wherein said DDG comprise a spent yeast biocatalyst of the present invention. In an exemplary embodiment, said spent yeast biocatalyst has been engineered to comprise at least one nucleic acid molecule encoding a KARI that is at least about 80% identical to SEQ ID NO: 2, SEQ ID NO: 4, SEQ ID NO: 6, SEQ ID NO: 8, SEQ ID NO: 10, SEQ ID NO: 28, SEQ ID NO: 40, or SEQ ID NO: 58. In one embodiment, the KARI is derived from the genus Shewanella. In a specific embodiment, the KARI is derived from Shewanella sp. strain MR-4. In another specific embodiment, the isolated nucleic acid molecule is comprised of SEQ ID NO: 1. In another embodiment, the KARI is derived from the genus Vibrio. In a specific embodiment, the KARI is derived from Vibrio fischeri. In another specific embodiment, the isolated nucleic acid molecule is comprised of SEQ ID NO: 3. In yet another embodiment, the KARI is derived from the genus Gramella. In a specific embodiment, the KARI is derived from Gramella forsetii. In another specific embodiment, the isolated nucleic acid molecule is comprised of SEQ ID NO: 5. In yet another embodiment, the KARI is derived from the genus Cytophaga. In a specific embodiment, the KARI is derived from Cytophaga hutchinsonii. In another specific embodiment, the isolated nucleic acid molecule is comprised of SEQ ID NO: 7. In yet another embodiment, the KARI is derived from a genus selected from Lactococcus and Streptococcus. In a specific embodiment, the KARI is derived from Lactococcus lactis, Streptococcus equinus, or Streptococcus infantarius. In another specific embodiment, the KARI is encoded by SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25. In yet another embodiment, the KARI is derived from the genus Methanococcus. In a specific embodiment, the KARI is derived from Methanococcus maripaludis, Methanococcus vannielii, or Methanococcus voltae. In another specific embodiment, the KARI is encoded by SEQ ID NO: 27, SEQ ID NO: 29, SEQ ID NO: 31, SEQ ID NO: 33, SEQ ID NO: 35, or SEQ ID NO: 37. In yet another embodiment, the KARI is derived from a genus selected from Zymomonas, Erythrobacter, Sphingomonas, Sphingobium, and Novosphingobium. In a specific embodiment, the KARI is derived from Zymomonas mobilis, Erythrobacter litoralis, Sphingomonas wittichii, Sphingobium japonicum, Sphingobium chlorophenolicum, or Novosphingobium nitrogenifigens. In another specific embodiment, the KARI is encoded by SEQ ID NO: 39, SEQ ID NO: 41, SEQ ID NO: 43, SEQ ID NO: 45, SEQ ID NO: 47, SEQ ID NO: 49, SEQ ID NO: 51, or SEQ ID NO: 53. In yet another embodiment, the KARI is derived from the genus Bacteroides. In a specific embodiment, the KARI is derived from Bacteroides thetaiotaomicron. In another specific embodiment, the KARI is encoded by SEQ ID NO: 55. In yet another embodiment, the KARI is derived from the genus Schizosaccharomyces. In a specific embodiment, the KARI is derived from Schizosaccharomyces pombe or Schizosaccharomyces japonicus. In another specific embodiment, the KARI is encoded by SEQ ID NO: 57, SEQ ID NO: 59, or SEQ ID NO: 61. In yet another embodiment, the KARI has one or more modifications or mutations at positions corresponding to amino acids selected from: (a) alanine 71 of the Shewanella sp. KARI (SEQ ID NO: 2); (b) arginine 76 of the Shewanella sp. KARI (SEQ ID NO: 2); (c) serine 78 of the Shewanella sp. KARI; and (d) glutamine 110 of the Shewanella sp. KARI (SEQ ID NO: 2). In yet another embodiment, the KARI has one or more modifications or mutations at positions corresponding to amino acids selected from: (a) valine 48 of the L. lactis KARI (SEQ ID NO: 10); (b) arginine 49 of the L. lactis KARI (SEQ ID NO: 10); (c) lysine 52 of the L. lactis KARI (SEQ ID NO: 10); (d) serine 53 of the L. lactis KARI (SEQ ID NO: 10); (e) glutamic acid 59 of the L. lactis KARI (SEQ ID NO: 10): (f); leucine 85 of the L. lactis KARI (SEQ ID NO: 10); (g) isoleucine 89 of the L. lactis KARI (SEQ ID NO: 10); (h) lysine 118 of the L. lactis KARI (SEQ ID NO: 10); (i) threonine 182 of the L. lactis KARI (SEQ ID NO: 10); and (j) glutamic acid 320 of the L. lactis KARI (SEQ ID NO: 10). In another exemplary embodiment, said spent yeast biocatalyst has been engineered to comprise at least one nucleic acid molecule encoding a KARI that is at least about 99% identical to SEQ ID NO: 64. In one embodiment, the KARI is derived from the genus Salmonella. In a specific embodiment, the KARI is derived from Salmonella enterica. In another specific embodiment, the KARI is encoded by SEQ ID NO: 63. In yet another embodiment, the KARI has one or more modifications or mutations at positions corresponding to amino acids selected from: (a) alanine 71 of the S. enterica KARI (SEQ ID NO: 64); (b) arginine 76 of the S. enterica KARI (SEQ ID NO: 64); (c) serine 78 of the S. enterica KARI (SEQ ID NO: 64); (d) glutamine 110 of the S. enterica KARI (SEQ ID NO: 64); (e) aspartic acid 146 of the S. enterica KARI (SEQ ID NO: 64); (f) glycine 185 of the S. enterica KARI (SEQ ID NO: 64); and (g) lysine 433 of the S. enterica KARI (SEQ ID NO: 64).

In certain additional embodiments, the DDG comprising a spent yeast biocatalyst of the present invention comprise at least one additional product selected from the group consisting of unconsumed feedstock solids, nutrients, proteins, fibers, and oils.

In another aspect, the present invention provides a method for producing DDG derived from a fermentation process using a yeast biocatalyst (e.g., a recombinant yeast microorganism of the present invention), said method comprising: (a) cultivating said yeast biocatalyst in a fermentation medium comprising at least one carbon source; (b) harvesting insoluble material derived from the fermentation process, said insoluble material comprising said yeast biocatalyst; and (c) drying said insoluble material comprising said yeast biocatalyst to produce the DDG.

In certain additional embodiments, the method further comprises step (d) of adding soluble residual material from the fermentation process to said DDG to produce DDGS. In some embodiments, said DDGS comprise at least one additional product selected from the group consisting of unconsumed feedstock solids, nutrients, proteins, fibers, and oils.

This invention is further illustrated by the following examples that should not be construed as limiting. The contents of all references, patents, and published patent applications cited throughout this application, as well as the Figures and the Sequence Listing, are incorporated herein by reference for all purposes.

### Example 1

### Reduction of KARI Inhibition by 2,3-Dihydroxyisovalerate (DHIV)

The purpose of this example is to show how high-performance KARIs were identified.

### Materials and Methods for Example 1

TABLE 2
Strain Used in Examples 1-2.

| | |
|---|---|
| GEVO3956 | MATa ura3 leu2 his3 trp1 ald6::P_{ENO2}- |
| | LI_adhA^{RE1} -P_{FBA1}-Sc_TRP1 |
| | gpd1::T_{KI}_URA3 gpd2::T_{KI}_URA3 tma29::T_{KI}_URA3 |
| | pdc1::P_{PDC1}-LI_kivD2_coSc5-P_{FBA1}- |
| | LEU2-T_{LEU2}-P_{ADH1}-Bs_alsS1_coSc-T_{CYC1}-P_{PGK1}- |
| | LI_kivD2_coEc-P_{ENO2}-Sp_HIS5 pdc5::T_{KI}_URA3 |
| | pdc6::P_{TDH3}-Sc_AFT1-P_{ENO2}-LI_adhA^{RE1}- |
| | T-_{KI}_URA3_short-P_{FBA1}-KI_URA3-T_{KI}_URA3 |

TABLE 3
Plasmid Used in Example 1-2.

| | |
|---|---|
| pGV3009 | P_{Sc}_TEF1:LI_ilvD_coSc:T_{Sc}_ADH1, |
| | P_{Sc}_PDC1-350:EC_ilvC_coSc^{P2D1}_A1_his6, |
| | P_{Sc}_TPI1:G418^{R},P_{Sc}_ENO2:LI_adhA^{RE1}, |
| | CEN/ARS origin of replication, |
| | Ap^{R}, pMB1 origin of replication |

In this example, a series of KARI genes were individually expressed from a yeast promoter in conjunction with other components of an isobutanol production pathway in yeast such that KARI was the limiting enzyme in the pathway and the amount of isobutanol produced during a fermentation was dependent on the KARI activity level. In this system, the S. cerevisiae host strain GEV03956, which expresses ALS and KIVD enzymes, was used to produce isobutanol when supplied with a low copy number plasmid expressing KARI, DHAD, and ADH enzymes.

KARIs were identified and grouped by bioinformatic and phylogenetic methods based on the amino acid sequence. Individual KARIs were chosen for the above analysis to provide a representative sample of broadly diverse clades. KARI genes were designed and synthesized based on the primary amino acid sequence of the chosen KARI, with codon optimization of the genes for expression in S. cerevisiae. These genes were cloned downstream of the Sc_PDC1⁻³⁵⁰ promoter in pGV3009 to replace the Ec_IlvC_coSc^{P2D1-A1}^{_{—}}^{his6} gene present in the plasmid.

Shake Flask Fermentations:

Shake flask fermentations using GEV03956 carrying these individual plasmids were performed together in experiments with GEV03956 carrying pGV3022 (derived from pGV3009 but containing the E. coli ilvC_coSc gene expressed from the Sc_PDC1⁻³⁵⁰ promoter) and GEV03956 carrying pGV3012 (equivalent to pGV3009 lacking the Sc_PDC1⁻³⁵⁰ promoter and KARI gene) for comparison of isobutanol production. The shake flask fermentations were performed as follows. The strains were grown overnight in 3 mL of YPD medium containing 1% v/v ethanol and 0.1 g/L G418 at 30° C. and 250 rpm. The OD₆₀₀ of these cultures was determined after overnight growth and the appropriate amount of culture was added to 50 mL of YP medium containing 5% w/v glucose, 1% v/v ethanol, 200 mM MES, pH 6.5, and 0.1 g/L G418 to obtain an OD₆₀₀ of 0.1 in 250 mL baffled flasks with sleeve caps. Cultures were incubated at 30° C. and 250 rpm overnight. The OD₆₀₀ of these cultures was determined after overnight growth and the appropriate amount of culture to total 250 ODs was added to 50 mL Falcon tubes and centrifuged at 2700×g for 5 minutes. The supernatant was removed and cells were resuspended in 50 mL of YP medium containing 8% w/v glucose, 1% v/v ethanol, 200 mM MES, pH 6.5, and 0.1 g/L G418 to obtain a final OD₆₀₀ of 5 OD per ml. At t=0 the OD₆₀₀ of each flask was determined. The fermentation cultures were incubated at 30° C. and 250 rpm in non-baffled 250 mL flasks with vented screw cap tops. After 24, 48 and 72 hours of incubation, 1.5 mL of culture was removed into 1.5 mL microcentrifuge tubes from each culture. OD₆₀₀ values were determined from the samples and the remainder of each sample was centrifuged for 10 min at 14,000 rpm in a microcentrifuge and 1 mL of the supernatant was removed to be submitted for gas chromatographic analysis. Analysis of volatile organic compounds, including ethanol and isobutanol, was performed on an Agilent 6890 gas chromatograph (GC) fitted with a 7683B liquid autosampler, a split/splitless injector port, a ZB-FFAP column (Phenomenex 30 m length, 0.32 mm ID, 0.25 μM film thickness) connected to a flame ionization detector (FID). The temperature program is as follows: 230° C. for the injector, 300° C. for the detector, 100° C. oven for 1 minute, 35° C./minute gradient to 230° C., and then hold for 2.5 min. Analysis is performed using authentic standards (>98%, obtained from Sigma-Aldrich), and a 6-point calibration curve with 1-pentanol as the internal standard. Injection size is 0.5 μL with a 50:1 split and run time is 7.4 min.

Results:

KARI gene clones resulting in isobutanol production equivalent to or higher than fermentations with pGV3022 or within two standard deviations below that of fermentations with pGV3022, averaged from multiple experiments, were chosen as encoding high-performing KARIs. These fermentations identified the following as high-performance KARIs: Shewanella sp. strain MR-4 (SEQ ID NO: 2), Vibrio fischeri strain ES114 (SEQ ID NO: 4), Gramella forsetii strain KT0803 (SEQ ID NO: 6), and Cytophaga hutchinsonii strain ATCC33406 (SEQ ID NO: 8). Table 4 shows the results of 48 hr and 72 hr isobutanol fermentation timepoints.

TABLE 4
Isobutanol Titers from Fermentations with High-Performance
KARIs as compared to E. coli KARI.

| KARI Gene | 48 h isobutanol | 72 h isobutanol |
|---|---|---|
| Expressed | Titer (g/L) | Titer (g/L) |
| Shewanella sp. MR-4 | 4.01 ± 0.61 | 4.60 ± 0.53 |
| Vibrio fischeri | 4.27 ± 0.27 | 4.52 ± 0.30 |
| Gramella forsetii | 4.38 ± 0.69 | 4.45 ± 0.50 |
| Cytophaga hutchinsonii | 3.49 ± 0.20 | 4.26 ± 0.31 |
| E. coli | 3.93 ± 1.16 | 4.72 ± 0.90 |
| (Mean of 3 experiments ± | | |
| 2 standard deviations) | | |

Each of these identified KARIs share the property of being long-form KARIs. Long-form KARIs are found in plants, algae, and some bacteria, while short-form KARIs are found in fungi and bacteria. The amino acid sequences of these high-performing bacterial long form KARIs were aligned with the sequences of 103 other KARIs representing broad biological diversity of KARIs chosen from the bioinformatic and phylogenetic analysis above and were used to generate a phylogenetic tree in Clone Manager using the “Align Multiple Sequences” feature to perform a Multi-Way alignment of the amino acid sequences using the BLOSUM62 scoring matrix and otherwise default parameters. This analysis identified the Shewanella sp., Vibrio fischeri, Gramella forsetii, Cytophaga hutchinsonii, and E. coli KARIs as all belonging to a distinct clade of closely related bacterial long-form KARIs (see FIG. 2 of U.S. Provisional Application No. 506,562, which is herein incorporated by reference). A separate clade of bacterial long form KARIs contained KARIs from the symbiotic bacteria Buchnera aphidicola and Candidatus blochmannia species.

The Shewanella sp. KARI sequence amino acid sequence (SEQ ID NO: 2) was used to identify the 500 closest protein sequences in the GenBank database using the blastp algorithm on the non-redundant protein sequence database using the default parameters. The COBALT Multiple Alignment link from the BLAST results page was used to perform a multiple alignment of the 500 closest protein sequences plus the Shewanella sp. KARI sequence (SEQ ID NO: 2) using the default parameters. This alignment was downloaded as a “Fasta plus gaps” file and opened with the Clone Manager “Align Multiple Sequences” feature to perform a Multi-Way alignment of the amino acid sequences using the BLOSUM62 scoring matrix and otherwise default parameters to generate a phylogenetic tree of the sequences.

KARI sequences representing the major clades of this tree were chosen and used to generate a representative subset phylogenetic tree. The resulting subset phylogenetic tree showed a clade of proteins sequences containing the Shewanella sp., Vibrio fischeri, Gramella forsetii, Cytophaga hutchinsonii, and E. coli KARIs and KARIs that were closely related to those sequences (see FIG. 3 of U.S. Provisional Application No. 506,562, which is herein incorporated by reference). Each of the Shewanella sp., Vibrio fischeri, Gramella forsetii, Cytophaga hutchinsonii, and E. coli KARIs were in a separate subclade in this tree, indicating that high-performing KARIs for isobutanol production in yeast can be found throughout this overall clade of long-form bacterial KARIs.

### Example 2

The purpose of this example is to show how additional high-performance KARIs were identified.

In this example, a series of KARI genes were individually expressed from a yeast promoter in conjunction with other components of an isobutanol production pathway in yeast such that KARI was the limiting enzyme in the pathway and the amount of isobutanol produced during a fermentation was dependent on the KARI activity level. In this system, the S. cerevisiae host strain GEV03956, which expresses ALS and KIVD enzymes, was used to produce isobutanol when supplied with a low copy number plasmid expressing KARI, DHAD, and ADH enzymes.

KARIs were identified and grouped by bioinformatic and phylogenetic methods based on the amino acid sequence. Individual KARIs were chosen for the above analysis to provide a representative sample of broadly diverse clades. KARI genes were designed and synthesized based on the primary amino acid sequence of the chosen KARI, with codon optimization of the genes for expression in S. cerevisiae. These genes were cloned downstream of the Sc_PDC1⁻³⁵⁰ promoter in pGV3009 to replace the Ec_IlvC_coSc^{P2D-A1}^{_{—}}^{his6} gene present in the plasmid.

Shake Flask Fermentations:

Shake flask fermentations using GEV03956 carrying these individual plasmids were performed together in experiments with GEV03956 carrying pGV3022 (derived from pGV3009 but containing the E. coli ilvC_coSc gene expressed from the Sc_PDC1⁻³⁵⁰ promoter) and GEV03956 carrying pGV3012 (equivalent to pGV3009 lacking the Sc_PDC1⁻³⁵⁰ promoter and KARI gene) for comparison of isobutanol production. The shake flask fermentations were performed as follows. The strains were grown overnight in 3 mL of YPD medium containing 1% v/v ethanol and 0.1 g/L G418 at 30° C. and 250 rpm. The OD₆₀₀ of these cultures was determined after overnight growth and the appropriate amount of culture was added to 50 mL of YP medium containing 5% w/v glucose, 1% v/v ethanol, 200 mM MES, pH 6.5, and 0.1 g/L G418 to obtain an OD₆₀₀ of 0.1 in 250 mL baffled flasks with sleeve caps. Cultures were incubated at 30° C. and 250 rpm overnight. The OD₆₀₀ of these cultures was determined after overnight growth and the appropriate amount of culture to total 250 ODs was added to 50 mL Falcon tubes and centrifuged at 2700×g for 5 minutes. The supernatant was removed and cells were resuspended in 50 mL of YP medium containing 8% w/v glucose, 1% v/v ethanol, 200 mM MES, pH 6.5, and 0.1 g/L G418 to obtain a final OD₆₀₀ of 5 OD per ml. At t=0 the OD₆₀₀ of each flask was determined. The fermentation cultures were incubated at 30° C. and 250 rpm in non-baffled 250 mL flasks with vented screw cap tops. After 24, 48 and 72 hours of incubation, 1.5 mL of culture was removed into 1.5 mL microcentrifuge tubes from each culture. OD₆₀₀ values were determined from the samples and the remainder of each sample was centrifuged for 10 min at 14,000 rpm in a microcentrifuge and 1 mL of the supernatant was removed to be submitted for gas chromatographic analysis. Analysis of volatile organic compounds, including ethanol and isobutanol, was performed on an Agilent 6890 gas chromatograph (GC) fitted with a 7683B liquid autosampler, a split/splitless injector port, a ZB-FFAP column (Phenomenex 30 m length, 0.32 mm ID, 0.25 μM film thickness) connected to a flame ionization detector (FID). The temperature program is as follows: 230° C. for the injector, 300° C. for the detector, 100° C. oven for 1 minute, 35° C./minute gradient to 230° C., and then hold for 2.5 min. Analysis is performed using authentic standards (>98%, obtained from Sigma-Aldrich), and a 6-point calibration curve with 1-pentanol as the internal standard. Injection size is 0.5 μL with a 50:1 split and run time is 7.4 min.

Results:

KARI gene clones resulting in isobutanol production equivalent to or higher than fermentations with pGV3022 or within two standard deviations below that of fermentations with pGV3022, averaged from multiple experiments, were chosen as encoding high-performing KARIs. These fermentations identified the following as high-performance KARIs: Lactococcus lactis strain KF147 (SEQ ID NO: 10), Methanococcus maripaludis strain C5 (SEQ ID NO: 28), Zymomonas mobilis strains ZM4, ATCC10988, and NCIMB 11163 (SEQ ID NO: 40), Bacteroides thetaiotaomicron strain VPI-5482 (SEQ ID NO: 56), and Schizosaccharomyces pombe strain ATCC33406 (SEQ ID NO: 58). Table 5 shows the results of 48 hr and 72 hr isobutanol fermentation timepoints.

TABLE 5
Isobutanol Titers from Fermentations with High-Performance
KARIs as compared to E. coli KARI.

| KARI Gene | 48 h Isobutanol | 72 h Isobutanol |
|---|---|---|
| Expressed | Titer (g/L) | Titer (g/L) |
| Lactococcus lactis | 5.68 ± 0.90 | 6.21 ± 0.62 |
| Methanococcus maripaludis | 4.03 ± 0.18 | 4.70 ± 0.18 |
| Zymomonas mobilis | 4.00 ± 0.32 | 4.67 ± 0.24 |
| Bacteroides thetaotaomicron | 3.21 ± 0.03 | 4.07 ± 0.01 |
| Schizosaccharomyces pombe | 3.33 ± 0.30 | 3.89 ± 0.23 |
| (Δ54tr)* | | |
| E. coli | 3.93 ± 1.16 | 4.72 ± 0.90 |
| (Mean of 3 experiments ± | | |
| 2 standard deviations) | | |

*Truncated by removal of first 54 AA encoding MTS (SEQ ID NO: 90).

Each of these identified KARIs share the property of being short-form KARIs. Short-form KARIs are found in fungi and bacteria, while long-form KARIs are found in plants, algae, and some bacteria. An additional 21 short-form KARIs tested did not meet the yeast isobutanol fermentation criteria in the above experiments.

The Lactococcus lactis, Methanococcus maripaludis, and Zymomonas mobilis KARIs were also identified as performing as well or better than the E. coli KARI in shake flask fermentations when expressed from a high copy number plasmid. Genes encoding these KARIs were cloned downstream of a Sc_TDH3 promoter to replace the Ec_ilvC_coSc^{P2D1-A1}^{_{—}}^{his6} gene present in that plasmid.

Shake flask fermentations of GEVO3956 carrying these individual plasmids were performed together with GEVO3956 carrying pGV2911 (derived from pGV2901 but containing the E. coli ilvC_coSc gene expressed from the Sc_TDH3 promoter) for comparison of isobutanol production. The shake flask fermentations were performed as follows. The strains were grown overnight in 3 mL of YPD medium containing 1% v/v ethanol and 0.1 g/L G418 at 30° C. and 250 rpm. The OD₆₀₀ of these cultures was determined after overnight growth and the appropriate amount of culture was added to 50 mL of YP medium containing 5% w/v glucose, 1% v/v ethanol, 200 mM MES, pH 6.5, and 0.1 g/L G418 to obtain an OD₆₀₀ of 0.1 in 250 mL baffled flasks with sleeve caps. Cultures were incubated at 30° C. and 250 rpm overnight. The OD₆₀₀ of these cultures was determined after overnight growth and the appropriate amount of culture to total 250 ODs was added to 50 mL Falcon tubes and centrifuged at 2700×g for 5 minutes. The supernatant was removed and cells were resuspended in 50 mL of YP medium containing 8% w/v glucose, 1% v/v of a stock of 3 g/L ergosterol and 132 g/L Tween 80 dissolved in ethanol, 200 mM MES, pH 6.5, and 0.2 g/L G418 to obtain a final OD₆₀₀ of 5 OD per ml. At t=0 the OD₆₀₀ of each flask was determined. The fermentation cultures were incubated at 30° C. and 250 rpm in non-baffled 250 mL flasks with vented screw cap tops. After 24, 46-48 and 72 hours of incubation, 1.5 mL of culture was removed into 1.5 mL microcentrifuge tubes from each culture. OD₆₀₀ values were determined from the samples and the remainder of each sample was centrifuged for 10 min at 14,000 rpm in a microcentrifuge and 1 mL of the supernatant was removed to be submitted for gas chromatographic analysis. Analysis of volatile organic compounds, including ethanol and isobutanol, was performed on an Agilent 6890 gas chromatograph (GC) fitted with a 7683B liquid autosampler, a split/splitless injector port, a ZB-FFAP column (Phenomenex 30 m length, 0.32 mm ID, 0.25 μM film thickness) connected to a flame ionization detector (FID). The temperature program is as follows: 230° C. for the injector, 300° C. for the detector, 100° C. oven for 1 minute, 35° C./minute gradient to 230° C., and then hold for 2.5 min. Analysis is performed using authentic standards (>98%, obtained from Sigma-Aldrich), and a 6-point calibration curve with 1-pentanol as the internal standard. Injection size is 0.5 μL with a 50:1 split and run time is 7.4 min.

The M. maripaludis, Z. mobilis, and L. lactis KARIs, expressed from plasmids in GEV03956, resulted in isobutanol titers within two standard deviations of that produced from pGV2911 in GEV03956 at both the 48 hour and 72 hour fermentation time points (Tables 6 and 7).

TABLE 6
Isobutanol titers from isobutanol fermentations with the
M. maripaludis and Z. mobilis KARIs versus the E. coli KARI
expressed from high copy number plasmids.

| KARI Gene | 48 h Isobutanol | 72 h Isobutanol |
|---|---|---|
| Expressed | Titer (g/L) | Titer (g/L) |
| M. maripaludis | 11.26 ± 0.84 | 12.60 ± 1.08 |
| Z. mobilis | 11.47 ± 0.07 | 13.13 ± 0.02 |
| E. coli | 12.34 ± 1.06 | 13.61 ± 1.06 |
| (Mean of 3 experiments ± | | |
| 2 standard deviations) | | |

TABLE 7
Isobutanol titers from isobutanol fermentations
with the L. lactis KARI versus the E. coli KARI
expressed from high copy number plasmids.

| KARI Gene | 48 h Isobutanol | 72 h Isobutanol |
|---|---|---|
| Expressed | Titer (g/L) | Titer (g/L) |
| L. lactis | 7.25 ± 0.88 | 14.51 ± 2.16 |
| E. coli | 7.39 ± 2.64 | 13.54 ± 4.54 |
| (Mean of 3 experiments ± | | |
| 2 standard deviations) | | |

The L. lactis, M. maripaludis, Z. mobilis, B. thetaiotaomicron, and S. pombe KARI amino acid sequences were used to identify the closest protein sequences in the GenBank database using the blastp algorithm on the non-redundant protein sequence database using the default parameters. Table 8 discloses KARI sequences that have ≧80% amino acid identity with the L. lactis, M. maripaludis, Z. mobilis, or S. pombe KARIs.

TABLE 8
KARI sequences having ≧ 80% amino acid identity with the
L. lactis, M. maripaludis, Z. mobilis, or S. pombe KARIs.

| Reference Sequence | Origin | SEQ ID NO: |
|---|---|---|
| L. lactis subsp. lactis | L. lactis subsp. lactis II1430 | 12 |
| KF147 | L. lactis subsp. lactis CV56 | 14 |
| (SEQ ID NO: 10) | L. lactis subsp. cremoris MG1363 | 16 |
| | L. lactis subsp. cremoris NZ9000 | 18 |
| | L. lactis subsp. cremoris SK11 | 20 |
| | L. lactis subsp. lactis NCDO2118 | 22 |
| | S. equinus ATCC 9812 | 24 |
| | S. infantarius subsp. infantarius | 26 |
| | ATCC BAA-102 | |
| M. maripaludis | M. maripaludis C7 | 30 |
| strain C5 | M. maripaludis C6 | 32 |
| (SEQ ID NO: 28) | M. maripaludis S2 | 34 |
| | M. vannielii SB | 36 |
| | M. voltae A3 | 38 |
| Z. mobilis strain | Erythrobacter sp. NAP1 | 42 |
| ZM4 | Sphingomonas wittichii RW1 | 44 |
| (SEQ ID NO: 40) | Sphingobium japonicum UT26S | 46 |
| | Erythrobacter litoralis HTCC2594 | 48 |
| | Sphingobium chlorophenolicum L-1 | 50 |
| | Sphingomonas sp. S17 | 52 |
| | Novosphingobium nitrogenifigens | 54 |
| | DSM 19370 | |
| S. pombe strain | S. pombe PR745 | 60 |
| 972 h-* (Fuii Length: | S. japonicus yFS275 | 62 |
| SEQ ID NO: 58) | | |

A phylogenetic tree that discloses the database identification numbers of 50 KARI sequences that have 278% amino acid identity with the B. thetaiotaomicron KARI and their phylogenetic relationship with the B. thetaiotaomicron KARI was generated (see FIG. 2 of U.S. Provisional Application No. 506,564, which is herein incorporated by reference).

The alignments of the B. thetaiotaomicron KARI protein with 42 closely related KARIs (85-97% amino acid sequence identity in aligned regions) from other Bacteroides strains and species indicates that the N-terminal 12 amino acids of the B. thetaiotaomicron KARI are not conserved and are missing from these related proteins. There is no clearly identifiable ribosome binding site with appropriate spacing upstream of either the annotated start codon of the B. thetaiotaomicron KARI gene sequence annotated from the B. thetaiotaomicron genome sequence project (NCBI reference sequence NC_{—}004663) or the methionine codon for amino acid position 13 of the annotated protein (nucleotide positions 2600124-2600122 from NCBI reference sequence NC_{—}004663). As such it is difficult to determine whether the start codon in B. thetaiotaomicron is at the annotated position (nucleotides 2600160-2600158 from NCBI reference sequence NC_{—}004663) or at the methionine codon for amino acid position 13 of the annotated protein (nucleotide positions 2600124-2600122 from NCBI reference sequence NC_{—}004663). Based on these analyses, a version of the B. thetaiotaomicron KARI lacking the N-terminal 12 amino acids of the annotated protein may function as well or better for isobutanol production in yeast compared with performance of the B. thetaiotaomicron KARI. Such a protein would have the sequence of SEQ ID NO: 88.

### Example 3

### Cofactor Switch of the L. lactis KARI

The purpose of this example is to demonstrate how the cofactor specificity of the L. lactis KARI can be switched from NADPH to NADH.

Similar to all known native KARI enzymes, the L. lactis KARI is NADPH-dependent. To enable the enzyme's use in the production of isobutanol at theoretical yield and/or under anaerobic conditions, the enzyme's cofactor usage was switched from NADPH to NADH.

### Materials and Methods for Example 3

TABLE 9
Strains Used in Example 3.

| Strain | Genotype/Source |
|---|---|
| E. coli | F⁻ ompT gal dcm lon hsdS_{B}(r_{B}⁻m_{B}⁻) λ (DE3 |
| BL21 (DE3) | [lacI lacUV5-T7 gene 1 ind1 sam7 nin5] |

TABLE 10
Plasmids Used in Example 3.

| Plasmid | Genotype |
|---|---|
| pET22b(+) | PT7, bla, ori pBR322, lacI, C-term 6xHis |
| pET[ilvC] | PT7::Ec_ilvC_coEc^{his6}, bla, oripBR322, lacI |
| pGV3281 | PT7::LI_KARI_coSc^{his6}, bla, oripBR322, lacI |
| pETLI1A9 | PT7::LI_KARI^{1A9}_coSc^{his6}, bla, opripBR322, lacI |
| pETLI1G2 | PT7::LI_KARI^{1G2}_coSc^{his6}, bla, oripBR322, lacI |
| pETLI1C2 | PT7::LI_KARI^{1C2}_coSc^{his6}, bla, oripBR322, lacI |
| pETLI1G5 | PT7::LI_KARI^{1G5}_coSc^{his6}, bla, oripBR322, lacI |
| pETLI4H8 | PT7::LI_KARI^{4H8}_coSc^{his6}, bla, oripBR322, lacI |
| pETLI3C7 | PT7::LI_KARI^{3C7}_coSc^{his6}, bla, oripBR322, lacI |
| pETLINKRGen6a | PT7::LI_NKR^{Gen6a}_coSc^{his6}, bla, oripBR322, lacI |
| pETLINKRGen6b | PT7::LI_NKR^{Gen6b}_coSc^{his6}, bla, oripBR322, lacI |

TABLE 11
Primers Used in Example 3.

| # | Primer name | Sequence |
|---|---|---|
| 1 | T7_for | TAATACGACTCACTATAGGG |
| | | (SEQ ID NO: 91) |
| 2 | T7_rev | GCTAGTTATTGCTCAGCGG |
| | | (SEQ ID NO: 92) |
| 3 | LIKARI_Y26NNK_for | ATCGCCGTTATTGGANNKGG |
| | | TTCACAAGGACATGCCCATG |
| | | (SEQ ID NO: 93) |
| 4 | LIKARI_Y26NNK_rev | CATGGGCATGTCCTTGTGAA |
| | | CCMNNTCCAATAACGGCGAT |
| | | (SEQ ID NO: 94) |
| 5 | LIKARI_V48NNK_for | CAATGTTATCATTGGTNNKA |
| | | GGCACGGAAAATCTTTTGAT |
| | | (SEQ ID NO: 95) |
| 6 | LIKARI_V48NNK_rev | ATCAAAAGATTTTCCGTGCC |
| | | TMNNACCAATGATAACATTG |
| | | (SEQ ID NO: 96) |
| 7 | LIKARI_R49NNK_for | GTTATCATTGGTGTANNKCA |
| | | CGGAAAATCTTTTG |
| | | (SEQ ID NO: 97) |
| 8 | LIKARI_R49NNK_rev | CAAAAGATTTTCCGTGMNNT |
| | | ACACCAATGATAAC |
| | | (SEQ ID NO: 98) |
| 9 | LIKARI_G51NNK_for | ATTGGTGTAAGGCACNNKAA |
| | | ATCTTTTGATAAAGCTAAG |
| | | (SEQ ID NO: 99) |
| 10 | LIKARI_G51NNK_rev | CTTAGCTTTATCAAAAGATTT |
| | | MNNGTGCCTTACACCAAT |
| | | (SEQ ID NO: 100) |
| 11 | LIKARI_K52NNK_for | GGTGTAAGGCACGGANNKTC |
| | | TTTTGATAAAGCTAAGGAA |
| | | (SEQ ID NO: 101) |
| 12 | LIKARI_K52NNK_rev | TTCCTTAGCTTTATCAAAAG |
| | | AMNNTCCGTGCCTTACACC |
| | | (SEQ ID NO: 102) |
| 13 | LIKARI_S53NNK_for | GTGTAAGGCACGGAAAANNK |
| | | TTTGATAAAGCTAAGGA |
| | | (SEQ ID NO: 103) |
| 14 | LIKARI_S53NNK_rev | TCCTTAGCTTTATCAAAMNN |
| | | TTTTCCGTGCCTTACAC |
| | | (SEQ ID NO: 104) |
| 15 | LIKARI_L85NNK_for | TTTGGCACCAGATGAGNNKC |
| | | AACAATCCATATACGAG |
| | | (SEQ ID NO: 105) |
| 16 | LIKARI_L85NNK_rev | CTCGTATATGGATTGTTGMN |
| | | NCTCATCTGGTGCCAM |
| | | (SEQ ID NO: 106) |
| 17 | LIKARI_I89NNK_for | GAGTTGCAACAATCCNNKTA |
| | | CGAGGAGGATATCAAGCCT |
| | | (SEQ ID NO: 107) |

| | | |
|---|---|---|
| 18 | LIKARI_I89NNK_rev | AGGCTTGATATCCTCCTCGT |
| | | AMNNGGATTGTTGCAACTC |
| | | (SEQ ID NO: 108) |
| 19 | LI_recomb_1a_for | GGGCACAATGTTATCATTGG |
| | | TSYACBACACGGAMWATCTT |
| | | TTGATAAAGCTAAGGAAG |
| | | (SEQ ID NO: 109) |
| 20 | LI_recomb_1b_for | GGGCACAATGTTATCATTGG |
| | | TSYAGTGCACGGAMWATCTT |
| | | TTGATAAAGCTAAGGAAG |
| | | (SEQ ID NO: 110) |
| 21 | LI_recomb_1c_for | GGGCACAATGTTATCATTGG |
| | | TSYATCGCACGGAMWATCTT |
| | | TTGATAAAGCTAAGGAAG |
| | | (SEQ ID NO: 111) |

| | | |
|---|---|---|
| 22 | LI_recomb_1a_rev | CTTCCTTAGCTTTATCAAAA |

| | | |
|---|---|---|
| | | GATWKTCCGTGTVGTRSAC |
| | | CAATGATAACATTGTGCCC |
| | | (SEQ ID NO: 112) |
| 23 | LI_recomb_1b_rev | CTTCCTTAGCTTTATCAAAA |
| | | GATWKTCCGTGCACTRSACC |
| | | AATGATAACATTGTGCCC |
| | | (SEQ ID NO: 113) |
| 24 | LI_recomb_1c_rev | CTTCCTTAGCTTTATCAAAA |
| | | GATWKTCCGTGCGATRSACC |
| | | AATGATAACATTGTGCCC |
| | | (SEQ ID NO: 114) |
| 25 | LI_recomb_2a_for | GGCACCAGATGAGRCACAAC |
| | | AATCCATATACGAGGAGGAT |
| | | ATCAAGCC |
| | | (SEQ ID NO: 115) |
| 26 | LI_recomb_2b_for | GGCACCAGATGAGRCACAAC |
| | | AATCCGCATACGAGGAGGAT |
| | | ATCAAGCC |
| | | (SEQ ID NO: 116) |
| 27 | LI_recomb_2c_for | GGCACCAGATGAGTTGCAAC |
| | | AATCCATATACGAGGAGGAT |
| | | ATCAAGCC |
| | | (SEQ ID NO: 117) |
| 28 | LI_recomb_2d_for | GGCACCAGATGAGTTGCAAC |
| | | AATCCGCATACGAGGAGGAT |
| | | ATCAAGCC |
| | | (SEQ ID NO: 118) |
| 29 | LI_recornb_2a_rev | GGCTTGATATCCTCCTCGTA |
| | | TATGGATTGTTGTGYCTCAT |
| | | CTGGTGCC |
| | | (SEQ ID NO: 119) |
| 30 | LI_recomb_2b_rev | GGCTTGATATCCTCCTCGTA |
| | | TGCGGATTGTTGTGYCTCAT |
| | | CTGGTGCC |
| | | (SEQ ID NO: 120) |
| 31 | LI_recomb_2c_rev | GGCTTGATATCCTCCTCGTA |
| | | TATGGATTGTTGCAACTCAT |
| | | CTGGTGCC |
| | | (SEQ ID NO: 121) |
| 32 | LI_recomb_2d_rev | GGCTTGATATCCTCCTCGTA |
| | | TGCGGATTGTTGCAACTCAT |
| | | CTGGTGCC |
| | | (SEQ ID NO: 122) |
| 33 | LI_recomb_3KS_for | CACGGAAAATCTTTTGATAA |
| | | AGCTAAGGAA |
| | | (SEQ ID NO: 123) |
| 34 | LI_recomb_3LS_for | CACGGACTATCTTTTGATAA |
| | | AGCTAAGGAA |
| | | (SEQ ID NO: 124) |
| 35 | LI_recomb_3KD_for | CACGGAAAAGATTTTGATAA |
| | | AGCTAAGGAA |
| | | (SEQ ID NO: 125) |
| 36 | LI_recomb_3LD_for | CACGGACTAGATTTTGATAA |
| | | AGCTAAGGAA |
| | | (SEQ ID NO: 126) |
| 37 | LI_recomb_3KS_rev | TTCCTTAGCTTTATCAAAAG |
| | | ATTTTCCGTG |
| | | (SEQ ID NO: 127) |
| 38 | LI_recomb_3LS_rev | TTCCTTAGCTTTATCAAAAG |
| | | ATAGTCCGTG |
| | | (SEQ ID NO: 128) |
| 39 | LI_recomb_3KD_rev | TTCCTTAGCTTTATCAAAAT |
| | | CTTTTCCGTG |
| | | (SEQ ID NO: 129) |
| 40 | LI_recomb_3LD_rev | TTCCTTAGCTTTATCAAAAT |
| | | CTAGTCCGTG |
| | | (SEQ ID NO: 130) |
| 41 | LI_K52NNkS53NNK_for | GGTCTACCACACGGANNKNN |
| | | KTTTGATAAAGCTAAG |
| | | (SEQ ID NO: 131) |
| 42 | LI_K52NNkS53NNK_rev | CTTAGCTTTATCAAAMNNMN |
| | | NTCCGTGTGGTAGACC |
| | | (SEQ ID NO: 132) |
| 43 | E59K_recomb_rev | AAAAGTTTCGAATCCATCTT |
| | | YCTTAGCTTTATC |
| | | (SEQ ID NO: 133) |
| 44 | E59K_recomb_for | GATAAAGCTAAGRAAGATGG |
| | | ATTCGAAACTTTT |
| | | (SEQ ID NO: 134) |
| 45 | A70V_recomb_rev | ATCTGCCTTAGCTACTRCTT |
| | | CACCTACTTCAAA |
| | | (SEQ ID NO: 135) |
| 46 | A70V_recomb_for | TTTGAAGTAGGTGAAGYAGT |
| | | AGCTAAGGCAGAT |
| | | (SEQ ID NO: 136) |
| 47 | K118E/D122G_recomb_for | GGATACATCRAAGTCCCAGA |
| | | GGRCGTGGACGTGTTTATG |
| | | (SEQ ID NO: 137) |
| 48 | K118E/D122G_recomb_rev | CATAAACACGTCCACGYCCT |
| | | CTGGGACTTYGATGTATCC |
| | | (SEQ ID NO: 138) |
| 49 | H135L_recomb_rev | GGTCCTTCTAACAAGGWGGC |
| | | CTGGTGCTTTTGG |
| | | (SEQ ID NO: 139) |
| 50 | H135L_recomb_for | CCAAAAGCACCAGGCCWCCT |
| | | TGTTAGAAGGACC |
| | | (SEQ ID NO: 140) |
| 51 | T182S_recomb_rev | CTCTTCCTTGAAAGTGSTTT |
| | | CAATGATGCCGAC |
| | | (SEQ ID NO: 141) |
| 52 | T182S_recomb_for | GTCGGCATCATTGAAASCAC |
| | | TTTCAAGGAAGAG |
| | | (SEQ ID NO: 142) |
| 53 | E320K_recomb_rev | CATAGCTTGTCTAAGTTYTG |
| | | CCCCTATCTTTTC |
| | | (SEQ ID NO: 143) |
| 54 | E320K_recomb_for | GAAAAGATAGGGGCARAACT |
| | | TAGACAAGCTATG |
| | | (SEQ ID NO: 144) |

* A (Adenine), G (Guanine), C (Cytosine), T (Thymine), R (Purine - A or G), Y (Pyrimidine - C or T), N (Any nucleotide), S (Strong - G or C), M (Amino - A or C), K (Keto - G or T), B (Not A - C, G, or T), W (Weak - A or T), V (Not T - A, C, or G)

Heterologous Expression of Wild-Type L. lactis KARI in E. coli:

Expression of wild-type L. lactis KARI was conducted in a 2-L baffled Erlenmeyer flask filled with 1 L LBₐₘₚ (Luria Bertani Broth, Research Products International Corp, supplemented with 100 μg/mL ampicillin) inoculated with overnight culture to an initial OD₆₀₀ of 0.1. After growing the expression culture at 37° C. with shaking at 250 rpm for 4 h, the cultivation temperature was dropped to 25° C., and KARI expression was induced with IPTG to a final concentration of 0.5 mM. After 24 h at 25° C. and shaking at 250 rpm, the cells were pelleted at 5,300 g for 10 min and then frozen at −20° C. until further use.

Heterologous Expression of L. lactis KARI Variants in E. coli:

The expression of L. lactis KARI variants was conducted in 0.25-L Erlenmeyer flasks filled with 50 mL LBₐₘₚ (Luria Bertani Broth, Research Products International Corp, supplemented with 100 μg/mL ampicillin) inoculated with overnight culture to an initial OD₆₀₀ of 0.1. After growing the expression cultures at 37° C. with shaking at 250 rpm for 4 h, the cultivation temperature was dropped to 25° C., and KARI expression was induced with IPTG to a final concentration of 0.5 mM. After 24 h at 25° C. and shaking at 250 rpm, the cells were pelleted at 5,300 g for 10 min and then frozen at −20° C. until further use.

Histrap Purification of L. lactis KARI: L. lactis KARI was purified over a 5-mL histrap column.

Histrap Purification of L. lactis KARI Variants:

L. lactis KARI variants were purified over 1-mL histrap columns.

Preparation of Enantiopure (S)-2-Acetolactate:

Enzymatic synthesis of (S)-2-acetolactate was performed in an anaerobic flask. The reaction was carried out in a total volume of 55 mL containing 20 mM potassium phosphate buffer, pH 7.0, 1 mM MgCl₂, 0.05 mM thiamine pyrophosphate (TPP), and 200 mM sodium pyruvate. The synthesis was initiated by the addition of 65 units of purified B. subtilis acetolactate synthase (Bs_AlsS), and the reaction was incubated at 30° C. (in a static incubator) for 7.5 hours. A buffer exchange was performed on the purified Bs_AlsS before the synthesis to remove as much glycerol as possible. This was done using a microcon filter with a 50 kDa nominal molecular weight cutoff membrane to filter 0.5 mL of the purified enzyme until only 50 μL were left on top of the membrane. 450 μL of 20 mM KPO₄ pH 7.0, 1 mM MgCl₂, and 0.05 mM TPP were then added to the membrane and filtered again; this process was repeated three times. The final acetolactate concentration was determined by liquid chromatography and was ˜200 mM.

KARI Assay in 1-mL Scale to Measure NADPH and NADH K_{M} Values:

L. lactis KARI activity or activities of its variants were assayed kinetically by monitoring the decrease in NADPH or NADH concentration by measuring the change in absorbance at 340 nm. An assay buffer was prepared containing 100 mM potassium phosphate pH 7.0, 1 mM DTT. 2.5 mM (S)-2-acetolactate, and 10 mM MgCl₂ (final concentrations in the 1-mL assay, accounting for dilution with enzyme and cofactor). Fifty μL purified enzyme and 930 μL of the assay buffer were placed into a 1-mL cuvette. The reaction was initiated by addition of 20 μL NADPH or NADH (200 μM final concentration) for a general activity assay. Michaelis-Menten constants of the cofactors were determined with varying concentrations of NADPH (500—12 μM final) or NADH (200—6 μM final).

Construction of Site-Saturation Libraries (Generation 1):

One-site site-saturation libraries (with NNK codons) were constructed using standard SOE PCR with Phusion polymerase, pGV3281 as template, and respective primer pairs #1-18. The fragments were DpnI digested for 1 h, separated on an agarose gel, freeze'n'squeeze (BIORAD) treated, and finally precipitated with pellet paint (Novagen). The clean fragments served as templates for the assembly PCRs using commercial T7 forward and reverse primers (primers #1 and 2) as flanking primers. After successful assembly, the insert was restriction digested with NdeI and XhoI, ligated into pET22b(+), and electro-competent BL21(D3) cells (Lucigen) were transformed.

Construction of Recombination Library (Generation 2):

The recombination library was constructed using SOE PCR introducing mutations found at eight target sites while allowing for the respective wild-type residues as well. We generated four fragments using pGV3281 as template and primers #1, 2, and 19-32. Primers 19 through 21, 22-24, 25-28, and 29-32 were mixed manually to give equimolar distributions of the codons they contained. The fragments were DpnI digested for 1 h at 37° C., separated on an agarose gel, freeze'n'squeezed (BIORAD) and finally pellet painted (Novagen). The fragments served as templates in the assembly PCR using commercial T7 forward and reverse primers as flanking primers. The purified assembly product (Zymo clean up) was restriction digested with NdeI and XhoI, ligated into pET22b(+), and electro-competent BL21(D3) cells (Lucigen) were transformed. Although the S53D mutation had not been identified as beneficial in our NNK libraries, it was included in the recombination library (=library 2). An assembly product of the recombination library described above was used to introduce S53D via SOE PCR using primer pairs 33-40 (Table 11). The forward and reverse primers were mixed manually as described above. The resulting fragments were gel purified and used as templates for the assembly PCR with flanking T7 primers. The resulting assembly PCR product was treated as described above.

Construction of Double NNK Library (K52NNKS53NNK) (Generation 3):

The double NNK library was constructed via SOE PCR using construct pETLI1G2 as template and primers #1 and 2, and 41 and 42. The construction of the library was as described above (site-saturation library). 2,800 colonies were picked for screening.

High-Throughput Expression of L. lactis KARI Variants in E. coli:

For growth and expression of KARI variants in deep well plates, sterile toothpicks were used to pick single colonies into shallow 96-well plates filled with 300 μL LBₐₘₚ. Fifty μL of these overnight cultures were used to inoculate deep well plates filled with 600 μL of LBₐₘₚ per well. The plates were grown at 37° C. with shaking at 250 rpm for 3 h. One hour before induction with IPTG (final concentration 0.5 mM), the temperature of the incubator was reduced to 25° C. After induction, growth and expression continued for 20 h at 25° C. and 250 rpm. Cells were harvested at 5.300 g and 4° C. and then stored at −20° C. The plates always contained four wild-type or parent L. lactis KARI colonies, three BL21(DE3) colonies carrying pET22b(+) to control for background reactions in cell lysates, and one well that contained only media to make sure the plates were free of contaminations.

High-Throughput Screening:

Frozen cell pellets were thawed at room temperature for 20 min and then 200 μL of lysis buffer (100 mM Kpi, 750 mg/L lysozyme, 10 mg/L DNaseI, pH 7) were added. Plates were vortexed to resuspend the cell pellets. After a 60 min incubation phase at 37° C. and shaking at 130 rpm, plates were centrifuged at 5,300 g and 4° C. for 10 min. Forty μL of the resulting crude extracts were transferred into assay plates (flat bottom, Rainin) using a liquid handling robot. Twenty mL assay buffer per plate were prepared (100 mM Kpi, pH 7, 2.5 mM (S)-2-acetolactate, 1 mM DTT, 200 μM NADPH or NADH, and 10 mM MgCl₂) and 160 μL thereof were added to each well to start the reaction resulting in a 20% dilution of the ingredients. The depletion of NAD(P)H was monitored at 340 nm in a plate reader (TECAN) over 200 s.

Results:

The residues chosen to test by site-saturation mutagenesis were Y25, V48, R49, G51, S53, L85, and 189 of the L. lactis KARI (SEQ ID NO: 2). Site-saturation libraries were constructed as described in the materials and method section. After successful transformation of BL21(DE3) cells, 88 individual clones per library were chosen. The libraries were screened with NADH (not NADPH) as cofactor. Screening results are summarized in Table 12.

TABLE 12
Exemplary Variants of
Generation 1 NNK Libraries.

| L. lactic | Beneficial | % improvement |
|---|---|---|
| KARI Sites | Mutations | over parent |
| Y25 | none | n/a |
| V48 | P | 73% |
| | V | 44% |
| | L | 80% |
| R49 | S | 67% |
| | P | 60% |
| G51 | none | n/a |
| K52 | L | 24% |
| S53 | none | n/a |
| L85 | T | 61% |
| | A | 61% |
| I89 | A | 43% |

No improved variants were found in libraries harboring Y25, G51, and S53 mutations.

Recombination Libraries:

Generation 2: A recombination library introducing all mutations found at each site (Table 12) was constructed and also allowing for the wild-type residues as well using pGV3281 as template and primers #19-40. In addition, the S53D mutation was tested in the context of the recombination library. However, given previous experiences with this mutation (low expression levels, no switch in cofactor specificity, and loss of activity) the recombination library was constructed first, which only contained the mutations found in Generation 1. This was deemed library 1. Next, another round of SOE PCR was used to introduce S or D at position 53 (library 2). Both libraries were separately ligated and transformed. 1.100 of library 1 and 1,700 of library 2 were screened for improved NADH consumption. Having no NADPH screening data for the 2,800 clones and assuming that a switch in cofactor specificity comes with the likely cost of losing activity on NADH, selected variants with improved activity or activity equal to the parent, as well variants that had lost up to 20% of their activity on NADH were rescreened. In total, 88 variants were rescreened on both cofactors narrowing the number down to 14 shown in Table 13. Five double mutants 1A9, 1C2, 1G2, 2A9, and 1G5 were found. The rest were single mutants. None of the 14 showed a switch in cofactor specificity

TABLE 13
Exemplary Hits in Rescreen of Generation 2
Recombination Library.

| | NADH/ | Improve- | | | | | | |
|---|---|---|---|---|---|---|---|---|
| | NADPH | ment in | | | | | | |
| | activity | NADH | | | | | | |
| | ratio | activity | | | | | | |
| Variant | in screen | [%] | V48 | R49 | K52 | S53 | L85 | I89 |
| Parent | 0.34 | — | — | — | — | — | — | — |
| 1A9 | 0.52 | 71 | — | L | — | — | A | — |
| 1C2 | 0.73 | 64 | A | — | — | — | A | — |
| 1E1 | 0.66 | 70 | — | P | — | — | — | — |
| 1E3 | 0.83 | 103 | — | — | — | — | A | — |
| 1G2 | 0.87 | 77 | L | P | — | — | — | — |
| 1G5 | 0.7 | 70 | — | — | — | — | A | A |
| 1G10 | 0.68 | 82 | — | — | — | — | A | — |
| 1H1 | 0.9 | 86 | — | — | — | — | A | — |
| 2A5 | 0.73 | 72.5 | — | V | — | — | — | — |
| 2A8 | 0.51 | 55 | — | P | — | — | — | — |
| 2A9 | 0.5 | 52.5 | L | P | — | — | — | — |
| 2A10 | 0.52 | 59 | — | V | — | — | — | — |
| 2E5 | 0.71 | 34.5 | L | — | — | — | — | — |
| 2G8 | 0.72 | 80 | — | V | — | — | — | — |

Variants 1A9, 1C2, 1G2, and 1G5 were expressed, purified, and characterized (Table 14). Mutation L85A is noteworthy. In all three cases (1A9, 1C2, and 1G5), the NADPH K_{M} value was either cut in half (1A9) or below 1 μM (1C2 and 1G5) and thus beneath the measurable threshold. These variants also showed the lowest NADH K_{M} values with 99, 115, and 112 μM. Mutation L85A could be conceived as being generally activating.

TABLE 14
Comparison of L. lactis KARI and Variants Thereof

| | | mutations | | | | | | U/mg | | | Kₘ [M] for cofactor | |
|---|---|---|---|---|---|---|---|---|---|---|---|---|
| Gen | Variant (gene) | V48 | R49 | K52 | S53 | L85 | I89 | NADH | NADPH | ratio | NADH | NADPH |
| 0 | LI_KARI-^{his6} (wt) | V | R | K | S | L | I | 0.05 | 0.34 ± 0.05 | 0.15 | 285 ± 30 | 13 ± 1.3 |
| 2 | LI_KARI^{1A9-his6} | | L | | | A | | 0.2 | 0.33 | 0.6 | 99 | 8 |
| | LI_KARI^{1G2-his6} | A | | | | A | | 0.17 | 0.33 | 0.5 | 115 | <1 |
| | LI_KARI^{1G2-his6} | L | P | | | | | 0.21 | 0.34 | 0.6 | 150 | 13 |
| | LI_KARI^{1G5-his6} | | | | | A | A | 0.21 | 0.4 | 0.5 | 112 | <1 |

| Gen | Variant (gene) | k_{cat}^{[s-1]} | | k_{cat}/Kₘ [M⁻¹*s⁻¹] | | |
|---|---|---|---|---|---|---|
| 0 | LI_KARI-^{his6} (wt) | NADH | NADPH | NADH | NADPH | ratio |
| 2 | LI_KARI^{1A9-his6} | 0.1 | 0.8 | 530 | 65,000 | 0.008 |
| | LI_KARI^{1G2-his6} | 0.5 | 0.8 | 5,000 | 102,000 | 0.049 |
| | LI_KARI^{1G2-his6} | 0.4 | 0.8 | 3,700 | >800,000 | <0.005 |
| | LI_KARI^{1G5-his6} | 0.5 | 0.8 | 3,300 | 65,000 | 0.051 |
| | | 0.5 | 1.0 | 4,700 | >990,000 | <0.005 |

None of the variants had mutations at residues K52 and S53, positions that bind the NADPH phosphate with high probability. Based on the results of Generation 1, it was hypothesized that the strong NADPH binding is not due to one residue only, but rather due to a concerted binding via R49, K52, and S53. Disrupting the binding of R49 with a mutation such as R49P would give the opportunity to explore different combinations at the two important sites 52 and 53. Thus, a double NNK library at these two positions was generated using a variant with a mutation at residue R49 as template.

Generation 3: Double NNK Library at Positions K52 and S53:

Given the data presented in Table 14, the choice of the parent for the next round was between 1A9 and 1G2. Both had a mutation at position R49 (L and P); in addition, 1A9 carried the L85A mutation described above; 1G2 had a mutation at position 48 (V48L). Even though the NADH K_{M} value and catalytic efficiency of 1G2 were not as favorable as 1A9's, 1G2 was chosen as parent because, due to the lack of L85A, its NADPH activities were parent-like. Thus, its improvements stem from increased NADH activity only.

A library with 1G2 as parent was generated using primers #41 and 42 and the commercial T7 primers (#1 and 2). Approximately 2,800 individual colonies were screened for both NADH and NADPH consumption. The introduction of two negative charges, K52E and S53D, gives the highest NADH/NADPH ratio in the screen (variant 4H8). However, when the order of the residues is reversed, K52D and S53E (variant 3H5), the protein has potential folding issues. The introduction of one negative charge in combination with L, P, S, or K results in at least four-fold ratio improvements compared to parent 1G2. However, when K52 is mutated to P and S53 is still able to bind phosphate, no beneficial effects on the ratio were observed (variant 2G6).

TABLE 15
NADH/NADPH activity ratio measured
in screen of double NNK library.
The variants in this table are the top
hits (except 2G6) of 1/6^{th} of this library.

| | NADH/ | | |
|---|---|---|---|
| | NADPH | | |
| Variant | in screen | K52 | S53 |
| LI_KARI^{1G-his6} | 0.5 | K | S |
| 4H8 | >10 | E | D |
| 3C7 | 8.2 | L | D |
| 3H5 | 8.2 | D | E |
| 3E9 | 6.1 | P | E |
| 2A4 | 3.7 | K | E |
| 3F9 | 2.2 | S | D |
| 2G6 | 0.5 | P | S |

Four out of eight variants are shown in Table 15 (4H8, 3C7, 3H5, and 3E9). In Table 16, characterization data of the cofactor switched variants is presented. Each is compared to the wild-type L. lactis KARI and variants found in Generation 2. The NADPH K_{M} value of LI_KARI^{3C7-his6} is estimated to be greater than 1000 μM. An NADPH concentration of 500 μM in the cuvette in the spectrometer was capable of being measured. At this concentration, the variant had not reached saturation yet. The other two variants, 3H5 and 3E9, had double peaks in the purification chromatogram, low expression levels, and also very low activity after purification.

TABLE 16
Comparison of properties of LI_KARI^{his6} and Generation 2 and Generation 3 enzyme variants.

| | | mutations | | | | | | U/mg | | | Kₘ [μM] for cofactor | |
|---|---|---|---|---|---|---|---|---|---|---|---|---|
| Gen | Variant (gene) | V48 | R49 | K52 | S53 | L85 | I89 | NADH | NADPH | ratio | NADH | NADPH |
| 0 | LI_KARI-^{his6} (wt) | V | R | K | S | L | I | 0.05 | 0.34 ± 0.05 | 0.15 | 285 ± 30 | 13 ± 1.3 |
| 2 | LI_KARI^{1A9-his6} | | L | | | A | | 0.2 | 0.33 | 0.6 | 99 | 8 |
| | LI_KARI^{1C2-his6} | A | | | | A | | 0.17 | 0.33 | 0.5 | 115 | <1 |
| | LI_KARI^{1G2-his6} | L | P | | | | | 0.21 | 0.34 | 0.6 | 150 | 13 |
| | LI_KARI^{1G5-his6} | | | | | A | A | 0.21 | 0.4 | 0.5 | 112 | <1 |
| 3 | LI_KARI^{4H8-his6} | L | P | E | D | | | 0.14 | 0.024 | 5.8 | 128 ± 9 | 1180 ± 280 |
| | LI_KARI^{3C1-his6} | L | P | L | D | | | 0.16 | 0.003 | 53.3 | 108 ± 9 | >1000 |

| | | k_{cat} [^{s-1}] | | k_{cat}/Kₘ [M⁻¹*s⁻¹] | | |
|---|---|---|---|---|---|---|
| Gen | Variant (gene) | NADH | NADPH | NADH | NADPH | ratio |
| 0 | LI_KARI-^{his6} (wt) | 0.1 | 0.8 | 530 | 65,000 | 0.008 |
| 2 | LI_KARI^{1A9-his6} | 0.5 | 0.8 | 5,000 | 102,000 | 0.049 |
| | LI_KARI^{1C2-his6} | 0.4 | 0.8 | 3,700 | >800.000 | <0.005 |
| | LI_KARI^{1G2-his6} | 0.5 | 0.8 | 3,300 | 65,000 | 0.051 |
| 3 | LI_KARI^{1G5-his6} | 0.5 | 1.0 | 4,700 | >990,000 | <0.005 |
| | LI_KARI^{4H8-his6} | 0.35 | 0.06 | 2,700 | <50 | 54 |
| | LI_KARI^{3C1-his6} | 0.4 | 0.01 | 3,700 | <7 | 529 |

Generation 3 variants LI_KARI^{4H8-his6} and LI_KARI^{3C7-his6} exhibit switches in cofactor specificities for NADH over NADPH in terms of catalytic efficiency. Both variants carry four mutations and only differ at position K52 (K52E or K52L, respectively). Residues K52 and S53D appear to be important determinants of cofactor specificity. Both variants have ˜2.5 fold reduced NADH K_{M} values relative to the wild-type L. lactis KARI. Both variants have lost almost all activity (U/mg) on NADPH: 14-fold decrease of activity for 4H8 and 113-fold decrease of activity for 3C7.

In addition to the above-described modifications, further generations of mutants were constructed. Briefly, a recombination library was constructed using standard overlap extension polymerase chain reaction and primer pairs 1, 2, and 43-54. In these additional generations, two variants exhibited beneficial properties: LI_NKR^{Gen6a-his6} and LI_NKR^{Gen6b-his6}. LI_NKR^{Gen6a-his} contained mutations K118E, T182S, and E320K and showed a 3.7-fold increase in catalytic efficiency in the presence of NADH and a 15-fold decrease in 2S-AL K_{M} value. LI_NKR^{Gen6b-his} retained mutations E59K, T182S, and E320K and had an almost 40-fold improved K_{M} value for 2S-AL. The catalytic efficiency of this enzyme in the presence of NADH was 14.8-fold increased compared to its parent, LI_NKR^{Gen3-his6}. LI_NKR^{Gen6b-his} showed a complete switch of cofactor preference. The characterization data is summarized in Table 17.

TABLE 17
Characterization of recombination variants LI_NKR^{Gen6a-his} and LI_NKR^{Gen6b-his} in comparison to their lineage.

| | | | | | | | | | | | | | Kₘ [μM] for | |
|---|---|---|---|---|---|---|---|---|---|---|---|---|---|---|
| | | Mutations | | | | | | | | U/mg | | | cofactor | |
| Gen | Variant (gene) | V48 | R49 | K52 | S53 | E59 | K118 | T182 | E320 | NADH | NADPH | ratio | NADH | NADPH |
| 0 | LI_KARI^{his6} (wt) | | | | | | | | | 0.5 | 0.34 ± 0.05 | 0.15 | 285 ± 30 | 13 ± 1.3 |
| 3 | LI_NKR^{3C7-his6} | L | P | L | D | | | | | 0.16 ± | 0.03 ± | 5 ± | 108 ± | 1000 ± |
| | | | | | | | | | | 0.002 | 0.004 | 0.7 | 9 | 100 |
| 6 | LI_NKR^{Gen6a-his6} | L | P | L | D | | E | S | K | 0.25 ± | 0.16 ± | 1.56 ± | 45 ± | 1059 ± |
| | | | | | | | | | | 0.003 | 0.007 | 0.1 | 19 | 306 |
| 6 | LI_NKR^{Gen6b-his6} | L | P | L | D | K | | S | K | 0.43 ± | 0.14 ± | 3 ± | 15 ± | 749 ± |
| | | | | | | | | | | 0.01 | 0.031 | 0.7 | 4 | 95 |

| | | | | | | | Kₘ | Kᵢ [mM] | | |
|---|---|---|---|---|---|---|---|---|---|---|
| | | | | | | | [mM] for | for R- | | IC₅₀ |
| Gen | Variant (gene) | k_{cat}[s⁻¹] | | k_{cat}/Kₘ[ M⁻¹*s⁻¹] | | | substrate | DHIV | Kᵢ/K_{M} | (mM) for |
| 0 | LI_KARI^{his6} (wt) | NADH | NADPH | NADH | NADPH | ratio | NADH | NADH | NADH | R-DHIV |
| 3 | LI_NKR^{3C7-his6} | 0.1 | 0.8 ± | 430 ± | 65,000 ± | 0.007 ± | 5.6 ± | 1.7 | 0.3 | N.D |
| | | | 0.12 | 45 | 11559 | 0.0 | 1.6 | | | |
| 6 | LI_NKR^{Gen6a-his6} | 0.4 ± | 0.08 ± | 3,704 ± | 80 ± | 46 ± | 8.2 ± | N.D | N.D | N.D |
| | | 0.005 | 0.01 | 312 | 13 | 9 | 1.0 | | | |
| 6 | LI_NKR^{Gen6b-his6} | 0.62 ± | 0.4 ± | 13,808 ± | 375 ± | 37 ± | 0.53 ± | 0.05 ± | 0.1 | 1.05 ± |
| | | 0.01 | 0.02 | 5,834 | 110 | 19 | 0.11 | 0.011 | | 0.26 |
| | | 1.07 ± | 0.35 ± | 71,248 ± | 465 ± | 153 ± | 0.21 ± | 0.01 ± | 0.05 | 0.26 ± |
| | | 0.03 | 0.08 | 19,102 | 122 | 57 | 0.03 | 0.001 | | 0.06 |

### Example 4

### NADH-Dependent KARI Derived from Shewanella and Salmonella

The following example illustrates exemplary long-form KARI enzymes from Shewanella sp. and Salmonella enterica and corresponding NADH-dependent ketol-acid reductoisomerases (NKR) derived therefrom.

Plasmids and primers disclosed in this example are shown in Tables 18-19 below.

TABLE 18
Plasmids Disclosed in Example 4.

| Plasmids | Genotype |
|---|---|
| pET22b(+) | PT7, bla, ori pBR322, lacI, C-term 6xHis |
| pGV3195 | PT7::Se1_KARI^{his6}, bla, oripBR322, lacI |
| pGV3627 | PT7::Sh_sp_KARI_coSc^{his6}, bla, oripBR322, lacI |
| pGV3628 | PT7::Sh_sp_NKR_coSc^{DDhis6}, bla, oripBR322, lacI |
| pGVSh_sp_S78D | PT7::Sh_sp_KARI_coSc^{S78Dhis6}, bla, oripBR322, lacI |
| pGV3629 | PT7::Sh_sp_NKR_coSc^{6E6his6}, bla, oripBR322, lacI |
| pGV3630 | PT7::Se2_KARI_coSc^{his6}, bla, oripBR322, lacI |
| pGVSe2_S78D | PT7::Se2_KARI_coSc^{S78Dhis6}, bla, oripBR322, lacI |
| pGV3631 | PT7::Se2_NKR_coSc^{DDhis6}, bla, oripBR322, lacI |
| pGV3632 | PT7::Se2_NKR_coSc^{6E6his6}, bla, oripBR322, lacI |

TABLE 19
Oligonucleotide Primers Disclosed in Example 4.

| Primer name | Sequence |
|---|---|
| Sh_S78D_for | GCACAAAAGAGAGCCGATTGGCAAAAAGCGAC |
| | (SEQ ID NO: 145) |
| Sh_S78D_rev | GTCGCTTTTTGCCAATCGGCTCTCTTTTGTGC |
| | (SEQ ID NO: 146) |
| Se2_S78D_for | GCAGAAAAGAGAGCCGATTGGCGTAAAGCGACGGA |
| | (SEQ ID NO: 147) |
| Se2_S78D_rev | TCCGTCGCTTTACGCCAATCGGCTCTCTTTTCTGC |
| | (SEQ ID NO: 148) |

Mutations relative to wild-type Salmonella enterica KARI (Se2_KARI) and Shewanella sp. KARI (Sh_sp_KARI) are listed in Table 20 below.

TABLE 20
Mutations Relative to Se2_KARI and Sh_KARI.

| Source | Variant (enzyme) | Mutations |
|---|---|---|
| Salmenella | Se2_KARI | n/a |
| enterica | Se2_KARI^{S78D} | S78D |
| | Se2_NKR^{DD} | R76D, S78D |
| | Se2_NKR^{6E6} | A71S, R76D, |
| | | S78D, Q110V |
| Shewanella sp. | Sh_sp_KARI | n/a |
| | Sh_sp_NKR^{S78D} | S78D |
| | Sh_sp_NKR^{DD} | R76D, S78D |
| | Sh_sp_NKR^{6E6} | A71S, R76D, |
| | | S78D, Q110V |

Genes encoding Se2_KARI, Se2_NKR^{DD}, Se2_NKR^{6E6}, Sh_sp_KARI, Sh_sp_NKR^{DD}, and Sh_sp_NKR^{6E6} were synthesized by GenScript USA Inc. (Piscataway, N.J. 08854 USA) with flanking NdeI and XhoI sites. The genes were isolated by restriction enzyme digestion with NdeI and XhoI for 1 hour at 37° C. The expression vector, pGV3195, was also digested with NdeI and XhoI for 1 hour at 37° C. The fragments were ligated using T4 DNA ligase from New England Biolabs (Ipswich, Mass. USA). The ligated DNAs were transformed into chemically competent E. coli DH5α cells, incubated for 1 h at 37° C. in SOC medium, and plated to LBₐₘₚ agar plates (Luria Bertani Broth, Research Products International Corp, supplemented with 100 μg/mL ampicillin) to yield single colonies. After confirming the correct sequence, E. coli BL21(DE3) cells were transformed with the correct plasmids for expression.

Genes encoding Se2_KARI^{S78D} and Sh_sp_NKR^{S78D}: Single aspartic acid substitutions were introduced using the QuikChange site-directed mutagenesis kit according to manufacturer's protocol (Stratagene). Plasmids pGV3627 and pGV3630 encoding Sh_sp_KARI and Se2_KARI, respectively, were used as templates. The respective primer pairs were primers Sh_S78D_for and Sh_S78D_rev and primers Se2_S78D_for and Se2_S78D_rev. Pfu turbo polymerase (Stratagene) was used as the polymerase in the following PCR program: 95° C. for 2 min; 95° C. for 30 s, 55° C. for 30 s, 72° C. for 8 min (repeat 15 times); 72° C. for 10 min. After the PCR program was completed, the reaction mixtures were digested with DpnI for 1 h at 37° C. Then, chemically competent E. coli XL1-Gold cells were transformed with 3 μL of the un-cleaned PCR mixtures and the cells were allowed to recover in SOC medium at 37° C. with shaking at 250 rpm for 1 h. The recovery allowed the cells to close the nick-containing DNA produced during the PCR and thus to generate circularized plasmids. We then plated varying volumes on LBₐₘₚ agar plates (Luria Bertani Broth, Research Products International Corp, supplemented with 100 μg/mL ampicillin) to yield single colonies. After confirming the correct sequence, E. coli BL21(DE3) cells were transformed with the correct plasmids for expression.

Heterologous expression of Se2_KARI and Sh_sp_KARI variants in E. coli: The expression of Se2_KARI, Sh_sp_KARI, and their corresponding NKR variants (Table 20) was conducted in 0.5 L Erlenmeyer flasks filled with 0.2 L LBₐₘₚ (Luria Bertani Broth, Research Products International Corp, supplemented with 100 μg/mL ampicillin) inoculated with overnight culture to an initial OD₆₀₀ of 0.1. After growing the expression cultures at 37° C. with shaking at 250 rpm for 4 h, the cultivation temperature was dropped to 25° C., and KARI expression was induced with IPTG to a final concentration of 0.5 mM. After 24 h at 25° C. and shaking at 250 rpm, the cells were pelleted at 5,300 g for 10 min and then frozen at −20° C. until further use.

Sh_sp_KARI and its NKR variants were expressed in 200-mL cultures and purified over a 1-mL histrap HP column. The K_{M}, k_{cat}, and specific activity values were measured as described above, and the results are summarized in Table 21. In terms of the ratio of catalytic efficiency with NADH over NADPH, variants Sh_sp_NKR^{S78D}, Sh_sp_NKR^{R76DS78D}, and Sh_sp_NKR^{6E6} can be defined as being NADH-dependent KARIs (NKR) in terms of their catalytic efficiencies.

Se2_KARI and its NKR variants were expressed in 200-mL cultures and purified over a 1-mL histrap HP column. The K_{M}, k_{cat}, and specific activity values were measured as described above, and the results are summarized in Table 22. In terms of the ratio of catalytic efficiency with NADH over NADPH, variants Se2_NKR^{DD} and Se2_NKR^{6E6} can be defined as being NADH-dependent KARIs (NKR). Additional mutations at positions D146, G185, and K433 are generally expected to further improve activity of the Se2_KARI (data not shown).

TABLE 21

| | Comparison of properties of wild-type Shewanella sp. KARI (Sh_sp_KARI), the single and double aspartic acid variants, | | | | | | | | | | | | | |
|---|---|---|---|---|---|---|---|---|---|---|---|---|---|---|
| | and the “6E6” variant. Data is based on measurements using purified proteins. | | | | | | | | | | | | | |
| | Sp. Activity [U/mg] | | | | | | Kₘ [μM] for cofactor | | | | k_{cat}[^{s-1}] | | | |
| | NADH | ± | NADPH | ± | ratio | ± | NADH | ± | NADPH | ± | NADH | ± | NADPH | ± |
| Sh_sp_KARI | 0.30 | 0.012 | 1.2 | 0.013 | 0.2 | 0.0 | 415 | 44 | ≦1 | | 1.1 | 0.05 | 4.5 | 0.05 |
| Sh_sp_NKR^{S78D} | 0.36 | 0.024 | 0.5 | 0.042 | 0.7 | 0.1 | 130 | 15 | 267 | 26 | 1.3 | 0.09 | 2.0 | 0.16 |
| Sh_sp_NKR^{DD} | 0.34 | 0.001 | 0.03 | 0.004 | 10.7 | 1.5 | 90 | 24 | >1000 | | 1.3 | 0.01 | 0.1 | 0.02 |
| Sh_sp_NKR^{6E6} | 0.66 | 0.00 | 0.1 | 0.013 | 9.1 | 1.7 | 75 | 10 | 600 | 130 | 2.4 | 0.00 | 0.3 | 0.05 |

| | k_{cat}/Kₘ[M⁻¹*s⁻¹] | | | | | |
|---|---|---|---|---|---|---|
| | NADH | ± | NADPH | ± | ratio | ± |
| Sh_sp_KARI | 2,649 | 301 | 4,479,890 | | 0.0006 | |
| Sh_sp_NKR^{S78D} | 10,269 | 1,360 | 7,373 | 924 | 1.4 | 0.3 |
| Sh_sp_NKR^{DD} | 14,022 | 3,740 | <117 | | 119 | |
| Sh_sp_NKR^{6E6} | 32,410 | 4,322 | 446 | 127 | 73 | 22.9 |

TABLE 22
Comparison of properties of wild-type Salmonella enterica KARI (Se2_KARI), the single and double aspartic acid
variants, and the “6E6” variant. Data is based on measurements using purified proteins.

| | Sp. Activity [U/mg] | | | | | | Kₘ [μM] for cofactor | | | | k_{cat}[^{s-1}] | | | |
|---|---|---|---|---|---|---|---|---|---|---|---|---|---|---|
| | NADH | ± | NADPH | ± | ratio | ± | NADH | ± | NADPH | ± | NADH | ± | NADPH | ± |
| Se2_KARI | 0.14 | 0.009 | 1.1 | 0.062 | 0.1 | 0.0 | 157 | 4 | 8 | 2 | 0.51 | 0.03 | 3.96 | 0.23 |
| Se2_NKR^{S78D} | 0.17 | 0.005 | 0.4 | 0.015 | 0.4 | 0.0 | 233 | 43 | 272 | 23 | 0.63 | 0.02 | 1.47 | 0.05 |
| Se2_NKR^{DD} | 0.37 | 0.002 | 0.03 | 0.005 | 12.4 | 1.9 | 121 | 20 | >1000 | | 1.36 | 0.01 | 0.11 | 0.02 |
| Se2_NKR^{6E6} | 0.64 | 0.10 | 0.1 | 0.045 | 6.6 | 3.2 | 24 | 4 | 630 | 291 | 2.33 | 0.35 | 0.36 | 0.01 |

| | k_{cat}/Kₘ[M⁻¹*S⁻¹] | | | | | |
|---|---|---|---|---|---|---|
| | NADH | ± | NADPH | ± | ratio | ± |
| Se2_KARI | 3,229 | 222 | 495,409 | 127,118 | 0.01 | 0.0 |
| Se2_NKR^{S78D} | 2,696 | 503 | 5,402 | 498 | 0.5 | 0.1 |
| Se2_NKR^{DD} | 11,222 | 1,856 | <110 | | >102 | |
| Se2_NKR^{6E6} | 97,284 | 21.857 | 565 | 261 | 172 | 88.5 |

The foregoing detailed description has been given for clearness of understanding only and no unnecessary limitations should be understood there from as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

The disclosures, including the claims, figures and/or drawings, of each and every patent, patent application, and publication cited herein are hereby incorporated herein by reference in their entireties.

1.-72. (canceled)

73. A mutant ketol-acid reductoisomerase (KARI) comprising one or more mutations or modifications at positions corresponding to amino acids selected from the group consisting of: (a) valine 48 of the L. lactis KARI (SEQ ID NO: 10); (b) arginine 49 of the L. lactis KARI (SEQ ID NO: 10); (c) lysine 52 of the L. lactis KARI (SEQ ID NO: 10); (d) serine 53 of the L. lactis KARI (SEQ ID NO: 10); (e) glutamic acid 59 of the L. lactis KARI (SEQ ID NO: 10): (f); leucine 85 of the L. lactis KARI (SEQ ID NO: 10); (g) isoleucine 89 of the L. lactis KARI (SEQ ID NO: 10); (h) lysine 118 of the L. lactis KARI (SEQ ID NO: 10); (i) threonine 182 of the L. lactis KARI (SEQ ID NO: 10); and (j) glutamic acid 320 of the L. lactis KARI (SEQ ID NO: 10).

74. The mutant KARI of claim 73, wherein said valine 48 is replaced with a residue selected from leucine or proline.

75. The mutant KARI of claim 73, wherein said arginine 49 is replaced with a residue selected from valine, leucine, serine, or proline.

76. The mutant KARI of claim 73, wherein said lysine 52 is replaced with a residue selected from leucine, alanine, isoleucine, methionine, phenylalanine, tryptophan, tyrosine, valine, aspartic acid, or glutamic acid.

77. The mutant KARI of claim 73, wherein said serine 53 is replaced with a residue selected from aspartic acid or glutamic acid.

78. The mutant KARI of claim 73, wherein said glutamic acid 59 is replaced with a residue selected from lysine, arginine, or histidine.

79. The mutant KARI of claim 73, wherein said leucine 85 is replaced with a residue selected from threonine or alanine.

80. The mutant KARI of claim 73, wherein said isoleucine 89 is replaced with alanine.

81. The mutant KARI of claim 73, wherein said lysine 118 is replaced with a residue selected from glutamic acid or aspartic acid.

82. The mutant KARI of claim 73, wherein said threonine 182 is replaced with a residue selected from serine, asparagine, or glutamine.

83. The mutant KARI of claim 73, wherein said glutamic acid 320 is replaced with a residue selected from lysine, arginine, or histidine.

84. The mutant KARI of claim 73, wherein said mutant KARI is a modified version of a wild-type KARI, and wherein said wild-type KARI is at least 80% identical to SEQ ID NO: 10.

85.-93. (canceled)

94. A recombinant microorganism comprising at least one nucleic acid molecule encoding a mutant KARI of claim 73.

95. (canceled)

96. An isolated nucleic acid molecule encoding a mutant KARI, wherein said mutant KARI comprises one or more mutations or modifications at positions corresponding to amino acids selected from the group consisting of: (a) valine 48 of the L. lactis KARI (SEQ ID NO: 10); (b) arginine 49 of the L. lactis KARI (SEQ ID NO: 10); (c) lysine 52 of the L. lactis KARI (SEQ ID NO: 10); (d) serine 53 of the L. lactis KARI (SEQ ID NO: 10); (e) glutamic acid 59 of the L. lactis KARI (SEQ ID NO: 10): (f); leucine 85 of the L. lactis KARI (SEQ ID NO: 10); (g) isoleucine 89 of the L. lactis KARI (SEQ ID NO: 10); (h) lysine 118 of the L. lactis KARI (SEQ ID NO: 10); (i) threonine 182 of the L. lactis KARI (SEQ ID NO: 10); and (j) glutamic acid 320 of the L. lactis KARI (SEQ ID NO: 10).

97.-98. (canceled)

99. The recombinant microorganism of claim 94, wherein said recombinant microorganism further expresses exogenous genes encoding an acetolactate synthase, a dihydroxy acid dehydratase, a keto-isovalerate decarboxylase, and an alcohol dehydrogenase, wherein said recombinant microorganism produces isobutanol.

100. (canceled)

101. The recombinant microorganism of claim 94, wherein said recombinant microorganism is a yeast microorganism.

102. A method of producing isobutanol, comprising:
(a) providing a recombinant microorganism of claim 99; and
(b) cultivating the recombinant microorganism in a culture medium containing a feedstock providing a carbon source, until the isobutanol is produced.

103. A method of producing isobutanol, comprising:
(a) providing a recombinant microorganism of claim 101; and
(b) cultivating the recombinant microorganism in a culture medium containing a feedstock providing a carbon source, until the isobutanol is produced.


SEQUENCE LISTING (148 sequences)

SEQ ID NO: 1 - DNA, 1479 bp, Shewanella sp.
atggctaactattttaactctctgaatttacgtcaacaattagaacagcttggccaatgccgttttatggatcgctccgagtttagcgatggctgcaattacatcaaagattggaatattgttattttaggttgtggtgctcagggtcttaaccaaggtctgaacatgcgtgactctgggctgaatattgcctacgccctgcgcccagaagccattgcccagaaacgtgcctcatggcaaaaagccacggacaacggctttaaagtgggcacctttgaagagctgatcccaacggcggatttggtactgaacttaacgcccgataagcagcactccaatgtggttagcgctgtgatgccactgatgaaacaaggcgcaacgctgtcttattcccacggttttaacatcgttgaagaaggcatgcagatccgccccgatattacagttgtgatggtagcgcctaagtgcccaggtactgaagtgcgtgaagaatacaagcgtggttttggtgtaccgacactgattgcagtgcacccagaaaacgaccctaatggcgatggtttagagattgccaaagcctatgcgagtgccacaggcggtgaccgcgctggcgtgttgcaatcttcctttattgccgaagtaaaatcggatctgatgggcgaacaaaccattctgtgcggtatgttgcagacgggcgctatcctaggttacgacaagatggtggccgatggtgttgaacctggctatgccgctaagttaatccaacaaggttgggaaaccgtgaccgaggcgcttaagcacggcggtatcaccaacatgatggacagactgtctaatcctgccaagattaaagcgtttgaaattgcagaagatttaaaagaaattctgcaacccttattcgaaaaacatatggatgacatcatcagcggcgagttctcacgcactatgatgcaagactgggcgaatgacgacgctaacctgctgcgttggcgcgccgaaaccgccgaaacgggcttcgaaaatgcccctgtttcgagcgagcatatcgacgagcaaacctatttcgacaaggggattttcctagttgcgatgatcaaagccggtgtcgaattagccttcgatactatggtgtcggcgggtattgttgaagagtcagcctattacgaatcactgcatgaaacgccgctgatcgctaacaccatcgcccgtaaacgtctttacgagatgaacgtggtgatctcggataccgcagaatacggttgttatctgttcaaccatgctgcagtacctatgctgcgtgactatgtaaatgccatgtcgccagagtatttaggtgcgggtctgaaggacagttctaacaacgtcgataacctgcagttaatcgccatcaatgatgcgattcgccacacttcagttgagtatatcggtgcggaacttcgtggttatatgactgatatgaaaagtattgttggagcttaa

SEQ ID NO: 2 - protein, 492 aa, Shewanella sp.
MANYFNSLNLRQQLEQLGQCRFMDRSEFSDGCNYIKDWNIVILGCGAQGLNQGLNMRDSGLNIAYALRPEAIAQKRASWQKATDNGFKVGTFEELIPTADLVLNLTPDKQHSNVVSAVMPLMKQGATLSYSHGFNIVEEGMQIRPDITVVMVAPKCPGTEVREEYKRGFGVPTLIAVHPENDPNGDGLEIAKAYASATGGDRAGVLQSSFIAEVKSDLMGEQTILCGMLQTGAILGYDKMVADGVEPGYAAKLIQQGWETVTEALKHGGITNMMDRLSNPAKIKAFEIAEDLKEILQPLFEKHMDDIISGEFSRTMMQDWANDDANLLRWRAETAETGFENAPVSSEHIDEQTYFDKGIFLVAMIKAGVELAFDTMVSAGIVEESAYYESLHETPLIANTIARKRLYEMNVVISDTAEYGCYLFNHAAVPMLRDYVNAMSPEYLGAGLKDSSNNVDNLQLIAINDAIRHTSVEYIGAELRGYMTDMKSIVGA

SEQ ID NO: 3 - DNA, 1485 bp, Vibrio fischeri
atgtctaactactttaatacgctaaatttacgtgaacaattagatcaactaggtcgttgtcgctttatggatcgtgaagaatttgcaacagaagctgattaccttaaaggtaaaaaagtggttattgttggttgtggtgctcaaggcttaaaccaaggccttaatatgcgtgattcaggcttagatgttgcttatgcactgcgtcaagccgctattgatgagcaacgacaatcttataaaaatgcaaaagaaaatggttttgaagtagctagctatgaaactctgatccctcaagctgacctagttattaatcttactcctgataaacaacatactaatgtagttgaaactgttatgcctctaatgaaagagggggcagctttaggctattcacatggttttaatgttgttgaagaagggatgcaaatccgtaaagatttgacggttgttatggttgctcctaagtgtccaggaacagaagttcgtgaagaatataaacgtggttttggggttccaactctaattgctgttcacccagaaaatgatcctaaaggtgaaggttgggatattgctaaggcttgggctgctggtacaggtggtcaccgtgcgggttgtctagagtcttcttttgtcgctgaagttaaatctgaccttatgggtgagcaaacgattctttgtggcatgctacaagctggttctattgtatcttacgagaagatgattgctgacggtattgagcctggttatgcaggtaaacttctacagtacggttgggaaacaattactgaagccctaaagtttggcggtgttacgcatatgatggatcgcctttcaaacccagctaaagtaaaagcttttgagctttcagaagaacttaaagagctaatgcgtccactttataataagcatatggacgatattatttctggtgagttttctcgcacaatgatggctgattgggctaatgatgatgttaatctatttggctggcgtgaagaaacaggtcaaactgcgtttgaaaactaccctgagtctgatgtagagatctctgaacaagaatactttgataacggtattttactcgttgcaatggttcgtgccggcgttgaattagcttttgaagctatgactgcatcaggcattatcgatgaatcagcttactatgagtcgctgcacgaattaccactgattgctaacactgtagcacgtaagcgtttgtacgaaatgaacgtagttatttctgatactgctgaatatggtaactacctatttgcaaatgtagcaacgccacttcttcgtgagaaattcatgccttctgttgaaacagatgttattggccgaggattaggtgaggcatcaaatcaagtggataatgcaacgctaatcgctgttaatgatgcgattcgtaatcatccagttgaatatattggtgaagaattacgtagctacatgagcgatatgaagcgaattgcagttggtggctaa

SEQ ID NO: 4 - protein, 494 aa, Vibrio fischeri
MSNYFNTLNLREQLDQLGRCRFMDREEFATEADYLKGKKVVIVGCGAQGLNQGLNMRDSGLDVAYALRQAAIDEQRQSYKNAKENGFEVASYETLIPQADLVINLTPDKQHTNVVETVMPLMKEGAALGYSHGFNVVEEGMQIRKDLTVVMVAPKCPGTEVREEYKRGFGVPTLIAVHPENDPKGEGWDIAKAWAAGTGGHRAGCLESSFVAEVKSDLMGEQTILCGMLQAGSIVSYEKMIADGIEPGYAGKLLQYGWETITEALKFGGVTHMMDRLSNPAKVKAFELSEELKELMRPLYNKHMDDIISGEFSRTMMADWANDDVNLFGWREETGQTAFENYPESDVEISEQEYFDNGILLVAMVRAGVELAFEAMTASGIIDESAYYESLHELPLIANTVARKRLYEMNVVISDTAEYGNYLFANVATPLLREKFMPSVETDVIGRGLGEASNQVDNATLIAVNDAIRNHPVEYIGEELRSYMSDMKRIAVGG

SEQ ID NO: 5 - DNA, 1476 bp, Gramella forsetii
atgaccaactattttaacagcctttctttacgtgatcaattagctcagcttggaacctgcaggtttatggagctggatgaattcagcaacgaggtggctgtcctaaaagataaaaaaattgtgatcgtaggctgtggagcccagggtcttaatcaggggctcaatatgcgcgatagcggactcgatatctcatatgcgttaagggaaggagcgattaaagaaaagcgacagtcctggaaaaatgctacggaaaataattttaacgtaggaacttatgaggagcttattccaaaggctgatcttgttatcaatcttacgccagataaacaacatacttcggtgatcaaggcgattcaacctcatatcaaaaaagatgcggtactttcttactctcatggtttcaacattgtggaagaaggaacgaagatacgtgaagatataacggtaattatggtcgcgccaaaatgtcccggaactgaggtgagggaagaatataaaagaggttttggagtgccgactcttatcgcggttcatccggaaaatgatcctcatggaattggcctggattgggcaaaagcttatgcgtatgctacaggtggtcacagggccggagtactggaatcttcttttgttgctgaagtaaaatctgacctaatgggggaacaaacaatgctttgtggagttcttcaaacaggatcgatcttaactttcgataaaatggttgcagatggtgtggagccaaattatgctgcaaaacttatccagtatggatgggaaacaattactgaagccctgaaacatggtggaataaccaatatgatggacaggctttcaaatcctgcaaagcttagagcgaatgaaattgctgaagaacttaaagagaaaatgcgtccgctttttcagaaacatatggatgatataatttcaggagaattcagcagtcgaatgatgcgtgactgggcaaatgatgataaagaattactcacctggcgtgccgaaacagagaataccgcttttgaaaaaactgaagccacttcagaagagatcaaagagcaggaatattttgataaaggtgtgctgatggtggcctttgtaagggcaggtgtagagctggcctttgaaacgatggtggaagccgggataattgaagaatcggcttattatgaatcacttcatgaaactccgcttatagccaataccattgccagaaagaaattatacgagatgaatcgtgtgatttcagatactgctgaatacggttgttatttatttgatcatgctgcaaaaccattggtgaaagattatgtaaactcacttgaaccggaagttgccgggaagaaatttggaacagattgtaatggtgtggataaccagaaattgatacacgtgaatgatgatcttagaagtcatccggttgaaaaagttggagcgagattaagaactgcaatgaccgcaatgaagaaaatatacgcataa

SEQ ID NO: 6 - protein, 491 aa, Gramella forsetii
MTNYFNSLSLRDQLAQLGTCRFMELDEFSNEVAVLKDKKIVIVGCGAQGLNQGLNMRDSGLDISYALREGAIKEKRQSWKNATENNFNVGTYEELIPKADLVINLTPDKQHTSVIKAIQPHIKKDAVLSYSHGFNIVEEGTKIREDITVIMVAPKCPGTEVREEYKRGFGVPTLIAVHPENDPHGIGLDWAKAYAYATGGHRAGVLESSFVAEVKSDLMGEQTMLCGVLQTGSILTFDKMVADGVEPNYAAKLIQYGWETITEALKHGGITNMMDRLSNPAKLRANEIAEELKEKMRPLFQKHMDDIISGEFSSRMMRDWANDDKELLTWRAETENTAFEKTEATSEEIKEQEYFDKGVLMVAFVRAGVELAFETMVEAGIIEESAYYESLHETPLIANTIARKKLYEMNRVISDTAEYGCYLFDHAAKPLVKDYVNSLEPEVAGKKFGTDCNGVDNQKLIHVNDDLRSHPVEKVGARLRTAMTAMKKIYA

SEQ ID NO: 7 - DNA, 1479 bp, Cytophaga hutchinsonii
atggcaaattatttcaatactctttcattaagagaaaaattagatcagttaggcgtttgcgaattcatggacagaagtgagttttctgacggtgtagctgctttgaaaggtaaaaaaattgtaatcgtaggttgtggtgcacaaggtttgaaccagggtttaaaccttcgtgattctggtttagatgtttcttatacattacgtaaagaagccattgattctaaaagacaatcatttttaaatgcttctgaaaatggtttcaaagtgggcacgtacgaagaattaattcctactgctgatttagtaattaacttaacgccggataaacaacatactgctgttgtgtctgcagttatgccattaatgaaaaaaggttctaccttatcttactctcacggtttcaacatcgttgaagaaggtatgcagatccgtaaggacatcacggtaatcatggttgctcctaagtctccgggttctgaagttcgtgaagaatataaaagaggtttcggtgtccctacgttgatcgccgttcaccctgaaaacgatcctgaaggtaaaggctgggattatgctaaggcttactgcgtaggtacaggtggtgacagagctggtgtattgaaatcatctttcgttgctgaagtaaaatctgatttaatgggtgagcaaacaatcctttgtggtttgttgcaaactggttctatcctttgcttcgacaaaatggttgaaaaaggcattgataaaggatatgcttctaaattgatccagtacggatgggaagttatcacggaatcattgaaacatggcggtatcagcggtatgatggatcgtctttcaaaccctgctaaaatcaaggcgttccaggtatctgaagaattgaaagatatcatgcgtccattattccgtaagcatcaggatgatatcatcagcggagaattctcccgcatcatgatggaagactgggcgaatggcgataaaaatttattgacatggagagctgcaacaggtgaaactgcatttgaaaaaacgcctgcaggtgacgttaaaattgctgagcaggaatattatgacaatggtttgctgatggttgctatggttcgtgcgggtgttgaactggcattcgaaacaatgactgaatcaggtatcattgatgaatctgcttactacgaatcattacacgaaacaccgcttatcgcgaacacaatcgcgcgtaagaaattattcgaaatgaaccgtgtaatttctgatacagctgaatacggctgctacttattcgatcatgcctgtaagccattattggcgaacttcatgaagacagtagatacagacatcattggtaaaaacttcaacgcgggtaaagataatggtgttgacaaccagatgctgatcgctgtaaatgaagtattacgttctcacccgatcgaaatcgttggtgctgaattacgtgaagcaatgactgaaatgaaagcaatcgtttcttaa

SEQ ID NO: 8 - protein, 492 aa, Cytophaga hutchinsonii
MANYFNTLSLREKLDQLGVCEFMDRSEFSDGVAALKGKKIVIVGCGAQGLNQGLNLRDSGLDVSYTLRKEAIDSKRQSFLNASENGFKVGTYEELIPTADLVINLTPDKQHTAVVSAVMPLMKKGSTLSYSHGFNIVEEGMQIRKDITVIMVAPKSPGSEVREEYKRGFGVPTLIAVHPENDPEGKGWDYAKAYCVGTGGDRAGVLKSSFVAEVKSDLMGEQTILCGLLQTGSILCFDKMVEKGIDKGYASKLIQYGWEVITESLKHGGISGMMDRLSNPAKIKAFQVSEELKDIMRPLFRKHQDDIISGEFSRIMMEDWANGDKNLLTWRAATGETAFEKTPAGDVKIAEQEYYDNGLLMVAMVRAGVELAFETMTESGIIDESAYYESLHETPLIANTIARKKLFEMNRVISDTAEYGCYLFDHACKPLLANFMKTVDTDIIGKNFNAGKDNGVDNQMLIAVNEVLRSHPIEIVGAELREAMTEMKAIVS

SEQ ID NO: 9 - DNA, 1023 bp, Lactococcus lactis
atggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcagtagctaaagctgatgttattatggttttggcaccagatgaacttcaacaatccatttatgaagaggacatcaaaccaaacttgaaagcaggttcagcacttggttttgctcacggatttaatatccattttggctatattaaagtaccagaagacgttgacgtctttatggttgcgcctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacttttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccacggattattactgacgaagttaaaaagaatatgaagcttgttttggctgatattcaatctggaaaatttgctcaagatttcgttgatgacttcaaagcggggcgtccaaaattaatagcctatcgcgaagctgcaaaaaatcttgaaattgaaaaaattggggcagagctacgtcaagcaatgccattcacacaatctggtgatgacgatgcctttaaaatctatcagtaa

SEQ ID NO: 10 - protein, 340 aa, Lactococcus lactis
MAVTMYYEDDVEVSALAGKQIAVIGYGSQGHAHAQNLRDSGHNVIIGVRHGKSFDKAKEDGFETFEVGEAVAKADVIMVLAPDELQQSIYEEDIKPNLKAGSALGFAHGFNIHFGYIKVPEDVDVFMVAPKAPGHLVRRTYTEGFGTPALFVSHQNASGHAREIAMDWAKGIGCARVGIIETTFKEETEEDLFGEQAVLCGGLTALVEAGFETLTEAGYAGELAYFEVLHEMKLIVDLMYEGGFTKMRQSISNTAEFGDYVTGPRIITDEVKKNMKLVLADIQSGKFAQDFVDDFKAGRPKLIAYREAAKNLEIEKIGAELRQAMPFTQSGDDDAFKIYQ

SEQ ID NO: 11 - DNA, 1023 bp, Lactococcus lactis
atggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcggtagctaaagctgatgttattatggttttggcgccagatgaacttcaacaatccatttatgaagaggacatcaaaccaaacttgaaagcaggttcagcacttggttttgctcacggatttaatatccattttggctatattaaagtaccagaagacgttgacgtctttatggttgcacctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacctttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccaaggattattactgacgcagttaaaaagaatatgaagcttgttttggctgatattcaatctggaaaatttgctcaagatttcgttgatgacttcaaagcggggcgtccaaaattaacagcctatcgcgaagctgctaaaaatcttgaaattgaaaaaattggggcagaattacgtaaagcaatgccattcacacaatctggtgatgacgatgcctttaaaatctatcagtaa

SEQ ID NO: 12 - protein, 340 aa, Lactococcus lactis
MAVTMYYEDDVEVSALAGKQIAVIGYGSQGHAHAQNLRDSGHNVIIGVRHGKSFDKAKEDGFETFEVGEAVAKADVIMVLAPDELQQSIYEEDIKPNLKAGSALGFAHGFNIHFGYIKVPEDVDVFMVAPKAPGHLVRRTYTEGFGTPALFVSHQNASGHAREIAMDWAKGIGCARVGIIETTFKEETEEDLFGEQAVLCGGLTALVEAGFETLTEAGYAGELAYFEVLHEMKLIVDLMYEGGFTKMRQSISNTAEFGDYVTGPRIITDAVKKNMKLVLADIQSGKFAQDFVDDFKAGRPKLTAYREAAKNLEIEKIGAELRKAMPFTQSGDDDAFKIYQ

SEQ ID NO: 13 - DNA, 1023 bp, Lactococcus lactis
atggcagttacaatgtattatgaagatgatgtagaagtatcagcacttgctggaaagcaaattgcagtaatcggttatggttcacaaggacatgctcacgcacagaatttgcatgattctggtcacaacgttatcattggtgtgcgccacggaaaatcttttgataaagcaaaagaagatggctttgaaacatttgaagtaggagaagcggtagctaaagctgatgttattatggttttggcgccagatgaacttcaacaatccatttatgaagaggacatcaaaccaaacttgaaagcaggttcagcacttggttttgctcacggatttaatatccattttggctatattaaagtaccagaagacgttgacgtctttatggttgcacctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacctttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccaaggattattactgacgcagttaaaaagaatatgaagcttgttttggctgatattcaatctggaaaatttgctcaagatttcgttgatgacttcaaagcggggcgtccaaaattaacagcctatcgcgaagctgctaaaaatcttgaaattgaaaaaattggggcagaattacgtaaagcaatgccattcacacaatctggtgatgacgatgcctttaaaatctatcagtaa

SEQ ID NO: 14 - protein, 340 aa, Lactococcus lactis
MAVTMYYEDDVEVSALAGKQIAVIGYGSQGHAHAQNLHDSGHNVIIGVRHGKSFDKAKEDGFETFEVGEAVAKADVIMVLAPDELQQSIYEEDIKPNLKAGSALGFAHGFNIHFGYIKVPEDVDVFMVAPKAPGHLVRRTYTEGFGTPALFVSHQNASGHAREIAMDWAKGIGCARVGIIETTFKEETEEDLFGEQAVLCGGLTALVEAGFETLTEAGYAGELAYFEVLHEMKLIVDLMYEGGFTKMRQSISNTAEFGDYVTGPRIITDAVKKNMKLVLADIQSGKFAQDFVDDFKAGRPKLTAYREAAKNLEIEKIGAELRKAMPFTQSGDDDAFKIYQ

SEQ ID NO: 15 - DNA, 1023 bp, Lactococcus lactis
atggcagttacaatgtattatgaagaagatgtagaagtagccgcactcgcgggtaagaaaatcgcagtgattggatatggctcacaaggacacgctcatgcacaaaacttgcgtgattctggtcatgatgtgattattggtgtccgtcaggggaaatcttttgataaagcaaaagaagatggttttgaaacatttgaagtaggagaagcagtagctaaagctgacgtcattatggttctggcacctgatgaacttcaacaatctatttatgaagaggacataaaaccaaatttgaaagcaggttcagcacttggttttgcccatggtttcaatattcattttggctatattgaagttccagaagatgttgatgtcttcatggttgcgccaaaagcgccgggacatctcgttcggcggacttttaccgaaggtttcggaacgccagctttgttcgtttcgcatcaaaatgccactggtcatgcgcgtgaaattgccatggactgggccaaaggaattggctgtgcccgtgtcggtatcattgaaacaactttcaaagaagaaacagaagaagatttgtttggcgaacaggccgtgctttgtggcggtttgacagcacttgttgaagctggttttgaaacactgacagaagctggatatgctggcgaattggcttactttgaagtgctgcatgaaatgaaattgattgttgaccttatgtacgaaggtggtttcactaaaatgcgtcagtcaatctcaaacactgccgaatttggtgattatgtgactggaccacgcattattactgacgaagttaaaaagaatatgaaactcgtgttggctgacattcaatcaggaaaatttgcgcaagatttcgttgatgatttcaaagctggacgtccaaaattaactgcttatcgtgaagcagctaaaaatctggaaattgaaaaaatcggtgcagaactacgtaaagcaatgccatttacacaatctggtgatgacgacgcctttaaaatttatcaataa

SEQ ID NO: 16 - protein, 340 aa, Lactococcus lactis
MAVTMYYEEDVEVAALAGKKIAVIGYGSQGHAHAQNLRDSGHDVIIGVRQGKSFDKAKEDGFETFEVGEAVAKADVIMVLAPDELQQSIYEEDIKPNLKAGSALGFAHGFNIHFGYIEVPEDVDVFMVAPKAPGHLVRRTFTEGFGTPALFVSHQNATGHAREIAMDWAKGIGCARVGIIETTFKEETEEDLFGEQAVLCGGLTALVEAGFETLTEAGYAGELAYFEVLHEMKLIVDLMYEGGFTKMRQSISNTAEFGDYVTGPRIITDEVKKNMKLVLADIQSGKFAQDFVDDFKAGRPKLTAYREAAKNLEIEKIGAELRKAMPFTQSGDDDAFKIYQ

SEQ ID NO: 17 - DNA, 1023 bp, Lactococcus lactis; identical to SEQ ID NO: 15

SEQ ID NO: 18 - protein, 340 aa, Lactococcus lactis; identical to SEQ ID NO: 16

SEQ ID NO: 19 - DNA, 1023 bp, Lactococcus lactis
atggcagttacaatgtattatgaagaagatgtagaagtagccgcactcgcgggtaagaaaatcgcagtgattggatatggctcacaaggacacgctcatgcacaaaacttgcgtgattctggtcatgatgtgattattggcgttcgtcaggggaaatcttttgatagagcaaaagaagatggctttgaaacatttgaagtaggagaagcagtagctaaagctgatgtcattatggttctggcacctgatgaacttcaacaatctatttatgaagaggacataaaaccaaatttgaaatcaggttcagcacttggttttgcccatggtttcaatattcattttggctatattgaagttccagaagatgttgatgtcttcatggttgcgccaaaagcgccgggacatctcgttcggcggacttttaccgaaggtttcggaacgccagctttgttcgtttcgcatcaaaatgccactggtcatgcgcgtgaaatcgctatggactgggcgaaaggcattggttgtgcccgtgtgggaattatcgaaacaactttcaaagaagaaacagaagaagatttgtttggcgaacaagctgtgctttgtggtggtttgacagcacttgttgaagctggttttgaaacactgacagaagctagatatgctggtgaattggcttactttgaagtgctgcatgaaatgaaattgattgttgaccttatgtacgaaggtggtttcactaaaatgcgtcagtcaatctcaaatactgccgaatttggcgattatgtgactggaccacgcattattactgacgaagttaaaaagaatatgaaactcgtgttggctgacattcaatcaggaaaatttgcgcaagatttcgttgatgatttcaaagctggacgtccaaaattaactgcttatcgtgaagcagctaaaaatctggaaattgaaaaaatcggtgcagaactacgtaaagcaatgccatttacacaatctggtgatgacgacgcctttaaaatttatcaataa

SEQ ID NO: 20 - protein, 340 aa, Lactococcus lactis
MAVTMYYEEDVEVAALAGKKIAVIGYGSQGHAHAQNLRDSGHDVIIGVRQGKSFDRAKEDGFETFEVGEAVAKADVIMVLAPDELQQSIYEEDIKPNLKSGSALGFAHGFNIHFGYIEVPEDVDVFMVAPKAPGHLVRRTFTEGFGTPALFVSHQNATGHAREIAMDWAKGIGCARVGIIETTFKEETEEDLFGEQAVLCGGLTALVEAGFETLTEARYAGELAYFEVLHEMKLIVDLMYEGGFTKMRQSISNTAEFGDYVTGPRIITDEVKKNMKLVLADIQSGKFAQDFVDDFKAGRPKLTAYREAAKNLEIEKIGAELRKAMPFTQSGDDDAFKIYQ

SEQ ID NO: 21 - DNA, 1034 bp, Lactococcus lactis
atggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcagtagctaaagctgatgttattatggttttggcaccagatgaacttcaacaatccatttatgaagaggacatcaaaccaaacttgaaagcaggttcagcacttggttttgctcacggatttaatatccattttggctatattaaagtaccagaagacgttgacgtctttatggttgcgcctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacttttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccacggattattactgacgaagttaaaaagaatatgaagcttgttttggctgatattcaatctggaaaatttgctcaagatttcgttgatgacttcaaagcggggcgtccaaaattaatagcctatcgcgaagctgcaaaaaatcttgaaattgaaaaaattggggcagagcacgtcaagcaatgccattcacacaatctggtgatgacgatgcctttaaaatctatcagtaatttctcttattg

SEQ ID NO: 22 - protein, 344 aa, Lactococcus lactis
MAVTMYYEDDVEVSALAGKQIAVIGYGSQGHAHAQNLRDSGHNVIIGVRHGKSFDKAKEDGFETFEVGEAVAKADVIMVLAPDELQQSIYEEDIKPNLKAGSALGFAHGFNIHFGYIKVPEDVDVFMVAPKAPGHLVRRTYTEGFGTPALFVSHQNASGHAREIAMDWAKGIGCARVGIIETTFKEETEEDLFGEQAVLCGGLTALVEAGFETLTEAGYAGELAYFEVLHEMKLIVDLMYEGGFTKMRQSISNTAEFGDYVTGPRIITDEVKKNMKLVLADIQSGKFAQDFVDDFKAGRPKLIAYREAAKNLEIEKIGAEHVKQCHSHNLVMTMPLKSISNFSY

SEQ ID NO: 23 - DNA, 1023 bp, Streptococcus equinus
atggcagtaacaatggaatacgaaaaagatgtaaaagtagcagctcttgatggtaaaaaaattgccgttatcggttatggatcacaaggtcatgctcatgctcaaaacttacgtgattcaggtcacgatgttatcattggggttcgccatggtaaatcattcgacaaagcaaaagaagatggtttcgaaacatatgaagtagctgaagcaacaaaacttgctgatgttatcatggttttggcaccagatgaaatccaagcaaaactttatgctgaagaaattgcaccaaatcttgaagcaggtaatgcacttggttttgctcatggtttcaatatccgttttgaatatattaaagctccagaaacagtagatgtctttatgtgtgcgcctaaaggtccaggtcaccttgtacgccgtacttacactgaaggatttggtgtgccagcactttacgctgtttatcaagatgctactggtcatgctaaagacatcgcaatggactggtctaaaggtatcggtgctgcgcgtgttggacttcttgaaactacattcaaagaagaaactgaagaagatttgttcggtgaacaagcagttctttgtggtggtttgactgcccttatcgaagcaggttttgaagttcttactgaagcaggctatgctccagaattggcttacttcgaagttcttcatgaaatgaaacttatcgttgaccttatttacgaaggtggattcaagaaaatgcgtcaatcaatttcaaatacagctgaatttggtgactatgtttcaggtccacgtgtcatcactaaagatgttaaagaaaatatgaaagccgttcttgctgatattcaatcaggtaaatttgctgaagaatttgtaagcgactataaagctggtcgtccaaaacttgaagcttatcgtaaagaagctgcagaacttgaaattgaaaaagtgggtgcagaacttcgtaaagcaatgccttttgttaaccaaaatgatgacgatgcattcaaaatttataactaa

SEQ ID NO: 24 - protein, 340 aa, Streptococcus equinus
MAVTMEYEKDVKVAALDGKKIAVIGYGSQGHAHAQNLRDSGHDVIIGVRHGKSFDKAKEDGFETYEVAEATKLADVIMVLAPDEIQAKLYAEEIAPNLEAGNALGFAHGFNIRFEYIKAPETVDVFMCAPKGPGHLVRRTYTEGFGVPALYAVYQDATGHAKDIAMDWSKGIGAARVGLLETTFKEETEEDLFGEQAVLCGGLTALIEAGFEVLTEAGYAPELAYFEVLHEMKLIVDLIYEGGFKKMRQSISNTAEFGDYVSGPRVITKDVKENMKAVLADIQSGKFAEEFVSDYKAGRPKLEAYRKEAAELEIEKVGAELRKAMPFVNQNDDDAFKIYN

SEQ ID NO: 25 - DNA, 1023 bp, Streptococcus infantarius
atggcagtaacaatggaatacgaaaaagacgtaaaagtagcagctcttgatggtaaaaaaattgccgttattggttatggatcacaaggtcatgctcatgctcaaaacttgcgtgactcaggtcacgatgttatcattggggttcgccatggtaaatcattcgataaagcaaaagaagatggatttgatacttatgaagtagcagaagcaacaaaacttgctgatgttatcatggtattggctcctgatgaaatccaagctaaactttatgctgaagaaatcgctccaaaccttgaagctggtaacgctcttggatttgcacatggttttaatatccgttttggatacattaaagctccagaaacagtagatgtcttcatgtgtgctcctaaaggaccaggtcaccttgttcgtcgtacttacacagaaggatttggtgtaccagcactttacgctgtttaccaagatgctactggtaatgctaaagacatcgcaatggattggtctaaaggtatcggtgctgcacgtgttggacttcttgaaacaacatttaaagaagaaactgaagaagacctctttggtgaacaagcagtactttgtggtggtttaactgctcttatcgaagctggttttgaagttcttactgaagctggctatgctccagaattggcttactttgaagttcttcatgaaatgaaacttatcgttgaccttatctacgaaggtggattcaagaaaatgcgtcaatcaatttcaaatacagctgaatttggtgactacgtatctggaccacgtgttatcactaaagatgttaaagaaaatatgaaagctgttcttgctgatatccaatcaggtaaattcgctgaagattttgttaacgactaccaagcaggtcgtccaaaacttgaagcataccgtaaagaagctgcagctcttgaaattgaaaaagtgggtgctgaacttcgtaaagcaatgccttttgttaaccaaaacgatgacgatgcattcaaaatttataactaa

SEQ ID NO: 26 - protein, 340 aa, Streptococcus infantarius
MAVTMEYEKDVKVAALDGKKIAVIGYGSQGHAHAQNLRDSGHDVIIGVRHGKSFDKAKEDGFDTYEVAEATKLADVIMVLAPDEIQAKLYAEEIAPNLEAGNALGFAHGFNIRFGYIKAPETVDVFMCAPKGPGHLVRRTYTEGFGVPALYAVYQDATGNAKDIAMDWSKGIGAARVGLLETTFKEETEEDLFGEQAVLCGGLTALIEAGFEVLTEAGYAPELAYFEVLHEMKLIVDLIYEGGFKKMRQSISNTAEFGDYVSGPRVITKDVKENMKAVLADIQSGKFAEDFVNDYQAGRPKLEAYRKEAAALEIEKVGAELRKAMPFVNQNDDDAFKIYN

SEQ ID NO: 27 - DNA, 993 bp, Methanococcus maripaludis
atgaaggtattctatgactcagattttaaattagatgctttaaaagaaaaaacaattgcagtaatcggttatggaagtcaaggtagggcacagtccttaaacatgaaagacagcggattaaacgttgttgttggtttaagaaaaaacggtgcttcatgggaaaacgctaaagcagacggtcacaacgtaatgactatcgaagaagctgctgaaaaagctgacatcatccacatcttaattcctgacgaattacaggcagaagtttatgaaagccagataaaaccatatttaaaggaaggaaaaacactcagcttttcacatggttttaacatccactatggattcattgttccaccaaagggagttaacgtggttttagttgctccaaaatcacctggaaaaatggttagaagaacatacgaagaaggcttcggtgttccaggtttaatctgtatcgaaatcgatgcaacaaacaacgcatttgacattgtttcagcaatggcaaaaggaatcggtttatcaagggccggagttatccagacaactttcaaagaagaaacagaaactgaccttttcggtgaacaagctgttttatgcggcggagttaccgaattaatcaaagcaggattcgaaacacttgttgaagcaggatacgcaccagaaatggcatactttgaaacatgccacgaattgaaattaattgttgacttaatctaccaaaaaggattcaaaaacatgtggaacgatgtaagtaacactgcagaatacggtggacttacaagaagaagcagaatcgttacagctgactcaaaagctgcaatgaaagaaatcttaaaagaaatccaagatggaagattcacaaaagaattcgtgcttgaaaaacaagtaaaccacgcgcacttaaaagcaatgagaagaatcgaaggagacttacaaatcgaagaagtcggtgcaaaattaagaaaaatgtgcggtcttgaaaaagaagaatga

SEQ ID NO: 28 - protein, 330 aa, Methanococcus maripaludis
MKVFYDSDFKLDALKEKTIAVIGYGSQGRAQSLNMKDSGLNVVVGLRKNGASWENAKADGHNVMTIEEAAEKADIIHILIPDELQAEVYESQIKPYLKEGKTLSFSHGFNIHYGFIVPPKGVNVVLVAPKSPGKMVRRTYEEGFGVPGLICIEIDATNNAFDIVSAMAKGIGLSRAGVIQTTFKEETETDLFGEQAVLCGGVTELIKAGFETLVEAGYAPEMAYFETCHELKLIVDLIYQKGFKNMWNDVSNTAEYGGLTRRSRIVTADSKAAMKEILKEIQDGRFTKEFVLEKQVNHAHLKAMRRIEGDLQIEEVGAKLRKMCGLEKEE

SEQ ID NO: 29 - DNA, 993 bp, Methanococcus maripaludis
atgaaggtattctatgactcagattttaaattagatgctttaaaagaaaaaacaattgcagtaatcggttacggaagtcaaggaagagcacagtccttaaacatgaaagacagtggattaaacgttgttgttggtttaaggaaaaatggagcttcgtgggaaaacgctaaagcagacggtcacaatgtaatgactatcgaagaagctgctgaaaaagctgacatcatccacatcttaatccctgacgaattacaggcagaagtttacgatgctcaaataaaaccatacctcaaagaaggaaaaacactcagtttctcacatggttttaacatccactatggattcattgttccaccaaaaggagttaacgtggttttagttgctccaaaatcacctggaaaaatggttagaagaacatacgaagaaggtttcggtgttccaggtttaatctgtatcgaaatagatgcaacaaacaacgcatttgacattgtttcagcaatggcaaaaggaatcggtttatcaagagctggagttatccagacaacatttaaagaagaaacagaaactgaccttttcggtgaacaagctgttttatgcggtggagttaccgaattaatcaaagcaggatttgaaacactcgtagaagcaggatacgcaccagaaatggcatactttgaaacatgccacgaattgaaattaatcgttgacttaatctaccaaaaaggattcaaaaacatgtggaacgacgtaagtaacactgcagaatacggtggacttacaagaagaagcagaattgtaactgctgattcaaaagctgcaatgaaagaaatcttaaaagaaatccaagacggaagattcacaaaagaattcgttcttgaaaaacaagtaaaccacgcacatttaaaagcaatgagaagactcgaaggagaattacaaatcgaagaagtcggtgcaaaattaagaaaaatgtgcggtcttgaaaaagaagaataa

SEQ ID NO: 30 - protein, 330 aa, Methanococcus maripaludis
MKVFYDSDFKLDALKEKTIAVIGYGSQGRAQSLNMKDSGLNVVVGLRKNGASWENAKADGHNVMTIEEAAEKADIIHILIPDELQAEVYDAQIKPYLKEGKTLSFSHGFNIHYGFIVPPKGVNVVLVAPKSPGKMVRRTYEEGFGVPGLICIEIDATNNAFDIVSAMAKGIGLSRAGVIQTTFKEETETDLFGEQAVLCGGVTELIKAGFETLVEAGYAPEMAYFETCHELKLIVDLIYQKGFKNMWNDVSNTAEYGGLTRRSRIVTADSKAAMKEILKEIQDGRFTKEFVLEKQVNHAHLKAMRRLEGELQIEEVGAKLRKMCGLEKEE

SEQ ID NO: 31 - DNA, 993 bp, Methanococcus maripaludis
atgaaggtattctatgactcagattttaaattagatgctttaaaagaaaaaacaatcgcagtaatcggttacggaagccaaggtcgggcacagtccttaaacatgaaagacagcggattaaacgttgttgttggtttaagaaaaaatggtgcttcatgggaaaacgctaaagcagacggtcacaatgtaatgaccatcgaagaagctgctgaaaaagctgacatcatccacatcttaatccctgacgaattacaggcagaagtttacgatgctcaaataaaaccatgcctcaaagaaggaaaaacactcagcttttcacatggttttaacatccactatggattcattgttccaccaaaaggagttaacgtggttttagttgctccaaaatcaccaggaaaaatggttagaagaacatacgaagaaggtttcggtgttccaggattaatctgtatcgaaattgatgcaacaaacaatgcatttgacattgtttcagcaatggcaaaaggaatcggtttatcaagagctggagttatccagacaacatttaaagaagaaacagaaaccgatcttttcggtgaacaagctgttttatgcggtggagttaccgaattaatcaaagcaggatttgaaacactcgttgaagcaggatacgcaccagaaatggcttactttgaaacatgtcacgaattgaaattaattgttgacttaatctaccaaaaaggattcaaaaacatgtggaacgatgtaagtaacactgctgaatatggtggacttacaagaagaagcagaattgttactgctgactcaaaagctgcaatgaaagaaattttaaaagaaatccaagacggaagattcacaaaagaattcgtgcttgaaaaacaagtaaaccacgcgcacttaaaagcaatgagaagaatcgaaggagaattacaaatcgaggaagtcggcgcaaaattaagaaaaatgtgcggtcttgaaaaagaagaataa

SEQ ID NO: 32 - protein, 330 aa, Methanococcus maripaludis
MKVFYDSDFKLDALKEKTIAVIGYGSQGRAQSLNMKDSGLNVVVGLRKNGASWENAKADGHNVMTIEEAAEKADIIHILIPDELQAEVYDAQIKPCLKEGKTLSFSHGFNIHYGFIVPPKGVNVVLVAPKSPGKMVRRTYEEGFGVPGLICIEIDATNNAFDIVSAMAKGIGLSRAGVIQTTFKEETETDLFGEQAVLCGGVTELIKAGFETLVEAGYAPEMAYFETCHELKLIVDLIYQKGFKNMWNDVSNTAEYGGLTRRSRIVTADSKAAMKEILKEIQDGRFTKEFVLEKQVNHAHLKAMRRIEGELQIEEVGAKLRKMCGLEKEE

SEQ ID NO: 33 - DNA, 993 bp, Methanococcus maripaludis
atgaaggtattctatgactcagattttaaattagatgctttaaaagaaaaaacaattgcagtaatcggttatggaagtcaaggtagggcacagtccttaaacatgaaagacagcggattaaacgttgttgttggtttaagaaaaaacggtgcttcatggaacaacgctaaagcagacggtcacaatgtaatgaccattgaagaagctgctgaaaaagcggacatcatccacatcttaatacctgatgaattacaggcagaagtttatgaaagccagataaaaccatacctaaaagaaggaaaaacactaagcttttcacatggttttaacatccactatggattcattgttccaccaaaaggagttaacgtggttttagttgctccaaaatcacctggaaaaatggttagaagaacatacgaagaaggtttcggtgttccaggtttaatctgtattgaaattgatgcaacaaacaacgcatttgatattgtttcagcaatggcaaaaggaatcggtttatcaagagctggagttatccagacaactttcaaagaagaaacagaaactgaccttttcggtgaacaagctgttttatgcggtggagttaccgaattaatcaaggcaggatttgaaacactcgttgaagcaggatacgcaccagaaatggcatactttgaaacctgccacgaattgaaattaatcgttgacttaatctaccaaaaaggattcaaaaacatgtggaacgatgtaagtaacactgcagaatacggcggacttacaagaagaagcagaatcgttacagctgattcaaaagctgcaatgaaagaaatcttaagagaaatccaagatggaagattcacaaaagaattccttctcgaaaaacaggtaagctatgctcatttaaaatcaatgagaagactcgaaggagacttacaaatcgaagaagtcggcgcaaaattaagaaaaatgtgcggtcttgaaaaagaagaataa

SEQ ID NO: 34 - protein, 330 aa, Methanococcus maripaludis
MKVFYDSDFKLDALKEKTIAVIGYGSQGRAQSLNMKDSGLNVVVGLRKNGASWNNAKADGHNVMTIEEAAEKADIIHILIPDELQAEVYESQIKPYLKEGKTLSFSHGFNIHYGFIVPPKGVNVVLVAPKSPGKMVRRTYEEGFGVPGLICIEIDATNNAFDIVSAMAKGIGLSRAGVIQTTFKEETETDLFGEQAVLCGGVTELIKAGFETLVEAGYAPEMAYFETCHELKLIVDLIYQKGFKNMWNDVSNTAEYGGLTRRSRIVTADSKAAMKEILREIQDGRFTKEFLLEKQVSYAHLKSMRRLEGDLQIEEVGAKLRKMCGLEKEE

SEQ ID NO: 35 - DNA, 993 bp, Methanococcus vannielii
atgaaggtattctacgatgcagacataaaattagacgctttaaaaagtaaaacaattgcagttattggttacggaagtcagggtagagcccagtctttaaacatgaaagacagcggtttaaacgttgtagttggtttaaggaaaaacggtgcttcatgggaaaacgctaaaaacgatggtcatgaagtattaacgattgaagaagcttcaaaaaaagcagacataattcatatattaatccctgatgaattacaggctgaagtttacgaaagccagataaaaccataccttacagaaggaaaaacattaagcttctcacacggctttaatatccattatgggtttattattccgccaaaaggagttaacgtggttttagttgcaccaaagtcacccggtaaaatggttagaaaaacatacgaagaaggatttggtgttccgggattaatctgtatagaagtagatgctacaaatactgcatttgagactgtttcagcaatggcaaaggggatcggcctctcaagagcaggcgttatccagacaacatttagggaggaaactgaaaccgatctttttggtgaacaggcagtattgtgcggcggagttactgaattaattaaagcaggatttgaaacactcgttgaagcaggatattcacctgaaatggcgtattttgaaacatgccacgagttaaaattaattgttgacttaatttaccaaaaaggattcaaaaacatgtggcatgatgtaagtaatactgcagaatatggtggacttacaagaagaagcagaatcgttactgctgactcaaaagctgcgatgaaagaaattttaaaagagattcaagatggaagatttacaaaagaatttgttcttgaaaatcaagctaaaatggcacatttaaaagcaatgaggagacttgaaggcgaattgcaaattgaagaagtcggttcaaagttaagaaaaatgtgtggtcttgaaaaagacgaataa

SEQ ID NO: 36 - protein, 330 aa, Methanococcus vannielii
MKVFYDADIKLDALKSKTIAVIGYGSQGRAQSLNMKDSGLNVVVGLRKNGASWENAKNDGHEVLTIEEASKKADIIHILIPDELQAEVYESQIKPYLTEGKTLSFSHGFNIHYGFIIPPKGVNVVLVAPKSPGKMVRKTYEEGFGVPGLICIEVDATNTAFETVSAMAKGIGLSRAGVIQTTFREETETDLFGEQAVLCGGVTELIKAGFETLVEAGYSPEMAYFETCHELKLIVDLIYQKGFKNMWHDVSNTAEYGGLTRRSRIVTADSKAAMKEILKEIQDGRFTKEFVLENQAKMAHLKAMRRLEGELQIEEVGSKLRKMCGLEKDE

SEQ ID NO: 37 - DNA, 990 bp, Methanococcus voltae
atgcaagtactttacgaagctgacgcaaactatgataaattaaaaggcaaaacaatagcagttatcggatacggtagccaaggtagagctcaatcattaaatatgaaagagagcggtttaaacgtaataatgggtttaagagaaggcggtgcatcctgggaatctgctaaaaaagacggccacgaagtatactcaatcgaggaagctgcaaaaatggctgacgttatacatatattaatacctgacgaaatccaaggtaatgtatacaatagccaaataaaaccttatttggaagaaggcaacacattaagcttttcacacggttataatatccattttaactacattgtagcaccaaaaggtgttaatataacaatggtagctcctaaatcacctggaaaaatggtaagaaaaacctacgaagaaggtttcggtgtacctggtttaatctgcatcgaaaaagacgaaactggcgaagcttacgatattgcattaggtatggcaaaaggtatcggattaacaagagcgggagttattcaaacaacattcagggaagaaacagaaaccgatttattcggtgagcaagctgttctctgtggtggcgttactgaattaatcaaagcaggatttgagacacttgttgaagcaggatatgctccagaaatggcttactttgaaacttgccacgaattgaaattaatcgttgatttaatctaccaaaaaggatttaaaaatatgtggcacgatgtaagtaatactgcggaatatggtggacttaccagaagagaaagagtagttacaaaagaatcaaaagaagcaatgaaggaaatcttaaaagaaatccaagatggaagatttacaaaagaatttgctctcgaaaaccaagctggaaaacctcacttaaattcaatgagaagattagaaggagaattactcatcgaacaagtaggtgctgatttaaggaaaaaatgcggtttagaaaaagaataa

SEQ ID NO: 38 - protein, 329 aa, Methanococcus voltae
MQVLYEADANYDKLKGKTIAVIGYGSQGRAQSLNMKESGLNVIMGLREGGASWESAKKDGHEVYSIEEAAKMADVIHILIPDEIQGNVYNSQIKPYLEEGNTLSFSHGYNIHFNYIVAPKGVNITMVAPKSPGKMVRKTYEEGFGVPGLICIEKDETGEAYDIALGMAKGIGLTRAGVIQTTFREETETDLFGEQAVLCGGVTELIKAGFETLVEAGYAPEMAYFETCHELKLIVDLIYQKGFKNMWHDVSNTAEYGGLTRRERVVTKESKEAMKEILKEIQDGRFTKEFALENQAGKPHLNSMRRLEGELLIEQVGADLRKKCGLEKE

SEQ ID NO: 39 - DNA, 1020 bp, Zymomonas mobilis
atgaaagtttattacgatagtgatgctgatcttgggctgatcaagtccaagaaaatcgctattcttggctatggtagccagggtcacgcccatgcacagaatttgcgcgattccggtgttgctgaagtagctattgcgcttcgtcctgattcggcttctgttaaaaaagcacaggatgctggtttcaaggttttgaccaatgctgaagccgcaaaatgggctgatatcctgatgatcttggcacctgatgaacatcaggctgctatctatgccgaagatttaaaagataatttgcgccctggtagtgcaattgcttttgctcatggtttgaatatccatttcggtctgatcgaaccccgcaaagatatcgatgttttcatgatcgcaccgaaaggcccaggtcacacggttcgttctgaatatgtccgtggcggtggtgtgccttgcttggtcgccgttgatcaggatgccagcggtaacgctcatgacatcgctcttgcttatgcttctggcatcggtggcggtcgttctggtgttattgaaaccactttccgtgaagaagtcgaaaccgatttgtttggtgagcaggctgttctctgcggtggtttgactgcgcttatcacggctggttttgaaactttgactgaagccggttacgctcctgaaatggcattcttcgaatgtatgcatgaaatgaagctgatcgtggatctgatctacgaagcgggtattgccaatatgcgttattcgatttctaacactgccgaatatggtgatatcgtatctggcccgcgggtcatcaatgaagaatccaaaaaggcaatgaaggctattctggacgacatccagagcggtcgttttgtcagcaaatttgttcttgataaccgcgctggtcagccggaactcaaagctgcccgtaaacgtatggctgctcacccgatcgaacaggttggtgcacgtctgcgtaaaatgatgccgtggatcgccagcaacaagctggttgataaggctcgcaactag

SEQ ID NO: 40 - protein, 339 aa, Zymomonas mobilis
MKVYYDSDADLGLIKSKKIAILGYGSQGHAHAQNLRDSGVAEVAIALRPDSASVKKAQDAGFKVLTNAEAAKWADILMILAPDEHQAAIYAEDLKDNLRPGSAIAFAHGLNIHFGLIEPRKDIDVFMIAPKGPGHTVRSEYVRGGGVPCLVAVDQDASGNAHDIALAYASGIGGGRSGVIETTFREEVETDLFGEQAVLCGGLTALITAGFETLTEAGYAPEMAFFECMHEMKLIVDLIYEAGIANMRYSISNTAEYGDIVSGPRVINEESKKAMKAILDDIQSGRFVSKFVLDNRAGQPELKAARKRMAAHPIEQVGARLRKMMPWIASNKLVDKARN

SEQ ID NO: 41 - DNA, 1020 bp, Erythrobacter sp.
atgaaagtctactacgacgccgatgccgatcttggcctcatcaaatccaagaagatcgccgtgctcggctatggctcgcagggccacgctcacgcccagaacctgcgcgacagcggtgtcgccgaagtggcaattgcccttcgcgaaggctcggccacagcaaagaaggcgcaagatgcaggcttcaaggtgctttccaataccgaggctgccaagtgggccgatatcgtgatgatcctcgcacccgatgagcatcaggcagcaatctgggaaaatgatctcgccggccacatgaagccgggcagcgcgattgcctttgcccacggcctcaacattcacttcggccttatcgaagcaccgcaggatatcgacgtcatcatgatcgcgcccaaaggtccggggcacactgtgcgtagcgaataccagcgcggcggcggcgtcccttgcctgattgctgttcatcaggacgcgagcggcagcgccaaggaaatcgccctcgcctacgcatcaggcgtcggaggcggccgctcgggcatcatcgagaccaacttccgcgaggaatgcgagaccgatctgttcggtgagcaggccgtgctttgcggcgggatcacgcacctgattcaggccggtttcgaaaccctgaccgaagccggttacgcgcccgaaatggcctatttcgagtgcctccacgaaaccaagctgatcgtcgatcttctctacgaaggcggcattgcgaacatgcgctattccatctcgaacacggcggaatatggcgacatcaccaccggcccgcgcatcatcaccgatgagacgaaggccgagatgaagcgcgtgctcgacgacatccaatcgggccgcttcgtgaagaacttcgtgctcgacaatcgcgcaggccagcccgaactcaaggcagcccgcaagcgcgccgaagctcacccgattgagaagaccggcgcagaactgcgcgcaatgatgccatggatcagtgccaacaagctggtcgacaagtcgaaaaactag

SEQ ID NO: 42 - protein, 339 aa, Erythrobacter sp.
MKVYYDADADLGLIKSKKIAVLGYGSQGHAHAQNLRDSGVAEVAIALREGSATAKKAQDAGFKVLSNTEAAKWADIVMILAPDEHQAAIWENDLAGHMKPGSAIAFAHGLNIHFGLIEAPQDIDVIMIAPKGPGHTVRSEYQRGGGVPCLIAVHQDASGSAKEIALAYASGVGGGRSGIIETNFREECETDLFGEQAVLCGGITHLIQAGFETLTEAGYAPEMAYFECLHETKLIVDLLYEGGIANMRYSISNTAEYGDITTGPRIITDETKAEMKRVLDDIQSGRFVKNFVLDNRAGQPELKAARKRAEAHPIEKTGAELRAMMPWISANKLVDKSKN

SEQ ID NO: 43 - DNA, 1020 bp, Sphingomonas wittichii
atgcgcgtctattatgatcgtgatgccgacatcggcctcatcaagaccaagaaggtggcgatcgtcggctatggcagccagggccacgcccatgcccagaacctgcaggactcgggcgtcgccgacgtcgcgatcgcgctgcgccccggctcggccaccgcgaagaaggccgaaggcgccggcttcaaggtgctgtcgaacgccgacgcggccaagtgggccgacatcgtcatgatcctggcgcccgacgagcaccaggccgcgatctacaatgacgacctgcgcgacaatctgaagccgggcgcggcgctcgccttcgcccatggcctcaacgtccatttcggcctgatcgagccgcgcgccgacatcgacgtgttcatgatcgcgccgaagggccccggccacaccgtccgttccgaatatcagcgcggcggcggcgtgccctgcctgatcgcgatcgcccaggacgccagcggcaacgcgcacgacgtcgccctgtcctacgcctcggcgatcggcggcggccgttcgggcgtgatcgagacgaccttcaaggaagagtgcgagaccgacctgttcggcgagcaggcggtgctgtgcggcggcctcagccacctgatcatggccggcttcgagacgctggtcgaggcgggctacgcccccgagatggcctatttcgaatgcctccacgaagtgaagctgatcgtcgacctgatgtatgagggcggcatcgccaacatgcgctactcgatctcgaacaccgccgaatatggcgacatccacaccggcccgcgcgtcatcacctcggagaccaaggccgagatgaagcgcgtgctcgacgacatccagaagggcaagttcgtcaagcgcttcgtcctcgacaaccgcgccggccagcccgagctgaaggcgagccgcaagctcgtcgccgagcatccgatcgagaaggtcggcgccgaactgcgcgcgatgatgccctggatcagcaagaaccagctggtcgacaaggccaagaactga

SEQ ID NO: 44 - protein, 339 aa, Sphingomonas wittichii
MRVYYDRDADIGLIKTKKVAIVGYGSQGHAHAQNLQDSGVADVAIALRPGSATAKKAEGAGFKVLSNADAAKWADIVMILAPDEHQAAIYNDDLRDNLKPGAALAFAHGLNVHFGLIEPRADIDVFMIAPKGPGHTVRSEYQRGGGVPCLIAIAQDASGNAHDVALSYASAIGGGRSGVIETTFKEECETDLFGEQAVLCGGLSHLIMAGFETLVEAGYAPEMAYFECLHEVKLIVDLMYEGGIANMRYSISNTAEYGDIHTGPRVITSETKAEMKRVLDDIQKGKFVKRFVLDNRAGQPELKASRKLVAEHPIEKVGAELRAMMPWISKNQLVDKAKN

SEQ ID NO: 45 - DNA, 1020 bp, Sphingobium japonicum
atgaaggtttattacgaccgcgacgccgacatcggcctgatcaagggcaagaaggtcgccatcctcggttacggttcgcagggtcacgcccatgcgcagaatctgcgcgacagcggcgtcgccgaagtcgccatcgcgctgcgccccggctcgcccagcgccaagaaggccgaaggcgcgggcttcaaggtgctgccgaacgcggaagccgccgcatgggccgacgtgctgatgatcctggcgcccgacgagcatcaggccgccatctacgccgccgacatccacgccaatctgcgccccggcgcggcgctggccttcgcgcacggcctcaacgtccatttcggcctgatcgagccgcgcaaggatgtcgacgtcatcatgatcgcgcccaagggtccgggccacaccgttcgcggcgaatatgtgaagggcggcggcgtgccctgcctgatcgccgttcatcaggacgcgaccggcaacgcgcatgacatcgccctgtcctacgcttcgggcgtcggcggcggccgcagcggcatcatcgaaaccaatttccgcgaggaatgcgaaaccgacctgttcggcgagcaggccgtgctgtgcggcggcgcgaccgcgctggtccaagcgggcttcgaaacgctggtcgaggcgggctacgcccccgaaatggcctatttcgaatgcctgcacgaactgaagctgatcgtcgacctgatgtatgaaggcggcatcgccaacatgcgctattcgatctcgaacaccgccgaatatggcgatatcaagaccggcccgcgcatcatcaccgaagaaacgaagaaggaaatgaagcgcgttctggccgacatccagtcgggccgcttcgtcaaggacttcgtgctcgacaaccgcgccggccagccggaattgaaggccagccgcatcgccgcccagcgccacccgatcgaggaaaccggcgccaagctgcgcgccatgatgccctggatcggcgcgaacaagctggtcgacaaggacaggaactga

SEQ ID NO: 46 - protein, 339 aa, Sphingobium japonicum
MKVYYDRDADIGLIKGKKVAILGYGSQGHAHAQNLRDSGVAEVAIALRPGSPSAKKAEGAGFKVLPNAEAAAWADVLMILAPDEHQAAIYAADIHANLRPGAALAFAHGLNVHFGLIEPRKDVDVIMIAPKGPGHTVRGEYVKGGGVPCLIAVHQDATGNAHDIALSYASGVGGGRSGIIETNFREECETDLFGEQAVLCGGATALVQAGFETLVEAGYAPEMAYFECLHELKLIVDLMYEGGIANMRYSISNTAEYGDIKTGPRIITEETKKEMKRVLADIQSGRFVKDFVLDNRAGQPELKASRIAAQRHPIEETGAKLRAMMPWIGANKLVDKDRN

SEQ ID NO: 47 - DNA, 1020 bp, Erythrobacter litoralis
gtgaaagtttattacgacgccgatgccgatcttggactgatcacggacaagaagatcgccgtgctcggctatggcagccaggggcacgcccatgcacagaatctgcgtgacagcgggatcaaagaggtagcaatcgcgctgcgcgacggctcttccagcgcgaagaaagcgcaggatgctggcttcaaggtgctcagcaattccgacgctgccgagtgggccgatatcctgatgatcctcgcccccgacgagcaccaggcggcgatctgggcggatgaccttgcgggcaacatgaagccgggcagcgccctcgccttcgcccacgggctcaacatccacttcggcctgatcgaaccacccgccgagatcgacgtcatcatgatcgcgccgaagggtcctggtcatactgtccgcagcgagtatcagcgcggcggcggcgtgccctgcctcatcgccgtccaccaggattcgagcggcaatgccaaggacatcgccctcgcctatgccagcggtgtcggcggcgggcgcagcggcattatcgagaccaacttccgcgaggaatgcgagaccgacctgttcggcgagcaggccgtgctgtgcggcgggatcacgcacctgatccaagccggcttcgagacgctgaccgaggccggatatgcaccggagatggcctatttcgagtgcctgcacgagaccaagctgatcgtcgacctgctctacgaaggcggcatcgccaatatgcgctactcgatcagcaacaccgccgagtatggcgacatcaccaccggcccgcgcatcatcaccgatgagaccaaggccgaaatgaagcgcgttctcggcgatatccagtcgggccgcttcgtgaagaacttcgtcctcgacaaccgcgccggccagcccgaactcaaggctgcccgcaagcgcgccgaagcgcatccgatcgaacagaccggtgccaagctgcgcgcaatgatgccgtggatcggcaagaacaagctggtcgacaaggacaggaactag

SEQ ID NO: 48 - protein, 339 aa, Erythrobacter litoralis
MKVYYDADADLGLITDKKIAVLGYGSQGHAHAQNLRDSGIKEVAIALRDGSSSAKKAQDAGFKVLSNSDAAEWADILMILAPDEHQAAIWADDLAGNMKPGSALAFAHGLNIHFGLIEPPAEIDVIMIAPKGPGHTVRSEYQRGGGVPCLIAVHQDSSGNAKDIALAYASGVGGGRSGIIETNFREECETDLFGEQAVLCGGITHLIQAGFETLTEAGYAPEMAYFECLHETKLIVDLLYEGGIANMRYSISNTAEYGDITTGPRIITDETKAEMKRVLGDIQSGRFVKNFVLDNRAGQPELKAARKRAEAHPIEQTGAKLRAMMPWIGKNKLVDKDRN

SEQ ID NO: 49 - DNA, 1020 bp, Sphingobium chlorophenolicum
atgaaggtttattacgaccgcgacgcagacatcggcctgatcaagggcaagaaggtcgccatcctgggttacggttcgcagggtcacgcccatgcgcagaatctgcgcgattccggcgtcgccgaagtcgccatcgcgctgcgccccggctcgccgagcgccaagaaggccgaaggcgcgggcttcaaggtgctggcgaacgccgacgccgccgcatgggccgatgtgctcatgatcctggcgcccgacgagcatcaggccgccatctacgccgacgacatccacgccaatctgcgccccggcgccgcgctcgccttcgcgcacggcctcaacgtgcatttcggcctgatcgagccgcgcaaggacgtcgacgtcatcatgatcgcgcccaagggccccggccacaccgtgcgcggcgaatatgtgaagggcggcggcgtgccctgcctgatcgccatccatcaggacgcgaccggcaacgcccatgacatcgccctgtcctacgcttcgggcgtcggcggcggccgcagcggcatcatcgaaaccaacttccgcgaggaatgcgaaaccgacctgttcggcgagcaggccgtgctgtgcggcggcgccaccgcgctggtgcaggcgggcttcgaaacgctggtcgaggctggctacgccccggaaatggcctatttcgaatgcctgcacgaactgaagctgatcgtcgacctgatgtatgaaggcggcatcgccaacatgcgctattcgatctcgaacaccgccgaatatggcgatatcaagaccggcccgcgcatcatcaccgatgaaacgaagaaggaaatgaagcgcgttctggccgacatccagtcgggccgcttcgtcaaggacttcgtgctcgacaaccgcgccggccagccggaattgaaggccagccgcatcgccgcccagcgccacccgatcgaggaaaccggcgccaagctgcgcgccatgatgccctggatcggcgcgaacaagctggtcgacaaggacaagaactga

SEQ ID NO: 50 - protein, 339 aa, Sphingobium chlorophenolicum
MKVYYDRDADIGLIKGKKVAILGYGSQGHAHAQNLRDSGVAEVAIALRPGSPSAKKAEGAGFKVLANADAAAWADVLMILAPDEHQAAIYADDIHANLRPGAALAFAHGLNVHFGLIEPRKDVDVIMIAPKGPGHTVRGEYVKGGGVPCLIAIHQDATGNAHDIALSYASGVGGGRSGIIETNFREECETDLFGEQAVLCGGATALVQAGFETLVEAGYAPEMAYFECLHELKLIVDLMYEGGIANMRYSISNTAEYGDIKTGPRIITDETKKEMKRVLADIQSGRFVKDFVLDNRAGQPELKASRIAAQRHPIEETGAKLRAMMPWIGANKLVDKDKN

SEQ ID NO: 51 - DNA, 1020 bp, Sphingomonas sp.
atgcgtgtctattatgatcgcgacgccgatctgaacctgatctccgagaagaacatcgccatcctgggttacggctcgcagggccatgcccatgcgcagaacctgcgcgattcgggcgtcaagaatgtcgcgatcgcgctgcgccccggttcggcctcggccgccaaggcggaggctgcgggcttcaaggtcctgtcgaacaaggaagcggccggctgggccgacatcctgatgatcctggcccccgacgagcatcaggccgcgatctatgacgccgacctgaagggcaatttgaagccgggcgccgcgctcgccttcgcgcacggcctgaacgtgcatttcggcctgatcgagccgcctgcggacatcgacgttatcatgatcgcgccgaagggccccggccacaccgtgcgcagcgaatatgtgcgcggcggcggcgtgccctgcctgatcgcgatccatcaggacgccagcggcaacgcgcatgacgtggcgctggcctatgcgtcgggcgtcggcggcggtcgctcgggcatcatcgagacgaacttccgcgaagagtgcgaaaccgatctgttcggcgagcaggccgtgctgtgtggcggcgcgaccgcgctggtccaggcgggcttcgagacgctggtcgaggcgggctatgcccccgaaatggcgtatttcgagtgcctccacgagctgaagctgatcgtcgacctgatgtatgagggcggcatcgccaacatgcgctactcgatctcgaacaccgccgagtacggcgacatcaagaccggcccacgcatcatcactgaagagaccaagaaggaaatgaagcgcgtgctcgccgacatccagtcgggccgcttcgtgaaggacttcgtgctcgacaaccgcgccggccagcccgaactgaaggccagccgcatcgccgccaagcgccatcagatcgaacaggtcggcagcgaactgcgcgcgatgatgccgtggatcggcgcgaacaagctggtggacaaggcgaagaactga

SEQ ID NO: 52 - protein, 339 aa, Sphingomonas sp.
MRVYYDRDADLNLISEKNIAILGYGSQGHAHAQNLRDSGVKNVAIALRPGSASAAKAEAAGFKVLSNKEAAGWADILMILAPDEHQAAIYDADLKGNLKPGAALAFAHGLNVHFGLIEPPADIDVIMIAPKGPGHTVRSEYVRGGGVPCLIAIHQDASGNAHDVALAYASGVGGGRSGIIETNFREECETDLFGEQAVLCGGATALVQAGFETLVEAGYAPEMAYFECLHELKLIVDLMYEGGIANMRYSISNTAEYGDIKTGPRIITEETKKEMKRVLADIQSGRFVKDFVLDNRAGQPELKASRIAAKRHQIEQVGSELRAMMPWIGANKLVDKAKN

SEQ ID NO: 53 - DNA, 1020 bp, Novosphingobium nitrogenifigens
atgaaggtttactacgacgccgacgccgatctcaacctgatcaccgggaagaaggtcgccatcctgggctatggcagccagggccacgcccacgcgcagaacctgcgcgattcgggcgtcaaggaagtggcgatcgcgctgcgtcccggttcggccagcgccgccaaggctgaaggcgccggtttcaaggtcatggcgaacgccgaagccgctgcctgggccgacgttctcatgattctcgcgcccgacgaacatcaggccgcgatctatgccgacgacatccacgccaacctgcgccccggcgcggcgctggccttcgcacacggcctcaacgttcacttcggcctgatcgaaccgcgcgccgacgtcgacgtgatcatgatcgcgcccaagggcccgggccacaccgttcgcggtgaatacgtgaagggcggcggggtgccctgcctcatcgccatcgcgcaggacgcgaccggcaatgcccacgacatcgcccttgcctatgcttcgggtgtcggcggcggccgttcgggtatcatcgaaaccaacttcaaggaagagtgcgaaaccgacctgttcggcgaacaagccgttctttgcggcggcctgacccacctcatccaggctggtttcgaaaccctggtcgaagccggttacgcgccggaaatggcctatttcgaatgcctccacgaagtgaagctgatcgtcgacctgatgtatgaaggcggcatcgccaacatgcgctactcgatctcgaacacggccgaatacggtgacatcaccactggtccgcgcctgatcaccgccgaaaccaaggcggaaatgaagcgcgtcctcgaagacatccaggccggtcgcttcgtcaagaacttcgtgctcgacaaccgcgctggccagcccgagctgaaggctgcccgcaaggctgccgctgcgcacccgatcgaacagaccggcgctcgcctgcgtgcgatgatgccctggatcggtgcgaaccagctggtcgacaaggccaagaactga

SEQ ID NO: 54 - protein, 339 aa, Novosphingobium nitrogenifigens
MKVYYDADADLNLITGKKVAILGYGSQGHAHAQNLRDSGVKEVAIALRPGSASAAKAEGAGFKVMANAEAAAWADVLMILAPDEHQAAIYADDIHANLRPGAALAFAHGLNVHFGLIEPRADVDVIMIAPKGPGHTVRGEYVKGGGVPCLIAIAQDATGNAHDIALAYASGVGGGRSGIIETNFKEECETDLFGEQAVLCGGLTHLIQAGFETLVEAGYAPEMAYFECLHEVKLIVDLMYEGGIANMRYSISNTAEYGDITTGPRLITAETKAEMKRVLEDIQAGRFVKNFVLDNRAGQPELKAARKAAAAHPIEQTGARLRAMMPWIGANQLVDKAKN

SEQ ID NO: 55 - DNA, 1080 bp, Bacteroides thetaiotaomicron
atggcgcaagtcatcaaaacaaaaaaacaaaaaaaaatggcacagttgaattttggcggaactgtagaaaatgtagttatccgtgatgaatttccattggaaaaagctcgtgaagtattgaaaaatgaaacaatcgctgtaatcggttatggcgtacaaggtcctggacaggctctgaaccttcgtgataacggtttcaatgtaatcgttggtcaacgccagggaaagacatatgacaaagcggtagctgacggatgggttccgggtgaaactttgttcggtattgaagaagcttgcgaaaaaggtacgatcattatgtgcctgttgtctgatgcagcggtaatgtctgtatggcctactatcaagccttacctgactgcaggaaaagctctttatttctctcatggttttgctattacatggagtgatcgcacaggtgtagttcctcctgcagatatcgacgtaatcatggttgctcctaaaggttcgggtacatccttgcgtactatgttccttgaaggtcgcggcttgaactcttcttacgctatctatcaggatgcaacaggcaacgctatggacagaacaatcgcattgggtatcggtatcggttcaggttatttgttcgaaacaactttcatccgcgaagctacttccgacctgacaggcgaacgtggttcattgatgggagctatccagggtctgttgctggcacaatacgaagtgttacgtgaaaacggtcacactccttccgaagcattcaacgaaactgtagaagagctgactcagtcattgatgccgttgtttgcaaagaacggtatggactggatgtatgctaactgctctactacagctcaacgtggtgctctcgactggatgggccccttccacgatgctatcaaaccggtagttgaaaagttgtatcacagtgtgaagactggtaacgaagcacagatttcaatcgactctaactccaaaccggattatcgtgagaaactggaagaagaactgaaagcattgcgcgaaagcgaaatgtggcagactgccgtgacagttcgtaaacttcgtccggaaaataattaa

SEQ ID NO: 56 - protein, 359 aa, Bacteroides thetaiotaomicron
MAQVIKTKKQKKMAQLNFGGTVENVVIRDEFPLEKAREVLKNETIAVIGYGVQGPGQALNLRDNGFNVIVGQRQGKTYDKAVADGWVPGETLFGIEEACEKGTIIMCLLSDAAVMSVWPTIKPYLTAGKALYFSHGFAITWSDRTGVVPPADIDVIMVAPKGSGTSLRTMFLEGRGLNSSYAIYQDATGNAMDRTIALGIGIGSGYLFETTFIREATSDLTGERGSLMGAIQGLLLAQYEVLRENGHTPSEAFNETVEELTQSLMPLFAKNGMDWMYANCSTTAQRGALDWMGPFHDAIKPVVEKLYHSVKTGNEAQISIDSNSKPDYREKLEEELKALRESEMWQTAVTVRKLRPENN

SEQ ID NO: 57 - DNA, 1215 bp, Schizosaccharomyces pombe
atgtctttccgtaattcctctagaatggccatgaaggccttgcgtactatgggtagccgtcgtttggctactcgtagcatgtctgttatggctcgcaccattgctgcccccagcatgcgttttgcgcctcgcatgaccgcccctttgatgcaaactcgcggtatgcgtgttatggactttgccggtaccaaggagaacgtttgggagcgttctgactggcctcgtgaaaagcttgttgactacttcaagaacgacactcttgccatcattggatacggatctcaaggacatggtcaaggtttgaacgctcgtgatcaaggtttgaacgttattgtcggtgtccgtaaggatggtgcttcctggaagcaagctattgaagacggttgggtccctggtaagactttgttccccgtcgaggaggccatcaagaagggttctatcatcatgaaccttttgtccgatgccgctcaaactgagacttggcccaagattgctccccttattaccaagggtaagactttgtacttctctcacggtttctccgtcatcttcaaggatcaaactaagattcacccccctaaggatgttgatgttatccttgtcgctcccaagggttctggtcgtaccgttcgtacccttttcaaggaaggtcgtggtattaactcttccttcgctgtttaccaagacgttactggtaaggctcaagaaaaggccattggtttggctgttgccgtcggttccggtttcatctaccaaaccactttcaagaaggaggttatctccgatttggttggtgagcgtggatgtctcatgggtggtatcaacggtcttttcttggctcaataccaagttttgcgtgaacgtggtcactcccctgctgaggctttcaacgagactgttgaagaggccactcaatccctttaccccttgattggcaagtacggtcttgactacatgtttgccgcttgctctaccaccgctcgtcgtggtgccattgactggactcctcgtttccttgaggctaacaagaaggtccttaatgaattgtatgacaatgttgagaacggtaacgaggctaagcgttccttggaatacaactctgctcccaactaccgtgagctttacgataaggagttggaggaaatccgcaacttggaaatctggaaggctggtgaggttgttcgttctctccgtcctgaacacaacaagcactag

SEQ ID NO: 58 - protein, 404 aa, Schizosaccharomyces pombe
MSFRNSSRMAMKALRTMGSRRLATRSMSVMARTIAAPSMRFAPRMTAPLMQTRGMRVMDFAGTKENVWERSDWPREKLVDYFKNDTLAIIGYGSQGHGQGLNARDQGLNVIVGVRKDGASWKQAIEDGWVPGKTLFPVEEAIKKGSIIMNLLSDAAQTETWPKIAPLITKGKTLYFSHGFSVIFKDQTKIHPPKDVDVILVAPKGSGRTVRTLFKEGRGINSSFAVYQDVTGKAQEKAIGLAVAVGSGFIYQTTFKKEVISDLVGERGCLMGGINGLFLAQYQVLRERGHSPAEAFNETVEEATQSLYPLIGKYGLDYMFAACSTTARRGAIDWTPRFLEANKKVLNELYDNVENGNEAKRSLEYNSAPNYRELYDKELEEIRNLEIWKAGEVVRSLRPEHNKH

SEQ ID NO: 59 - DNA, 1215 bp, Schizosaccharomyces pombe
atgtctttccgtaattcctctagaatggccatgaaggccttgcgtactatgggtagccgacgtttggctactcgtagcatgtctgttatggctcgcaccattgctgcccccagaatgcgttgggcgcctcgcatgaccgcccctttgatgcaaactcgcggtatgcgtgttatggactttgccggtaccaaggagaacgtttgggagcgctctgactggcctcgtgaaaagcttgttgactacttcaagaacgacactcttgccatcattggatccggatctcaaggacatggtcaaggtttgaacgctcgtgatcaaggtttgaacgttattgtcggtgtccgtaaggatggtgcttcctggaagcaagctattgaagacggttgggtccctggtaagactttgttccccgtcgaggaggccatcaagaagggttctatcatcatgaaccttttgtccgatgccgctcaaactgagacttggcccaagattgctccccttattaccaagggtaagactttgtacttctctcacggtttctccgtcatcttcaaggatcaaactaagattcacccccctaaggatgttgatgttatccttgtcgctcccaagggttctggtcgtaccgttcgtacccttttcaaggaaggtcgtggtattaactcttccttcgctgtttaccaagacgttactggtaaggctcaagaaaagaccattggtttggctgttgccgtcggttccggtttcatctaccaaaccactttcaagaaggaggttatctccgatttggttggtgagcgtggatgtctcatgggtggtatccccggtcttttcttggctcaataccaagttttgcgtgaacgtggtcactcccctgctgaggctttccccgagactgttgaagaggccactcaatccctttaccccttgattggcaagtacggtcttgactacatgtttgccgcttgctctaccaccgctcgtcgtggtgccattgactggactcctcgtttccttgaggctaacaagaaggtccttaatgaattgtatgacaatgttgagaacggtaacgaggctaagcgttccttggaatacaactctgctcccaactaccgtgagctttacgataaggagttggaggaaatccgcaacttggaaatctggaaggctggtgaggttggtcggtctctccggcctgaacacaacaagcactag

SEQ ID NO: 60 - protein, 404 aa, Schizosaccharomyces pombe
MSFRNSSRMAMKALRTMGSRRLATRSMSVMARTIAAPRMRWAPRMTAPLMQTRGMRVMDFAGTKENVWERSDWPREKLVDYFKNDTLAIIGSGSQGHGQGLNARDQGLNVIVGVRKDGASWKQAIEDGWVPGKTLFPVEEAIKKGSIIMNLLSDAAQTETWPKIAPLITKGKTLYFSHGFSVIFKDQTKIHPPKDVDVILVAPKGSGRTVRTLFKEGRGINSSFAVYQDVTGKAQEKTIGLAVAVGSGFIYQTTFKKEVISDLVGERGCLMGGIPGLFLAQYQVLRERGHSPAEAFPETVEEATQSLYPLIGKYGLDYMFAACSTTARRGAIDWTPRFLEANKKVLNELYDNVENGNEAKRSLEYNSAPNYRELYDKELEEIRNLEIWKAGEVGRSLRPEHNKH

SEQ ID NO: 61 - DNA, 1212 bp, Schizosaccharomyces japonicus
atgtctttccgttccgcctctaagttggccatgaaggccttccgccaaaatggtgcccgccgcgttatccctgctacccgctctatgtctgtcttggctcgcgccggcgttatgagccgctctgctgctcgtgttgcccccatggtccaaacccgtggtgtccgtactatggactttggcggtgttaaggagaccgtctgggagcgtaacgactggccccgtgagaagcttcttgactacttcaagaacgacactcttgccgttattggttacggttcccaaggtcacggacaaggtttgaacgctcgtgacaacggtttgaacgttattgttggtgttcgtgagggtggtgcctcctggaaggctgccatcgaggacggttgggtccccggcaagaacttgttccccatggaggaggccatcaagaagggtaccatcatcatggaccttctttccgatgccgctcaaaccgagacctggcccactattgctccccttctcaccaagggtaagactctctacttctctcacggtttctccgtcgtcttcaaggaccaaaccaaggttgtcccccctaaggacatcgatgtcatccttgctgcccccaagggttccggccgtaccgtccgttccttgttcaaggagggccgtggtatcaactcctccgtcgccgtcttccaaaacgtttccggcaaggctgacgagaaggctgttgctattgctgtcgccattggctccggtttcatctacaagaccaccttcgagcgcgaggtcgtttctgacttggtcggtgagcgtggttgccttatgggtggtatcaacggtctcttcttggcccaataccaaactctccgtgagcacggccacacccccgccgaggccttcaacgagactgttgaggaggccactcaatccctttaccccttgatcggtaagtacggtttggactacatgttcgccgcctgctctaccactgctcgtcgtggtgccattgactggactcaacgcttctacgacgccaacaagaaggtccttgaggacttgtacgagaacgttgccaacggtaacgaggccaagcgctccttggagtacaactctaagcccaactaccgtgagctttacgagaaggagctcgctgagatccgcgacttggagatctggagagccggtgagactgtccgttctctccgtcccgaggagaacaagcactag

SEQ ID NO: 62 - protein, 403 aa, Schizosaccharomyces japonicus
MSFRSASKLAMKAFRQNGARRVIPATRSMSVLARAGVMSRSAARVAPMVQTRGVRTMDFGGVKETVWERNDWPREKLLDYFKNDTLAVIGYGSQGHGQGLNARDNGLNVIVGVREGGASWKAAIEDGWVPGKNLFPMEEAIKKGTIIMDLLSDAAQTETWPTIAPLLTKGKTLYFSHGFSVVFKDQTKVVPPKDIDVILAAPKGSGRTVRSLFKEGRGINSSVAVFQNVSGKADEKAVAIAVAIGSGFIYKTTFEREVVSDLVGERGCLMGGINGLFLAQYQTLREHGHTPAEAFNETVEEATQSLYPLIGKYGLDYMFAACSTTARRGAIDWTQRFYDANKKVLEDLYENVANGNEAKRSLEYNSKPNYRELYEKELAEIRDLEIWRAGETVRSLRPEENKH

SEQ ID NO: 63 - DNA, 1476 bp, Salmonella enterica
atggctaactactttaatacactgaatctgcgccagcagctggcgcagctgggtaaatgccgctttatgggccgcgacgaattcgccgacggcgcgagctaccttcagggtaaaaaagtggtcatcgtcggctgtggcgctcaggggctgaaccagggcctgaacatgcgtgactccggtctggatatttcctatgccctgcgtaaagaagccattgctgagaagcgtgcctcctggcgtaaagcgaccgaaaacggcttcaaagtgggtacctacgaagagctgattccgcaggctgacctggtggttaacctgacgccggacaaacagcactccgacgtggtgcgctccgtacagccgctgatgaaagacggcgcggcgctgggctactcccacggcttcaatatcgtggaggtgggcgagcagatccgtaaagacatcaccgtggtgatggtagcgccgaagtgtccgggcaccgaagtgcgcgaagagtacaaacgtggtttcggcgtgccgacgctgatcgccgttcacccggaaaacgatccgaaaggcgaaggcatggcgattgctaaagcctgggcagcagcaactggcggtcaccgtgcgggcgtactggaatcttctttcgtggcggaagtgaaatccgacctgatgggcgagcagactatcctgtgcggtatgctgcaggctggttctctgctgtgcttcgacaagctggtggcagaaggcaccgacccggcatacgccgaaaaactgattcagttcggctgggaaaccatcaccgaagcgctgaagcagggcggcatcaccctgatgatggaccgtctgtctaacccggcgaaactgcgtgcttacgcgctgtccgaacagctgaaagagatcatggcgccgctgttccagaaacacatggatgacatcatctccggcgagttctcttccggcatgatggctgactgggctaacgacgataagaaactgctgacctggcgtgaagagaccggtaaaactgcgttcgaaaccgcgccgcagtttgaaggtaagatcggcgagcaggagtacttcgataaaggcgtgctgatgatcgcgatggtgaaagcgggcgttgagctggcgttcgaaaccatggtcgattccggcatcatcgaagaatccgcttactacgaatcactgcacgagctgccgctgatcgcgaacaccatcgcccgtaagcgtctgtacgaaatgaacgtggttatctccgataccgcagaatacggtaactatctgttctcttacgcttgcgtaccgctgctgaaaccgtttattgcggaattgcaaccgggcgatctgggtagtgctatcccggaaggcgcggtagacaacgcacagcttcgcgacgtgaacgacgcgattcgtagtcatgcgattgagcaggtaggtaagaaactgcgcggctatatgacggatatgaagcgtattgcggtagcgggttga

SEQ ID NO: 64 - protein, 491 aa, Salmonella enterica
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVVNLTPDKQHSDVVRSVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVAEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQFEGKIGEQEYFDKGVLMIAMVKAGVELAFETMVDSGIIEESAYYESLHELPLIANTIARKRLYEMNVVISDTAEYGNYLFSYACVPLLKPFIAELQPGDLGSAIPEGAVDNAQLRDVNDAIRSHAIEQVGKKLRGYMTDMKRIAVAG

SEQ ID NO: 65 - protein, 7 aa, Artificial Sequence
  Acetolactate synthase motif
SGPGXXN

SEQ ID NO: 66 - protein, 6 aa, Artificial Sequence
  Acetolactate synthase motif
GXXGXX

SEQ ID NO: 67 - protein, 15 aa, Artificial Sequence
  Acetolactate synthase motif
GXXXXGXXXXXAXXX

SEQ ID NO: 68 - protein, 5 aa, Artificial Sequence
  Acetolactate synthase motif
GDXXF

SEQ ID NO: 69 - protein, 9 aa, Artificial Sequence
  Dihydroxy acid dehydratase motif
SLXSRXXIA

SEQ ID NO: 70 - protein, 7 aa, Artificial Sequence
  Dihydroxy acid dehydratase motif
CDKXXPG

SEQ ID NO: 71 - protein, 10 aa, Artificial Sequence
  Dihydroxy acid dehydratase motif
GXCXGXXTAN

SEQ ID NO: 72 - protein, 5 aa, Artificial Sequence
  Dihydroxy acid dehydratase motif
GGSTN

SEQ ID NO: 73 - protein, 11 aa, Artificial Sequence
  Dihydroxy acid dehydratase motif
GPXGXPGMRXE

SEQ ID NO: 74 - protein, 10 aa, Artificial Sequence
  Dihydroxy acid dehydratase motif
ALXTDGRXSG

SEQ ID NO: 75 - protein, 7 aa, Artificial Sequence
  Dihydroxy acid dehydratase motif
GHXXPEA

SEQ ID NO: 76 - protein, 7 aa, Artificial Sequence
  2-keto-acid decarboxylase motif
FGXXGXX

SEQ ID NO: 77 - protein, 9 aa, Artificial Sequence
  2-keto-acid decarboxylase motif
XTXGXGXXX

SEQ ID NO: 78 - protein, 9 aa, Artificial Sequence
  2-keto-acid decarboxylase motif
NXXAGXXAE

SEQ ID NO: 79 - protein, 6 aa, Artificial Sequence
  2-keto-acid decarboxylase motif
XXXIXG

SEQ ID NO: 80 - protein, 8 aa, Artificial Sequence
  2-keto-acid decarboxylase motif
GDGXXQXT

SEQ ID NO: 81 - protein, 6 aa, Artificial Sequence
  Alcohol dehydrogenase motif
CXXDXH

SEQ ID NO: 82 - protein, 8 aa, Artificial Sequence
  Alcohol dehydrogenase motif
GHEXXGXV

SEQ ID NO: 83 - protein, 7 aa, Artificial Sequence
  Alcohol dehydrogenase motif
XXXGXXX

SEQ ID NO: 84 - protein, 7 aa, Artificial Sequence
  Alcohol dehydrogenase motif
CXXCXXC

SEQ ID NO: 85 - protein, 6 aa, Artificial Sequence
  Alcohol dehydrogenase motif
XXXXTX

SEQ ID NO: 86 - protein, 6 aa, Artificial Sequence
  Alcohol dehydrogenase motif
GXGXXG

SEQ ID NO: 87 - DNA, 1044 bp, Bacteroides thetaiotaomicron
atggcacagttgaattttggcggaactgtagaaaatgtagttatccgtgatgaatttccattggaaaaagctcgtgaagtattgaaaaatgaaacaatcgctgtaatcggttatggcgtacaaggtcctggacaggctctgaaccttcgtgataacggtttcaatgtaatcgttggtcaacgccagggaaagacatatgacaaagcggtagctgacggatgggttccgggtgaaactttgttcggtattgaagaagcttgcgaaaaaggtacgatcattatgtgcctgttgtctgatgcagcggtaatgtctgtatggcctactatcaagccttacctgactgcaggaaaagctctttatttctctcatggttttgctattacatggagtgatcgcacaggtgtagttcctcctgcagatatcgacgtaatcatggttgctcctaaaggttcgggtacatccttgcgtactatgttccttgaaggtcgcggcttgaactcttcttacgctatctatcaggatgcaacaggcaacgctatggacagaacaatcgcattgggtatcggtatcggttcaggttatttgttcgaaacaactttcatccgcgaagctacttccgacctgacaggcgaacgtggttcattgatgggagctatccagggtctgttgctggcacaatacgaagtgttacgtgaaaacggtcacactccttccgaagcattcaacgaaactgtagaagagctgactcagtcattgatgccgttgtttgcaaagaacggtatggactggatgtatgctaactgctctactacagctcaacgtggtgctctcgactggatgggccccttccacgatgctatcaaaccggtagttgaaaagttgtatcacagtgtgaagactggtaacgaagcacagatttcaatcgactctaactccaaaccggattatcgtgagaaactggaagaagaactgaaagcattgcgcgaaagcgaaatgtggcagactgccgtgacagttcgtaaacttcgtccggaaaataattaa

SEQ ID NO: 88 - protein, 347 aa, Bacteroides thetaiotaomicron
MAQLNFGGTVENVVIRDEFPLEKAREVLKNETIAVIGYGVQGPGQALNLRDNGFNVIVGQRQGKTYDKAVADGWVPGETLFGIEEACEKGTIIMCLLSDAAVMSVWPTIKPYLTAGKALYFSHGFAITWSDRTGVVPPADIDVIMVAPKGSGTSLRTMFLEGRGLNSSYAIYQDATGNAMDRTIALGIGIGSGYLFETTFIREATSDLTGERGSLMGAIQGLLLAQYEVLRENGHTPSEAFNETVEELTQSLMPLFAKNGMDWMYANCSTTAQRGALDWMGPFHDAIKPVVEKLYHSVKTGNEAQISIDSNSKPDYREKLEEELKALRESEMWQTAVTVRKLRPENN

SEQ ID NO: 89 - DNA, 1053 bp, Schizosaccharomyces pombe
atgcgtgttatggactttgccggtaccaaggagaacgtttgggagcgttctgactggcctcgtgaaaagcttgttgactacttcaagaacgacactcttgccatcattggatacggatctcaaggacatggtcaaggtttgaacgctcgtgatcaaggtttgaacgttattgtcggtgtccgtaaggatggtgcttcctggaagcaagctattgaagacggttgggtccctggtaagactttgttccccgtcgaggaggccatcaagaagggttctatcatcatgaaccttttgtccgatgccgctcaaactgagacttggcccaagattgctccccttattaccaagggtaagactttgtacttctctcacggtttctccgtcatcttcaaggatcaaactaagattcacccccctaaggatgttgatgttatccttgtcgctcccaagggttctggtcgtaccgttcgtacccttttcaaggaaggtcgtggtattaactcttccttcgctgtttaccaagacgttactggtaaggctcaagaaaaggccattggtttggctgttgccgtcggttccggtttcatctaccaaaccactttcaagaaggaggttatctccgatttggttggtgagcgtggatgtctcatgggtggtatcaacggtcttttcttggctcaataccaagttttgcgtgaacgtggtcactcccctgctgaggctttcaacgagactgttgaagaggccactcaatccctttaccccttgattggcaagtacggtcttgactacatgtttgccgcttgctctaccaccgctcgtcgtggtgccattgactggactcctcgtttccttgaggctaacaagaaggtccttaatgaattgtatgacaatgttgagaacggtaacgaggctaagcgttccttggaatacaactctgctcccaactaccgtgagctttacgataaggagttggaggaaatccgcaacttggaaatctggaaggctggtgaggttgttcgttctctccgtcctgaacacaacaagcactag

SEQ ID NO: 90 - protein, 350 aa, Schizosaccharomyces pombe
MRVMDFAGTKENVWERSDWPREKLVDYFKNDTLAIIGYGSQGHGQGLNARDQGLNVIVGVRKDGASWKQAIEDGWVPGKTLFPVEEAIKKGSIIMNLLSDAAQTETWPKIAPLITKGKTLYFSHGFSVIFKDQTKIHPPKDVDVILVAPKGSGRTVRTLFKEGRGINSSFAVYQDVTGKAQEKAIGLAVAVGSGFIYQTTFKKEVISDLVGERGCLMGGINGLFLAQYQVLRERGHSPAEAFNETVEEATQSLYPLIGKYGLDYMFAACSTTARRGAIDWTPRFLEANKKVLNELYDNVENGNEAKRSLEYNSAPNYRELYDKELEEIRNLEIWKAGEVVRSLRPEHNKH

SEQ ID NO: 91 - DNA, 20 bp, Artificial Sequence
  T7_for primer
taatacgactcactataggg

SEQ ID NO: 92 - DNA, 19 bp, Artificial Sequence
  T7_rev primer
gctagttattgctcagcgg

SEQ ID NO: 93 - DNA, 40 bp, Artificial Sequence
  LlKARI_Y26NNK_for primer
atcgccgttattggannkggttcacaaggacatgcccatg

SEQ ID NO: 94 - DNA, 40 bp, Artificial Sequence
  LlKARI_Y26NNK_rev primer
catgggcatgtccttgtgaaccmnntccaataacggcgat

SEQ ID NO: 95 - DNA, 40 bp, Artificial Sequence
  LlKARI_V48NNK_for primer
caatgttatcattggtnnkaggcacggaaaatcttttgat

SEQ ID NO: 96 - DNA, 40 bp, Artificial Sequence
  LlKARI_V48NNK_rev primer
atcaaaagattttccgtgcctmnnaccaatgataacattg

SEQ ID NO: 97 - DNA, 34 bp, Artificial Sequence
  LlKARI_R49NNK_for primer
gttatcattggtgtannkcacggaaaatcttttg

SEQ ID NO: 98 - DNA, 34 bp, Artificial Sequence
  LlKARI_R49NNK_rev primer
caaaagattttccgtgmnntacaccaatgataac

SEQ ID NO: 99 - DNA, 39 bp, Artificial Sequence
  LlKARI_G51NNK_for primer
attggtgtaaggcacnnkaaatcttttgataaagctaag

SEQ ID NO: 100 - DNA, 39 bp, Artificial Sequence
  LlKARI_G51NNK_rev primer
cttagctttatcaaaagatttmnngtgccttacaccaat

SEQ ID NO: 101 - DNA, 39 bp, Artificial Sequence
  LlKARI_K52NNK_for
ggtgtaaggcacggannktcttttgataaagctaaggaa

SEQ ID NO: 102 - DNA, 39 bp, Artificial Sequence
  LlKARI_K52NNK_rev primer
ttccttagctttatcaaaagamnntccgtgccttacacc

SEQ ID NO: 103 - DNA, 37 bp, Artificial Sequence
  LlKARI_S53NNK_for primer
gtgtaaggcacggaaaannktttgataaagctaagga

SEQ ID NO: 104 - DNA, 37 bp, Artificial Sequence
  LlKARI_S53NNK_rev primer
tccttagctttatcaaamnnttttccgtgccttacac

SEQ ID NO: 105 - DNA, 37 bp, Artificial Sequence
  LlKARI_L85NNK_for primer
tttggcaccagatgagnnkcaacaatccatatacgag

SEQ ID NO: 106 - DNA, 37 bp, Artificial Sequence
  LlKARI_L85NNK_rev primer
ctcgtatatggattgttgmnnctcatctggtgccaaa

SEQ ID NO: 107 - DNA, 39 bp, Artificial Sequence
  LlKARI_I89NNK_for primer
gagttgcaacaatccnnktacgaggaggatatcaagcct

SEQ ID NO: 108 - DNA, 39 bp, Artificial Sequence
  LlKARI_I89NNK_rev primer
aggcttgatatcctcctcgtamnnggattgttgcaactc

SEQ ID NO: 109 - DNA, 58 bp, Artificial Sequence
  Ll_recomb_1a_for primer
gggcacaatgttatcattggtsyacbacacggamwatcttttgataaagctaaggaag

SEQ ID NO: 110 - DNA, 58 bp, Artificial Sequence
  Ll_recomb_1b_for primer
gggcacaatgttatcattggtsyagtgcacggamwatcttttgataaagctaaggaag

SEQ ID NO: 111 - DNA, 58 bp, Artificial Sequence
  Ll_recomb_1c_for primer
gggcacaatgttatcattggtsyatcgcacggamwatcttttgataaagctaaggaag

SEQ ID NO: 112 - DNA, 58 bp, Artificial Sequence
  Ll_recomb_1a_rev primer
cttccttagctttatcaaaagatwktccgtgtvgtrsaccaatgataacattgtgccc

SEQ ID NO: 113 - DNA, 58 bp, Artificial Sequence
  Ll_recomb_1b_rev primer
cttccttagctttatcaaaagatwktccgtgcactrsaccaatgataacattgtgccc

SEQ ID NO: 114 - DNA, 58 bp, Artificial Sequence
  Ll_recomb_1c_rev primer
cttccttagctttatcaaaagatwktccgtgcgatrsaccaatgataacattgtgccc

SEQ ID NO: 115 - DNA, 48 bp, Artificial Sequence
  Ll_recomb_2a_for primer
ggcaccagatgagrcacaacaatccatatacgaggaggatatcaagcc

SEQ ID NO: 116 - DNA, 48 bp, Artificial Sequence
  Ll_recomb_2b_for primer
ggcaccagatgagrcacaacaatccgcatacgaggaggatatcaagcc

SEQ ID NO: 117 - DNA, 48 bp, Artificial Sequence
  Ll_recomb_2c_for primer
ggcaccagatgagttgcaacaatccatatacgaggaggatatcaagcc

SEQ ID NO: 118 - DNA, 48 bp, Artificial Sequence
  Ll_recomb_2d_for primer
ggcaccagatgagttgcaacaatccgcatacgaggaggatatcaagcc

SEQ ID NO: 119 - DNA, 48 bp, Artificial Sequence
  Ll_recomb_2a_rev primer
ggcttgatatcctcctcgtatatggattgttgtgyctcatctggtgcc

SEQ ID NO: 120 - DNA, 48 bp, Artificial Sequence
  Ll_recomb_2b_rev primer
ggcttgatatcctcctcgtatgcggattgttgtgyctcatctggtgcc

SEQ ID NO: 121 - DNA, 48 bp, Artificial Sequence
  Ll_recomb_2c_rev primer
ggcttgatatcctcctcgtatatggattgttgcaactcatctggtgcc

SEQ ID NO: 122 - DNA, 48 bp, Artificial Sequence
  Ll_recomb_2d_rev primer
ggcttgatatcctcctcgtatgcggattgttgcaactcatctggtgcc

SEQ ID NO: 123 - DNA, 30 bp, Artificial Sequence
  Ll_recomb_3KS_for primer
cacggaaaatcttttgataaagctaaggaa

SEQ ID NO: 124 - DNA, 30 bp, Artificial Sequence
  Ll_recomb_3LS_for primer
cacggactatcttttgataaagctaaggaa

SEQ ID NO: 125 - DNA, 30 bp, Artificial Sequence
  Ll_recomb_3KD_for primer
cacggaaaagattttgataaagctaaggaa

SEQ ID NO: 126 - DNA, 30 bp, Artificial Sequence
  Ll_recomb_3LD_for primer
cacggactagattttgataaagctaaggaa

SEQ ID NO: 127 - DNA, 30 bp, Artificial Sequence
  Ll_recomb_3KS_rev primer
ttccttagctttatcaaaagattttccgtg

SEQ ID NO: 128 - DNA, 30 bp, Artificial Sequence
  Ll_recomb_3LS_rev primer
ttccttagctttatcaaaagatagtccgtg

SEQ ID NO: 129 - DNA, 30 bp, Artificial Sequence
  Ll_recomb_3KD_rev primer
ttccttagctttatcaaaatcttttccgtg

SEQ ID NO: 130 - DNA, 30 bp, Artificial Sequence
  Ll_recomb_3LD_rev primer
ttccttagctttatcaaaatctagtccgtg

SEQ ID NO: 131 - DNA, 36 bp, Artificial Sequence
  Ll_K52NNkS53NNK_for primer
ggtctaccacacggannknnktttgataaagctaag

SEQ ID NO: 132 - DNA, 36 bp, Artificial Sequence
  Ll_K52NNkS53NNK_rev primer
cttagctttatcaaamnnmnntccgtgtggtagacc

SEQ ID NO: 133 - DNA, 33 bp, Artificial Sequence
  E59K_recomb_rev primer
aaaagtttcgaatccatcttycttagctttatc

SEQ ID NO: 134 - DNA, 33 bp, Artificial Sequence
  E59K_recomb_for primer
gataaagctaagraagatggattcgaaactttt

SEQ ID NO: 135 - DNA, 33 bp, Artificial Sequence
  A70V_recomb_rev primer
atctgccttagctactrcttcacctacttcaaa

SEQ ID NO: 136 - DNA, 33 bp, Artificial Sequence
  A70V_recomb_for primer
tttgaagtaggtgaagyagtagctaaggcagat

SEQ ID NO: 137 - DNA, 39 bp, Artificial Sequence
  K118E/D122G_recomb_for primer
ggatacatcraagtcccagaggrcgtggacgtgtttatg

SEQ ID NO: 138 - DNA, 39 bp, Artificial Sequence
  K118E/D122G_recomb_rev primer
cataaacacgtccacgycctctgggacttygatgtatcc

SEQ ID NO: 139 - DNA, 33 bp, Artificial Sequence
  H135L _recomb_rev primer
ggtccttctaacaaggwggcctggtgcttttgg

SEQ ID NO: 140 - DNA, 33 bp, Artificial Sequence
  H135L_recomb_for primer
ccaaaagcaccaggccwccttgttagaaggacc

SEQ ID NO: 141 - DNA, 33 bp, Artificial Sequence
  T182S_recomb_rev primer
ctcttccttgaaagtgstttcaatgatgccgac

SEQ ID NO: 142 - DNA, 33 bp, Artificial Sequence
  T182S_recomb_for primer
gtcggcatcattgaaascactttcaaggaagag

SEQ ID NO: 143 - DNA, 33 bp, Artificial Sequence
  E320K_recomb_rev primer
catagcttgtctaagttytgcccctatcttttc

SEQ ID NO: 144 - DNA, 33 bp, Artificial Sequence
  E320K _recomb_for primer
gaaaagataggggcaraacttagacaagctatg

SEQ ID NO: 145 - DNA, 32 bp, Artificial Sequence
  Sh_S78D_for primer
gcacaaaagagagccgattggcaaaaagcgac

SEQ ID NO: 146 - DNA, 32 bp, Artificial Sequence
  Sh_ S78D_rev primer
gtcgctttttgccaatcggctctcttttgtgc

SEQ ID NO: 147 - DNA, 35 bp, Artificial Sequence
  Se2_S78D_for primer
gcagaaaagagagccgattggcgtaaagcgacgga

SEQ ID NO: 148 - DNA, 35 bp, Artificial Sequence
  Se2_S78D_rev primer
tccgtcgctttacgccaatcggctctcttttctgc